(19) United States
(12) Patent Application Publication
(10) Pub. No.: US 2003/0126647 A1
(43) Pub. Date: Jul. 3, 2003

(54) Method for inducing seed development by down-regulating expression of the FIS2 gene

(76) Inventors: Pierre Bilodeau, Alberta (CA); Abdul M. Chaudhury, Bonython (AU); Elizabeth S. Dennis, Yarralumla (AU); Anna M.G. Koltunow, Coromandel Valley (AU); Ming Luo, Holt (AU); William J. Peacock, Deakin (AU)

Correspondence Address: GREENLEE WINNER AND SULLIVAN P C, 5370 MANHATTAN CIRCLE, SUITE 201, BOULDER, CO, 80303, US

(21) Appl. No.: 10/231,778

(22) Filed: Aug. 28, 2002

Related U.S. Application Data
(63) Continuation of application No. 09/398,237, filed on Sep. 20, 1999, now abandoned.
(60) Provisional application No. 60/101,184, filed on Sep. 21, 1998.

(30) Foreign Application Priority Data
Sep. 22, 1998 (AU) PP6061
Sep. 22, 1998 (AU) PP6062
Sep. 22, 1998 (AU) PP6063
Jul. 1, 1999 (AU) PQ1345
Jul. 1, 1999 (AU) PQ1346

(51) Int. Cl. A01H 5/00; A01N 25/00

(52) U.S. Cl. 800/287; 504/116.1

(57) ABSTRACT

The present invention provides a method of inducing seed development in plants, preferably in the absence of sexual fertilisation, said method comprising inhibiting or preventing the expression of one or more regulatory polypeptides that otherwise prevent asexual seed development in plants. The invention further provides novel genetic sequences. The invention further provides transformed plants having a wide range of novel phenotypes including, but not limited to, the ability to reproroduce asexually, develop seed in the absence of fertilisation, and the ability to produce parthenocarpic fruit or seedless fruit or fruits with soft seed traces such that the fruit are marketable as less seedy than wild-type fruit or seedless. The isolated nucleic acid molecules are further useful in the detectrion of proteins and genetic sequences which interact with the polypeptides encoded by said nucleic acid molecules in the regulation of seed development in plants.

### CROSS REFERENCE TO RELATED APPLICATIONS

This application is a continuation of U.S. patent application Ser. No. 09/398,237, filed Sep. 20, 1999, which application claims benefit of U.S. Provisional Application No. 60/101,184 filed Sep. 21, 1998; Australian Application No. PP6061 filed Sep. 22, 1998; Australian Application PP6062 filed Sep. 22, 1998; Australian Application PP6063 filed Sep. 22, 1998; Australian Application PQ1345 filed Jul. 1, 1999; and Australian Application PQ1346 filed Jul. 1, 1999.

### FIELD OF THE INVENTION

The present invention relates generally to a method of inducing autonomous (i.e. fertilisation independent) seed development in plants, including but not limited to the induction of autonomous endosperm development and/or partial autonomous embryo development. The invention further provides genes which are capable of regulating seed development in plants and pertains to their use in preventing fertilization-dependant seed production or reducing the frequency thereof. More particularly, the present invention provides isolated nucleic acid molecules comprising nucleotide sequences which encode or are complementary to nucleotide sequences which encode regulatory polypeptides involved in the progressive development of an ovule into a seed in plants. The isolated nucleic acid molecules of the invention are useful for the production of plants having a wide range of novel phenotypes including, but not limited to, the ability to reproduce asexually, develop seed in the absence of fertilization, and the ability to produce parthenocarpic fruit or seedless fruit or fruits with soft seed traces such that the fruit are marketable as less seedy than wild-type fruit or seedless. The isolated nucleic acid molecules are further useful in the detection of proteins and genetic sequences which interact with the polypeptides encoded by said nucleic acid molecules in the regulation of seed development in plants, thereby producing a novel range of products for the genetic modification of seed development.

### BACKGROUND OF THE INVENTION

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any two or more of said steps or features.

Throughout this specification, unless the context requires otherwise the word “comprise”, and variations such as “comprises” and “comprising”, will be understood to imply the inclusion of a stated integer or step or group of integers or steps but not the exclusion of any other integer or step or group of integers or steps.

Bibliographic details of the publications referred to by author in this specification are collected at the end of the description.

This specification contains nucleotide and amino acid sequence information prepared using the programme PatentIn Version 2.0, presented herein after the bibliography. The length, type of sequence (DNA, protein (PRT), etc) and source organism for each nucleotide or amino acid sequence are indicated in the Sequence Listing. Nucleotide and amino acid sequences referred to in the specification are defined by the sequence identifier (SEQ ID NO:1, for example).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

The designation of amino acid residues referred to herein are also those recommended by the IUPAC-IUB Biochemical Nomenclature Commission, as indicated in Table 1. For those sequences comprising the variable residue Xaa (i.e. X), it will be known to those skilled in the art that two or more consecutive Xaa residues in an amino acid sequence may be identical or non-identical residues, and the present invention is not limited by any particular configuration of such sequences unless specifically stated otherwise in the specification. The amino acid designation B (Asx) is also known by those skilled in the art to indicate an occurrence of Aspartate or Asparagine at a particular position in an amino acid sequence. The amino acid designation Z (Glx) is also known by those skilled in the art to indicate an occurrence of Glutamate or Glutamine at a particular position in an amino acid sequence.

As used herein, the term “derived from” shall be taken to indicate that a particular integer or group of integers has originated from the species specified, but has not necessarily been obtained directly from the specified source.

In plants which reproduce by sexual means, the endosperm and embryo of the developing seed are normally formed from the megagametophyte (i.e. the embryo sac) which is contained within the central region of the ovules, whilst the integument(s) and other surrounding structures which enclose the megagametophyte differentiate into a seed coat. The development of the embryo sac in flowering plants can be divided into two stages, megasporogenesis and megagametogenesis. During megasporogenesis the female archesporial cells undergo meiosis and four megaspore cells are formed. The polygonum-type of embryo sac formation is the most common type observed in flowering plants occurring, for example in Arabidopsis thaliana (Mansfield et al., 1991). Polygonum-type embryo sacs form from the megaspore situated in the chalazal end of the ovule, after the three non-functional megaspores in the micropylar end degenerate. The remaining functional chalazal megaspore undergoes three successive mitotic divisions to produce the female gametophyte containing eight-nuclei.

The embryo sac develops sexual competence within the gynoecium, following nuclear migration and cellularization events. The polygonum-type embryo sac has one egg cell, two synergids, three antipodal cells and a central cell containing two nuclei. The egg cell is located at the micropylar end of the embryo sac and, following fertilization, the egg nucleus ultimately fuses with one of the male sperm nuclei to produce a zygote, the progenitor of the embryo. The egg is adjacent to two synergids which may play an important role in fertilisation by aiding in pollen tube attraction and guidance and facilitating the incorporation of the sperm nuclei into the egg and central cells.

The polar nuclei are fertilised by the other sperm nucleus, generating the triploid primary endosperm nucleus and completing the double fertilisation event characteristic of angiosperms. The mature endosperm nucleus undergoes several rounds of division without cytokinesis to generate a large number of free nuclei organised at the periphery of the central cell. Cytokinesis then ensues, progressing centripetally, until the endosperm becomes entirely cellular.

The fate of the endosperm can vary between plant species. In Arabidopsis thaliana, the endosperm is utilised during embryo development, whilst in cereals the endosperm persists.

The function of three antipodal cells located at the chalazal end of the embryo sac is not known, however they are thought to be involved in the import of nutrients to the embryo sac. In some plants, for example Arabidopsis thaliana, the antipodal cells degenerate prior to fertilisation, whilst in other plants, such as cereal crop plants, they can proliferate.

A summary of embryogenesis in Arabidopsis thaliana is presented in FIG. 1.

Little is known of the mechanism or biochemistry of ovule development or the mechanism or biochemistry of the subsequent development of the ovule into a seed. Specific regulatory mechanisms controlling such processes remain to be elucidated.

Many higher plants are capable of forming seed in the absence of fertilisation, a process known as apomixis (Asker and Jerling, 1992). Studies of fertilization-independent seed production indicates that, in such plants embryos may form inside embryo sacs derived from cells that have not undergone meiosis (i.e. apospory or diplospory) or the embryos may form directly from other maternal ovule cells. For example, in orchids, citrus and mango plants, adventitious embryos arise from the cells of the nucellus or inner integuments.

In plants such as Poa spp. and Pennisetum spp., aposporous embryo sacs may arise via mitosis from cells that differentiate from the nucellus following megaspore mother cell differentiation, wherein the aposporous embryo sac may develop more rapidly than the sexual embryo sac present in the same ovule, possibly because they are not delayed by meiosis (Koltunow, 1993). In many such cases, the development of the sexual embryo sac is often terminated (Asker and Jerling, 1992). In plants that undergo aposporous embryo sac formation, endosperm development usually, but not always, requires pseudogamy (i.e. pollination and fusion of the sperm cell with only the unreduced polar cell or equivalent), however autonomous endosperm development following aposporous embryo sac formation does occur in Hieracium spp (Asker and Jerling, 1992).

Furthermore, in diplosporous plants, meiosis may be inhibited or aberrant or aborted at an early stage during megasporogenesis (i.e. at the time the spores are formed). In Antennaria spp., the megaspore mother cell is prevented from entering meiosis or undergoes an aberrant meiosis which resembles mitosis, such that the embryo sac produced has the same number of cells as a sexual embryo sac for that species. On the other hand, in Taraxacum spp., meiosis is aborted at an early stage and mitosis-like divisions give rise to dyads, in the absence or presence of recombination. Diplospory has also been observed in Ixeris spp and in the cruciferous plant Arabis holboellii (Asker and Jerling, 1992; Bocher, 1951; Roy and Reiseberg, 1989).

Genetic control of seed development and in particular, fertilisation-independent seed development, may involve only a few genes. Adventitious embryony in citrus appears to be controlled by a single dominant locus (Parlevliet and Cameron 1959; Iwamasa et al., 1967; Asker and Jerling, 1992). Recent reports on genetic control of apospory in Pennisetum species indicate that apospory may be controlled by a single dominant gene locus (Ozias-Akins et al., 1993; 1998). Work in Panicum and Ranunculus also indicate similar control (Reviewed by Koltunow, 1993). The trait of apospory observed in Pennisetum squamulatum has been introduced to a sexual species pearl millet and the resulting apomictic line has been shown to contain a single supernumerary chromosome containing the apomictic gene from P. squamulatum. The transferred chromosome can be detected by RFLPs and molecular markers linked to apospory have recently been identified on the transferred chromosome (Ozias-Akins et al., 1993; 1998).

There have not been many reports on studies of the genetic control of diplospory, however a recent study of diplospory in Taraxacum suggests that the control of female meiosis or apomixis may reside on a single chromosome and probably at a single locus (Reviewed by Koltunow, 1993) however, the gene(s) controlling diplosporous apomixis remain to be elucidated in this species.

Regulating seed development in plants has enormous economic utility in the horticulture and agriculture industries, for example, producing soft-seeded fruit (i.e. fruit that lack an embryo and/or are shrivelled or shrunken or degenerate during development) or fruit having no seed, which fruit are more appealing to consumers, in particular with regard to edible fruits such as stone fruits, citrus fruits, grapes and melon varieties, amongst others. Additionally, plants that are capable of autonomous seed formation in the absence of fertilisation are highly desirable products. Because plants which undergo autonomous seed formation do not require fertilisation to reproduce, such plants may express desirable characteristics stably between generations.

### SUMMARY OF THE INVENTION

In work leading up to the present invention, the inventors sought to elucidate the regulatory mechanisms involved in seed and fruit development in higher plants. The inventors developed a visual screen to facilitate the identification of genes which are capable of being used to regulate the development of the ovule into seed and may be used to produce fruit having soft seed, especially in the absence of fertilization.

In particular, the inventors have chemically-mutagenised a male-sterile, but fully female-fertile plant line which is incapable of forming seed in the absence of a pollen donor, to produce plants which are both capable of forming seed in the absence of a pollen donor and capable of producing soft-seeded fruit or seedless fruit in the absence of a pollen donor. By characterising a transposon-tagged mutant which belongs to the same complementation group as the chemically-induced mutant, the inventors were able to isolate genomic DNA from the tagged mutant in the region surrounding the transposon and to demonstrate that the homologous genomic DNA derived from a wild-type plant is able to complement the mutation in genetically-transformed mutant plants. The mutated gene which has been complemented using this approach has been designated as the FIS2 gene.

The inventors have identified two additional genes, designated FIS1 and FIS3, which are also capable of regulating autonomous endosperm development and/or autonomous embryogenesis and/or autonomous seed development in plants and in particular, in Arabidopsis thaliana.

In summary, the FIS family of genes described herein have been shown by the present inventors to be at least partial negative regulators of autonomous endosperm development and/or autonomous embryogenesis.

Accordingly, one aspect of the present invention provides a method of inducing autonomous endosperm development in a plant, said method at least comprising the step of inhibiting, interrupting or otherwise reducing the expression of a negative regulator of seed formation in one or more female reproductive cells, tissues or organs of said plant or a progenitor cell, tissue or organ thereof. According to this embodiment of the invention, the reduced expression of the negative regulator is achieved by the introduction of a transgene which comprises a FIS genetic sequence in the sense or antisense orientation as described herein.

Preferably, the inventive method provides in part or whole for autonomous embryogenesis and more preferably, for autonomous seed development in plants.

In a particularly preferred embodiment, the negative regulator of seed formation is a FIS polypeptide which comprises an amino acid sequence which is at least about 50% identical to any one of SEQ ID NO:1 or SEQ ID NO:2 or SEQ ID NO:3, or alternatively or in addition which is capable of being encoded by a nucleotide sequence which is at least about 50% identical to the nucleotide sequence set forth in any one of SEQ ID NO:4, SEQ ID NO:5, SEQ ID NO:6, SEQ ID NO:7, SEQ ID NO:8 or SEQ ID NO:9, or a sequence complementary thereto.

A second aspect of the invention provides isolated nucleic acid molecules which are used to inhibit, prevent or interrupt the expression of a FIS polypeptide in a plant according to the inventive method, including those genomic equivalents of the Arabidopsis thaliana FIS polypeptides exemplified herein.

A third aspect of the invention provides a transgenic plant or a plant cell, tissue, organ produced according to the method described herein, including the seed produced by said plant and progeny plants derived therefrom which are capable of forming soft-seed in the absence of fertilisation or alternatively, which are capable of forming fully-fertile seed in the absence of fertilisation.

A further aspect of the invention provides an isolated nucleic acid molecule comprising a nucleotide sequence which encodes or is complementary to a nucleotide sequence which encodes a FIS polypeptide, protein or enzyme which is capable of regulating seed development in plants. Preferably, the subject nucleic acid molecule is involved in regulating the development of the ovule into seed in the absence of fertilization, such as by acting as a repressor of autonomous embryogenesis and/or a partial repressor of autonomous endosperm development.

In one embodiment, the isolated nucleic acid molecule of the invention encodes FIS1, a member of the E(z) class of proteins which also comprises novel amino acid sequence motifs not normally associated with this class of protein, in particular a TNFR/NGFR protein domain, an R-G-D tripeptide domain and a novel domain designated the WCA motif. The FIS1 polypeptide preferably comprises an amino acid sequence which is at least about 50% identical to the amino acid sequence set forth in SEQ ID NO:1.

In another embodiment, the isolated nucleic acid molecule of the invention encodes FIS2, a zinc-finger or zinc-finger-like protein. The invention clearly extends to isolated nucleic acid molecules which encode zinc-finger or zinc-finger-like proteins which comprises an amino acid sequence which is at least about 50% identical to the amino acid sequence set forth in SEQ ID NO:2.

In yet another embodiment, the isolated nucleic acid molecule of the invention encodes FIS3 and is capable of hybridizing under at least low stringency hybridization conditions to that region of chromosome 3 of Arabidopsis thaliana which maps between the markers m317 and DWF1 as set forth in FIG. 9B, or which is at least about 50% identical to the amino acid sequence set forth in SEQ ID NO:3.

In an alternative embodiment, the isolated nucleic acid molecule of the invention comprises a nucleotide sequence which is at least about 50% identical to the nucleotide sequences set forth in any one of SEQ ID NO:4, SEQ ID NO:5, SEQ ID NO:6, SEQ ID NO:7, SEQ ID NO:8, or SEQ ID NO:9, or a complementary nucleotide sequence thereto.

In a further alternative embodiment, the isolated nucleic acid molecule of the invention comprises a nucleotide sequence which is capable of hybridizing under at least low stringency hybridization conditions to the nucleotide sequences set forth in any one of SEQ ID NO:4, SEQ ID NO:5, SEQ ID NO:6, SEQ ID NO:7, SEQ ID NO:8, or SEQ ID NO:9, or a complementary nucleotide sequence thereto.

In a particularly preferred embodiment, the isolated nucleic acid molecule of the invention comprises the nucleotide sequence set forth in any one of SEQ ID NO:4, SEQ ID NO:5, SEQ ID NO:6, SEQ ID NO:7, SEQ ID NO:8, or SEQ ID NO:9, or a complementary nucleotide sequence thereto or a homologue, analogue or derivative of said nucleotide sequences.

A further aspect of the invention provides a cell which has been transformed or transfected with the subject nucleic acid molecule or a dominant-negative sense molecule or an antisense molecule or a ribozyme molecule or a gene-targeting molecule or a co-suppression molecule which is derived from a nucleic acid molecule comprising a FIS gene, preferably in an expressible form. The present invention clearly extends to transformed tissues, organs and whole organisms comprising the subject nucleic acid molecule or a dominant-negative sense molecule or an antisense molecule or a ribozyme molecule or a gene-targeting molecule or a co-suppression molecule which is derived from said nucleic acid molecule.

In a particularly preferred embodiment, the invention provides a plant cell, tissue, organ or whole plant which comprises the nucleic acid molecule described herein or a dominant-negative sense molecule or an antisense molecule or a ribozyme molecule or a gene-targeting molecule or a co-suppression molecule which is derived from said nucleic acid molecule. The invention extends to the progeny of such a plant, the only requirement being that said progeny also contain said nucleic acid molecule, dominant-negative sense molecule, antisense molecule, ribozyme molecule, gene-targeting molecule or a co-suppression molecule.

A still further aspect of the invention provides an isolated promoter sequence which is capable of conferring expression at least in one or more female reproductive cells, tissues or organs of said plant or a progenitor cell, tissue or organ thereof.

A still further aspect of the present invention provides an isolated or recombinant FIS polypeptide or a homologue, analogue, derivative or epitope thereof.

The recombinant FIS polypeptides or derivatives thereof comprising FIS protein domains which are involved in forming protein:protein interactions are particularly useful in the isolation of further peptides and polypeptides which are normally regulated by said FIS polypeptides. By appropriate strategies described herein, the nucleic acid molecules encoding said peptides and polypeptides may also be isolated and expressed in the cells under the control of suitable promoter sequences, such as a FIS gene promoter, to induce autonomous endosperm development and/or autonomous embryogenesis and/or autonomous or pseudogamous seed development in plants.

A further aspect of the invention extends to a monoclonal or polyclonal antibody molecule which is capable of binding to a FIS polypeptide or an epitope thereof.

### BRIEF DESCRIPTION OF THE DRAWINGS

FIG. 1 is a schematic representation showing female gametophyte, fertilisation and embryogenesis of Arabidopsis thaliana embryogenesis. (a) The ovule contains the female gametophyte composed of an egg, a 2n central cell, two synergids next to the egg, and three antipodal cells in the chalazal end. (b) Pollen tube enters the ovule through the micropyle and delivers two sperm cells that fuse with the egg and the central cell. (c) Following fertilisation, a zygote and a primary endosperm cell are produced. (d) During embryogenesis, embryo and endosperm development occurs. (e) At the end of embryogenesis a mature embryo is formed.

FIG. 2 is a schematic representation of a genetic screen used to detect autonomous endosperm mutants in Arabidopsis thaliana, showing three different types of readily distinguishable flower morphologies. Morphology type 1 is the pistillata homozygous type in which the siliques are short and there are no stamens or pollen. Morphology type 2 indicates self-fertile plants with stamens and siliques that are longer than Type 1. Morphology type 3 is the putative fis mutant. In this type, although the siliques are long, there are no petals or stamens, indicating that pistillata has not reverted (from Peacock et al., 1995).

FIG. 3 is a copy of a photographic representation showing wild-type and fis seed development. Seed development of wild-type Arabidopsis thaliana and fis mutants are compared at developmental phases (Bowman and Koornneef, 1994). Phase 1 shows ovules connected to the ovary wall by the funiculus; in the subsequent phases, only the developing seed is shown. The relative size of the ovule compared with the developing seed is shown by the Inset. The lengths of siliques at the different phases are: phase 1:0.29±0.04 mm (0 HAF); phase 2:0.60±0.08 mm (36 HAF); phase 3:1.00±0.07 mm (72 HAF); and phase 4 1.26±0.07 mm (120 HAF). a, b, and c represent different developmental types seen in the fis mutants. X, Y, and Z represent postulated genes other than FIS1, FIS2, and FIS3.

FIG. 4 is a photographic representation of cryoscanning electron micrographs of ovules and seeds of fis mutants and fertilized wild-type plants. Developing ovules [nucellar column (n) protruding from the inner integument (ii) and the outer integument (oi) as shown in B] of (A) wild-type, (B) fis1/fis1 homozygotes, (C) fis2/fis2 homozygote, and (G) FIS3/fis3 heterozygote. (D) Sexually fertilized seeds (s) of pi/pi FIS/FIS plants 7 days after fertilization. Unfertilized ovules shrivel (arrow). Seeds developing without fertilization(s) of (E) fis1/fis1 homozygote, (F) fis2/fis2 homozygotes, and (H) FIS3/fis3 heterozygote. Collumella (c) on the surface of (I) sexually fertilized seeds of wild type and (J) autonomously-developing fis2/fis2 homozygous seeds. (Bar: 20 μm for A-C, G, I, and J; 100 pm for D-F; and 200 Hm for H) (from Chaudhury et al., 1997).

FIG. 5 is a photographic representation showing various stages of embryo development in wild-type plants and fis mutant plants, as follows. Panel 1, 7-day old wild type embryo; panel 2, 7-day old fis1 mutant embryo (Ler background) arrested at the heart stage; panel 3, 7-day old fis2 mutant embryo (Ler background) arrested at the heart stage; panel 4, 7-day old fis3 mutant embryo (Ler background) arrested at the heart stage; panel 5, 7-day old fis2/fis2 homozygous mutant embryo (Col background) arrested at the heart stage; panel 6, fis2/fis2 homozygous mutant embryo (Col background) arrested at the torpedo stage; panel 7, 7-day old fis1/fis2-2 double homozygous mutant embryo arrested at the heart stage; and panel 8, well-developed embryo of fis1/fis2-2 double homozygous mutant.

FIG. 6 is a graphical representation showing the localization of the fis1 allele and the mea allele on chromosome 1 of Arabidopsis thaliana. The BAC clones I4O10 and I4J10 were isolated using the mea probe. The position of the BACs and marker genes is based on the information from the AbtD.

FIG. 7 is a graphical representation of the position of fis2 locus on chromosome 2. The relative position of the fis2 locus and RFLP markers YUP11D2R end, 11A7L end, and BAC26D2 fragment 5BC was established by examining the segregation of RFLPs in plants with recombination breakpoints in either the er-fis2 or the fis2-as interval. YUP9D3, and 11D2 were originally identified based on their location shown in the WEB site describing the Arabidopsis thaliana-mapped YACs. 11A7L end showing tight linkage with fis2 was used to isolate cosmid pOCA18H1 (in vector pOCA18). The length of YAC, BAC, and cosmid clones are shown in parentheses.

FIG. 8 is a graphical representation showing the localisation of the fis3 locus on chromosome 3, between the morphological markers hy and gl. The position of the SSLP marker nga162 and the RFLP marker ve039 are also indicated. The position of the transposable Ds element in a transposon-tagged fis3 mutant line is also indicated (DT51). Numbers in brackets refer to recombination distance (cM).

FIG. 9A is a graphical representation showing the localisation of morphological markers, cosmid clones, BAC clones, YAC clones and RFLP markers on chromosome 3 of Arabidopsis thaliana.

FIG. 9B is a graphical representation showing the localisation of morphological markers, cosmid clones, BAC clones, YAC clones and RFLP markers around the RFLP marker ve039 fis3 locus on chromosome 3 of Arabidopsis thaliana.

FIG. 10A is a graphical representation of the F1 plant P19 resulting from the cross DSG X Ac. Two sectors (branches) of this plant show fis-like phenotype, as indicated by the black circles (●), whilst the normal phenotype is indicated by the white circles (∘).

FIG. 10B is a photographic representation of a Southern blot of BamHI digested genomic DNA from the transposon-tagged plant P19 and a wild type plant. The probe used corresponds to a fragment of approximately 10 kb in length (3BB) from cosmid cos18H1 which contains fragment E2 (FIG. 11).

FIG. 11 is a schematic representation of the physical map of the cosmid pOCA18H1. The genetic loci indicated are; LB, left border repeat; NOS-NPT-OCS, a chimeric gene which is expressed in plant cells and confers resistance to kanamycin; p1AN7, contains a ColE1 plasmid origin of replication and a bacterial supF tRNA gene; COS, the cos region from phage lambda; RB, right border repeat; TET, a bacterial tetracycline resistance gene. The direction of transcription for the NOS-NPT-OCS gene is indicated by the arrow. The restriction sites indicated are: B, BamHI; C, ClaI; E, EcoRI; H, EcoRV, V; HindIII; K, KpnI; P, PstI; and S, SalI. The A. thaliana genomic DNA partially digested with TaqI was ligated in the ClaI digested pOCA18. The corresponding site of insertion of the DSG transposon in DNA obtained from the fis2-2 tagged mutant is indicated by the open triangle.

FIG. 12 is a schematic representation of a silique from fis2/FlS2 heterozygote and a silique from the cross of fis2/fis2 homozygote with transgenic A. thaliana ecotype C24 containing the T-DNA from cosmid pOCA18H1. Black circles (●) correspond to good fertile seeds and open circles (∘) correspond to sterile seeds.

FIG. 13A is a schematic representation of the single base pair changes occurring in the fis2 gene of mutant fis2-1 plants. The amino acid sequence (SEQ ID NO:211) is shown below the nucleotide sequence (SEQ ID NO: SEQ ID NO:210). Numbers on the left hand side correspond to the nucleotide sequence and numbers on the right hand side correspond to the amino acid sequence. The localization of the fis2-1 mutation (deletion of T) is shown with the resulting frame-shift. The stop codon is indicated with an asterisk (*). Lower case letters show the intron sequence.

FIG. 13B is a schematic representation of the single base pair changes occurring in the fis2 gene of mutant fis2-3 plants. The amino acid sequence (SEQ ID NO:212) is shown below the nucleotide sequence of the wild-type gene (SEQ ID NO:213). Numbers on the left hand side correspond to the nucleotide sequence and numbers on the right hand side correspond to the amino acid sequence. The nucleotide sequence around the fis2-3 mutation (G to A) at the junction of intron 5 and exon 6 is also shown.

FIG. 14 is a graphical representation of the FIS2 amino acid sequence (SEQ ID NO:2), showing the locations of the acidic regions (single underlined); the putative nuclear localization signal (NLS; double underlined) identified by functional expression studies; and the C2H2 zinc finger motif (triple underlined) including conserved cysteine and histidine residues.

FIG. 15 is a graphical representation of a bi-dimensional plot of a C-terminal region of the FIS2 predicted protein sequence showing the tandem repeats between residue 120 and 520 thereof. The dot matrix was obtained using the software Antherprot V3.2 with a window size of 19 amino acids and a identity threshold of 10. The principle of the method is described in (Staden, 1982).

FIG. 16 is a photographic representation of a Southern blot showing A. thaliana FIS2 genome organisation. Genomic DNA was digested with either BamHI, BglII, or ClaI prior to electrophoresis. The DNA was transferred onto nylon membranes and hybridized with the Fis2 cDNA insert.

FIG. 17 is a photographic representation of the expression pattern of the Fis2 transcript in root, shoot, leaf, bolt, flower and silique of wild type Arabidopsis as detected by RT-PCR analysis.

FIG. 18 is a representation showing the FIS1 nucleotide sequence (SEQ ID NO:4) and deduced amino acid sequence of thewild-type MEDEA/FIS1 polypeptide (SEQ ID NO:1). The acidic region is underlined. The C5 domain is in boldface. The cysteines of the CXC domain are are in boldface and underlined. Basic residues of a putative bi-partite nuclear localization signal are indicated by asterisks under the amino acid residues. The 115-amino acid SET domain is boxed. The position of nucleotide changes in the fis1 mutant allele and the point of insertion of the transposon in the medea mutant are indicated by the arrows.

FIG. 19 is a schematic representation showing three polycomb group polypeptides from Arabidopsis thaliana (FIS1, EZA1 and CURLY LEAF), the Drosophila melanogaster Enhancer of zeste (E[z]) polypeptide and the Caenorhabditis elegans Maternal-Effect Sterile-2 (MES-2) polypeptide. The SET domain is shown as a shaded box. The CXC domain is shown as a hatched box. Positions of the acidic domain (A), putative nuclear localization signal (N) and C5 domain are indicated. The arrows on the FIS1 protein indicate the positions of mutations in the corresponding gene which produce the fis1 mutant phenotype (black arrow) and the mea mutant phenotype (open arrow). Numbers on the right refer to the protein length in amino acid residue.

FIG. 20 is a schematic representation showing the amino acid sequence alignment of various Enhancer of zeste E(z)-like proteins around the C5 cysteine-rich domain (i.e. FIS1, SEQ ID NO: 214; EZA1, SEQ ID NO: 215; CLF, SEQ ID NO: 216; MES-2, SEQ ID NO: 217; E(z), SEQ ID NO: 218; EZH2, SEQ ID NO: 219; and Ezh1, SEQ ID NO: 220). The asterisks indicate the positions of the five conserved cysteine residues. The numbers on the right refer to amino acid positions in each complete amino acid sequence.

FIGS. 21A-21E provide a schematic representation showing the amino acid sequence alignment of FIS1 (SEQ ID NO: 1) to various Enhancer of zeste E(z)-like proteins, in particular, EZA1, SEQ ID NO: 221; CLF, SEQ ID NO: 222; MES-2, SEQ ID NO: 223; E(z), SEQ ID NO: 224; EZH2, SEQ ID NO: 225; and Ezh1, SEQ ID NO: 226. Darker shading represents highly conserved regions. The numbers on the right refer to amino acid positions in each complete amino acid sequence.

FIGS. 21A-21E provide a schematic representation showing the amino acid sequence alignment of the TNFR/NGFR domains of various Enhancer of zeste E(z)-like proteins. The first 2 TNFR/NGFR domain sequences (tnfr-r1, SEQ ID NO: 227; and tnfr-r2, SEQ ID NO: 228) are both found in the human TNFR type1 protein (Genbank P19348). The remaining 5 sequences are derived from E(z)-like proteins of Arabidopsis thaliana (FIS1, EZA1 and CURLY LEAF), Drosophila melanogaster[E(z)] and Caenorhabditis elegans (MES-2) and are set forth in amino acid sequences SEQ ID NO:229 to SEQ ID NO:234, respectively. The six conserved cysteine residues are indicated by asterisks. The numbers on the right refer to amino acid positions in each complete amino acid sequence.

FIG. 23 is a schematic representation showing the amino acid sequence alignment of the WCA domains of various Enhancer of zeste E(z)-like proteins. The sequences are derived from Arabidopsis thaliana (FIS1, EZA1 and CURLY LEAF), Drosophila melanogaster[E(z)], human (EZH2) and murine (Ezh1) E(z)-like proteins and are set forth in amino acid sequences SEQ ID NO:235 to SEQ ID NO:239, respectively. The alignment was obtained using the computer program Clustlaw and was viewed with the computer program Genedoc. The numbers on the right refer to amino acid positions in each complete amino acid sequence.

FIG. 24 is a schematic representation of the FIS1/GUS and FIS2/GUS fusion constructs, showing the positions of the FIS1 and FIS2 promoter regions (open boxes), predicted translation start site (ATG), exons (black boxed regions), and introns (thin lines). There is a further translation start site in the FIS2 gene which the inventors have foundmay be used to produce a FIS2 polypeptide, located at nucleotide positions 364 to 366 of SEQ ID NO: SEQ ID NO:6. The location of the C2H2 zinc finger motif in the FIS2 polypeptide is indicated. Numbers to the left of the schematic indicate the length of the region derived from the FIS1 and FIS2 genes, respectively that has been fused to the GUS open reading frame in these fusion constructs.

FIG. 25 is a copy of a photographic representation showing the expression of the FIS1/GUS fusion constructs depicted in FIG. 24, in the central nucleus (Panel 1); two endosperm nuclei (Panel 2); three endosperm nuclei (Panel 3); six endosperm nuclei (Panel 4); 32 endosperm nuclei (Panel 5); and endosperm cyst (Panel 6).

FIG. 26 is a copy of a photographic representation showing the expression of the FIS2/GUS fusion constructs depicted in FIG. 24, in the unfused nuclei of the central cell (Panel 1); fused nucleus of the central cell (Panel 2); two free endosperm nuclei (Panel 3); four free endosperm nuclei (Panel 4); eight free endosperm nuclei (Panel 5); 15 free endosprem nuclei (Panel 6); 30 free endosperm nuclei (Panel 7); and endosperm cyst (Panel 8).

FIG. 27 is a copy of a photographic representation showing the interaction between FIS1 and FIS3 polypeptides in a yeast two-hybrid assay system. Left panel, formation of FIS1/FIS1 homodimers. Right panel, formation of FIS1/FIS3 heterodimers. Below, a schematic representation of the constructs used, as described in the Examples.

FIG. 28 is a copy of a photographic representation showing the interaction between FIS1, FIS2 and FIS3 polypeptides in a yeast two-hybrid assay system. Left panel, formation of FIS1/FIS2 and FIS1/FIS2 heterodimers. Right panel, formation of EzA1/FIS3 and FIS1/FIS3 heterodimers.

FIG. 29 is a copy of a photographic representation showing the relative degree of interaction between FIS1, FIS2, FIS3 and EzA1 polypeptides in a yeast two-hybrid assay system, wherein yeast growth under adenine selection requires binding between the proteins expressed from both the pGBT vector and the pGAD vector, and wherein the number of + symbols is proportional to the degree of yeast growth observed under adenine selection and “−” indicates no yeast growth. The proteins expressed from each vector are also indicated.

FIG. 30 is a copy of a schematic representation of a screening method for the isolation of MOF repressor genes that regulate FIS gene expression.

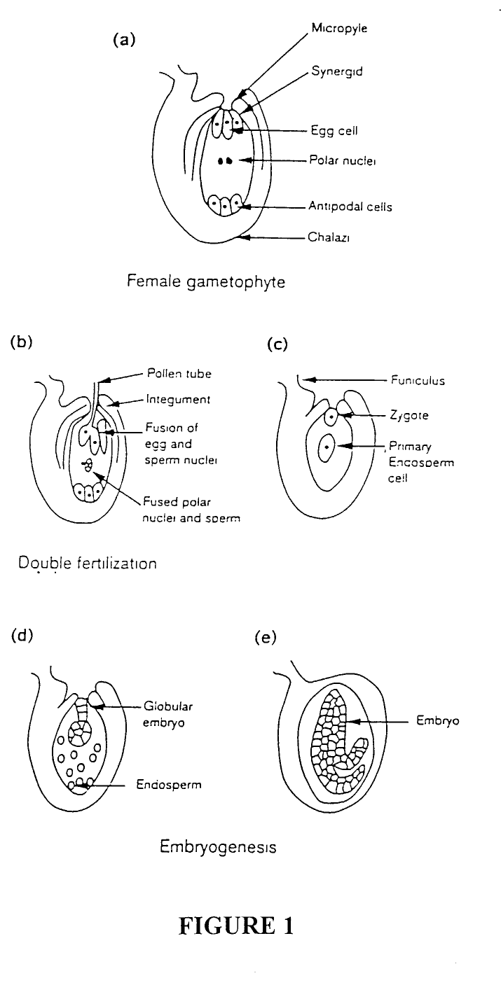

### DETAILED DESCRIPTION OF THE INVENTION

One aspect of the present invention provides a method of inducing autonomous endosperm development in a plant, said method at least comprising the step of inhibiting, interrupting or otherwise reducing the expression of a negative regulator of seed formation in one or more female reproductive cells, tissues or organs of said plant or a progenitor cell, tissue or organ thereof.

Preferably, the inventive method provides in part or whole for autonomous embryogenesis and more preferably, for autonomous seed development in plants. It this regard, it will be apparent to those skilled in the art from the description provided herein that, in order for autonomous embryogenesis or autonomous seed development to occur, the methods and reagents described herein may, in certain circumstances, represent a minimum requirement and that additional unspecified integers or steps may be required. The present invention clearly extends to the use of the specific reagents and steps described herein to produce autonomous embryogenesis and/or autonomous seed development.

The word “autonomous” as used herein means in the absence of fertilization or by the process of pseudogamy. Accordingly, the terms “autonomous endosperm development” and “autonomous embryogenesis” or similar term, shall be taken to mean endosperm development and embryogenesis respectively, in the absence of fertilization or by the process of pseudogamy.

Similarly, the term “autonomous seed development” shall be taken to refer to the development of seed independent of fertilization or by the process of pseudogamy, wherein said seed comprise one or more organs of a seed, including any one or more of female gametophyte, endosperm, embryo and a seed coat, irrespective of whether or not said seed structure is fertile or infertile. Accordingly, autonomous seed development clearly includes the process of “apomixis” wherein viable seed are produced either in the absence of fertilisation or by the process of pseudogamy. Where the production of fertile seed is required, it is essential that autonomous seed development leads to the formation of at least an endosperm and an embryo, notwithstanding that the endosperm may subsequently degenerate. In certain commercial applications involving the production of soft-seeded or parthenocarpic fruit varieties, autonomous endosperm formation may comprise the formation of non-viable seed wherein the embryo crushes down, leaving only soft seed comprising an endosperm. Alternatively, the endosperm may commence development autonomously and later degenerate, leaving seedless fruit.

In the present context, the word “seed” shall be taken to refer to any plant structure which is formed by continued differentiation of the ovule of the plant, following its normal maturation point at flower opening, irrespective of whether it is formed in the presence or absence of fertilization and irrespective of whether or not said seed structure is fertile or infertile. Fertile seeds will generally require all tissues and organs required for development of a plant, including a storage tissue such as a haploid female gametophyte or a triploid maternally-derived endosperm, an embryo and a seed coat. Infertile seeds may lack one or more of the tissues or organs present in a fertile seed and may not give rise to a plant in the next generation. It will be known to those skilled in the art that not all seed comprise an endosperm and that some angiosperm seeds comprise only an embryo and seed coat, whilst many gymnosperm seed comprise a female gametophyte as storage tissue (rather than an endosperm), in addition to a seed coat and an embryo.

The word “expression” as used herein shall be taken in its widest context to refer to the transcription of a particular genetic sequence to produce sense or antisense mRNA or the translation of a sense mRNA molecule to produce a peptide, polypeptide, oligopeptide, protein or enzyme molecule. In the case of expression comprising the production of a sense mRNA transcript, the word “expression” may also be construed to indicate the combination of transcription and translation processes, with or without subsequent post-translational events which modify the biological activity, cellular or sub-cellular localization, turnover or steady-state level of the peptide, polypeptide, oligopeptide, protein or enzyme molecule.

By “inhibiting, interrupting or otherwise reducing the expression” of a stated integer is meant that transcription and/or translation post-translational modification of the integer is inhibited or prevented or interrupted such that the specified integer has a reduced biological effect on a cell, tissue, organ or organism in which it would otherwise be expressed. Alternatively or in addition, the term “inhibiting, interrupting or otherwise reducing the expression” of a stated integer shall be taken to mean that the rate or steady-state level of transcription of the integer is reduced and/or the rate or steady-state level of translation of the integer is reduced and/or that the biological activity or steady-state level of the peptide, polypeptide, oligopeptide, protein or enzyme molecule is reduced, such that the stated integer has a reduced biological effect on a cell, tissue, organ or organism in which it would otherwise be expressed. Alternatively or in addition, the term “inhibiting, interrupting or otherwise reducing the expression” of a stated integer shall be taken to mean that a post-translational event which modifies the biological activity of the stated integer is modified such that the stated integer has a reduced biological effect on a cell, tissue, organ or organism in which it would otherwise be expressed, including a modification to the cellular or sub-cellular localization of the stated integer and/or increased turnover of the stated integer.

Those skilled in the art will be aware of how whether expression is inhibited, interrupted or reduced, without undue experimentation.

For example, the level of expression of a particular gene may be determined by polymerase chain reaction (PCR) following reverse transcription of an mRNA template molecule, essentially as described by McPherson et al. (1991). Alternatively, the expression level of a genetic sequence may be determined by northern hybridisation analysis or dot-blot hybridisation analysis or in situ hybridisation analysis or similar technique, wherein mRNA is transferred to a membrane support and hybridised to a “probe” molecule which comprises a nucleotide sequence complementary to the nucleotide sequence of the mRNA transcript encoded by the gene-of-interest, labelled with a suitable reporter molecule such as a radioactively-labelled dNTP (eg [α-³²P]dCTP or [α-³⁵S]dCTP) or biotinylated dNTP, amongst others. Expression of the gene-of-interest may then be determined by detecting the appearance of a signal produced by the reporter molecule bound to the hybridised probe molecule. Alternatively, the rate of transcription of a particular gene may be determined by nuclear run-on and/or nuclear run-off experiments, wherein nuclei are isolated from a particular cell or tissue and the rate of incorporation of rNTPs into specific mRNA molecules is determined. Alternatively, the expression of the gene-of-interest may be determined by RNase protection assay, wherein a labelled RNA probe or “riboprobe” which is complementary to the nucleotide sequence of mRNA encoded by said gene-of-interest is annealed to said mRNA for a time and under conditions sufficient for a double-stranded mRNA molecule to form, after which time the sample is subjected to digestion by RNase to remove single-stranded RNA molecules and in particular, to remove excess unhybridised riboprobe. Such approaches are described in detail by Sambrook et al. (1989) and Ausubel (1987).

Those skilled in the art will also be aware of various immunological and enzymatic methods for detecting the level of expression of a particular gene at the protein level, for example using rocket immunoelectrophoresis, ELISA, radioimmunoassay and western blot immunoelectrophoresis techniques, among others.

The term “negative regulator” shall be taken to mean any peptide, oligopeptide, polypeptide, protein, enzyme, RNA, mRNA, tRNA or DNA molecule, secondary metabolite, macromolecule or small molecule which is capable of delaying, interrupting or preventing a biological process in a cell, tissue, organ or organism.

Those skilled in the art will be aware that the term “female reproductive cells, tissues or organs” refers to cells and tissues and organs comprising the gynoecium, ovule, female gametophyte, nucellus or integument, wherein each integer is considered collectively or in isolation.

A “progenitor cell, tissue or organ” refers to a cell, tissue or organ which is capable of developing into a cell, tissue or organ which comprises a stated integer. In the present context, a progenitor cell, tissue or organ refers to a cell, tissue or organ which is capable of developing into a female reproductive cell, tissue or organ as defined herein.

Accordingly, the term “negative regulator of seed formation” refers to a peptide, oligopeptide, polypeptide, protein, enzyme, RNA, mRNA, tRNA or DNA molecule, secondary metabolite, macromolecule or small molecule which is capable of delaying, interrupting or preventing the formation of seed or a seed organ in a plant. With particular reference to the presently described invention, a “negative regulator of seed formation” refers to any peptide, oligopeptide, polypeptide, protein, enzyme, RNA, mRNA, tRNA or DNA molecule, secondary metabolite, macromolecule or small molecule which is capable of delaying, interrupting or preventing autonomous endosperm development in a plant.

Preferred negative regulators of seed formation in the present context are peptides, oligopeptides, polypeptides, proteins or enzymes which are capable of delaying, interrupting or preventing autonomous seed development in a plant. Such negative regulators may be repressors of one or more steps in autonomous (i.e. fertilization-independent) seed development in the plant.

For the purposes of nomenclature, the terms “fertilisation-independent seed gene product”, “FIS gene product”, “FIS protein”, “FIS polypeptide” or “FIS peptide” or similar term shall be used to refer to a negative regulator of seed formation. The term “FIS gene” shall be taken to refer to the gene which encodes such a negative regulator of seed formation. In this context, specific FIS peptides, FIS polypeptides, FIS proteins and FIS genes are referred to by numerical descriptors, as are the alleles of such peptides, polypeptides, proteins and genes. For example, the FIS genes are described herein as FIS1, FIS2 and FIS3, etc., whilst the allelic variants at each gene locus are referred to as FIS1-1, FIS1-2, FIS1-3, FIS2-1, FIS2-2, FIS3-3, etc.

As will be known to those skilled in the art, mutated forms of a specific wild-type FIS gene product or gene encoding same, are indicated herein in lower case, for example as fis1 polypeptide, fis1 gene, etc.

Without being bound by any theory or mode of action, such negative regulators may, when expressed in the plant, prevent autonomous endosperm development from being initiated or alternatively, prevent autonomous endosperm development from progressing once it has been initiated, thereby optionally promoting a “default” pathway wherein seed comprising an endosperm are produced by sexual means via fertilization. Negative regulators of autonomous endosperm formation are also most likely to be expressed normally in maternally-derived cells, tissues and organs of the plant, because an implicit feature of autonomous endosperm development is the absence of a genetic contribution from the male gametophyte. Additionally, as exemplified herein, plants in which the expression of one or more negative regulators of autonomous endosperm development has been prevented or reduced in the maternal tissues are capable of reproducing sexually in the presence of a pollen donor, indicating that the negative regulator is not derived from the male gametophyte.

Accordingly, in a preferred embodiment, the negative regulator of seed formation is a peptide, polypeptide or protein which, when expressed in maternal tissues of a plant, completely or partially inhibits or prevents the autonomous development of the ovule into a seed (i.e. it prevents or at least reduces the frequency fertilization-independent seed development) and more preferably, a peptide, polypeptide or protein which, when expressed in maternal tissues of a plant, completely or partially inhibits or prevents autonomous embryogenesis and/or partial autonomous endosperm development in the plant.

A particularly preferred embodiment of the present invention provides a method of inducing autonomous endosperm development in a plant, said method at least comprising the step of inhibiting, interrupting or otherwise reducing the expression of a negative regulator of seed formation in one or more female reproductive cells, tissues or organs of said plant or a progenitor cell, tissue or organ thereof, wherein the negative regulator of seed formation is a FIS polypeptide selected from the list comprising:

(i) a FIS1 polypeptide which comprises an amino acid sequence having at least about 50% overall amino acid sequence identity to the amino acid sequence set forth in SEQ ID NO:1;

(ii) a FIS2 polypeptide which comprises an amino acid sequence having at least about 60-70% amino acid sequence identity to the amino acid sequence set forth in SEQ ID NO:2;

(iii) a FIS3 polypeptide which comprises an amino acid sequence having at least about 60-70% amino acid sequence identity to the amino acid sequence set forth in SEQ ID NO:3; and

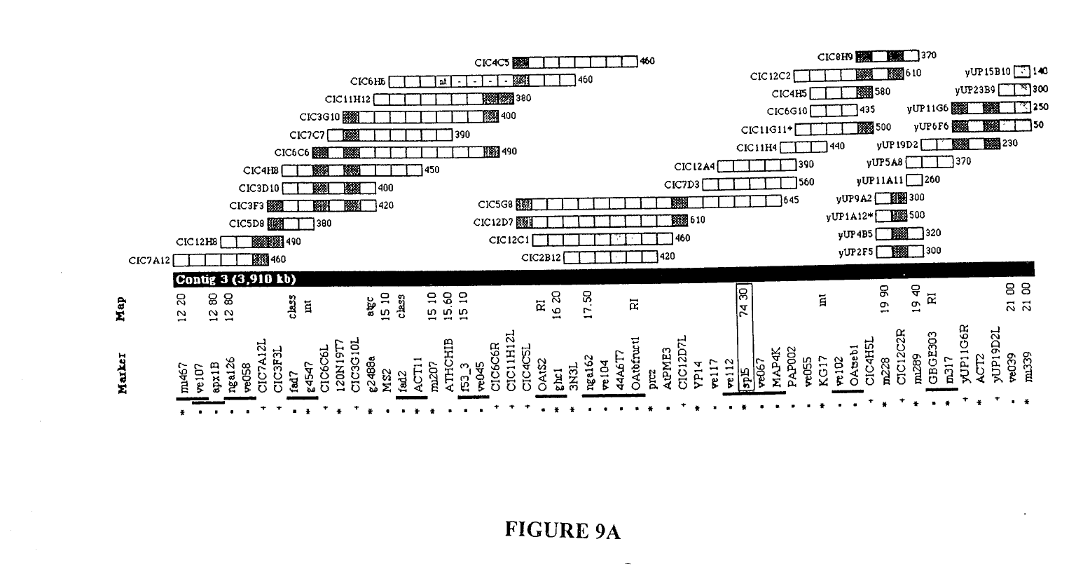

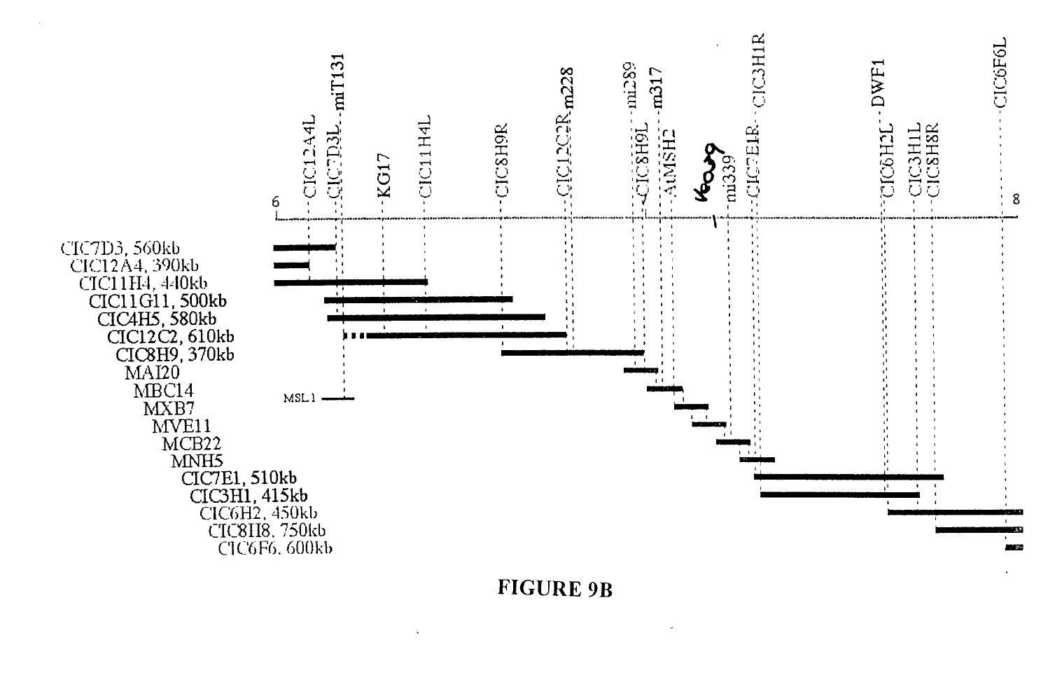

(iv) a FIS3 polypeptide encoded by a nucleotide sequence which is capable of hybridizing under at least low stringency conditions to that region of chromosome 3 of Arabidopsis thaliana which maps between the markers m317 and DWF1 as set forth in FIG. 9B.

Preferably, a FIS1 polypeptide which is at least 50% identical to the amino acid sequence set forth in SEQ ID NO:1 further comprises:

(i) a cysteine-rich domain designated C5, comprising the consensus amino acid sequence motif:

C-X₂-C-X₄-C-X₂₅₋₃₅-C-X₃-C, (as represented herein by the individual sequences set forth in SEQ ID NO:10 through SEQ ID NO:20),wherein numerical values indicate the number of consecutive multiple occurrences of a particular amino acid residue;

(ii) a cysteine-rich domain designated the CXC domain which comprises at least about 14 cysteine residues within a sequence of 61-67 consecutive amino acids and located C-terminal to the C5 domain; and

(iii) a consensus amino acid sequence motif designated SET and located C-terminal to the CXC domain and comprising the amino acid sequence:

S-(D/K)-(I/V)-X-G-X-G-X-F-X₆-K-X-E-(Y/F)-(L/I)-X-E-Y-(T/C)-G-E-X-I-(T/S)-X₂-E-(A/D)-X₂-R-G-X-(I/V)-(E/Y)-D-(R/K)-X₂-(C/S)-S-(F/Y)-(L/I)-F-X-(L/I)-X₆-D-X₂-(R/K)-(K/I)-G-(N/D)-X₂-(K/R)-F-X-N-H-X₃₋₄-P-X-C-Y-A-(K/R)-X-(M/I)-X-V-X-G-(D/E)-(H/Q)-R-(I/V)-G-X-(F/Y)-A-X-(E/R)-(A/R)-(I/L)-X₂-(G/S)-E-E-L-X-F-D-Y-X-Y, (as represented herein by the individual sequences set forth in SEQ ID NO:21 to SEQ ID NO:22), wherein numerical values indicate the number of consecutive multiple occurrences of a particular amino acid residue.

More preferably, the C5 domain comprises the amino acid sequence:

C-X₂-C-X₄-C-X₂-H-X₂₂₋₃₂-C-X₃-C-(W/Y), (as represented herein by the individual sequences set forth in SEQ ID NO:23 to SEQ ID NO:33), and more preferably, the amino acid sequence

C-R-R-C-X₂-(F/Y)-D-C-X-(M/L)-H-X₂₂₋₃₂-C-X₃-C-Y, (as represented herein by the individual sequences set forth in SEQ ID NO:34 to SEQ ID NO:44) and still more preferably the amino acid sequence

C-R-R-C-X₂-F-D-C-X-M-H-X₂₂₋₃₂-C-X₃-C-Y, (as represented herein by the individual sequences set forth in SEQ ID NO:45 through SEQ ID NO:55) or a homologue, analogue or derivative of said amino acid sequence or a fragment comprising at least 5 contiguous amino acids thereof wherein numerical values indicate the number of consecutive multiple occurrences of a particular amino acid residue.

In a most particularly preferred embodiment, a FIS1 polypeptide will comprise a C5 domain having an amino acid sequence which corresponds to amino acid residues 269-309 of SEQ ID NO:1 or a homologue, analogue or derivative of said amino acid sequence.

More preferably, the cysteine-rich domain designated CXC comprises the consensus amino acid sequence,

C-X₆₋₁₀-C-X-C-X₉₋₁₀-C-X-C-X₃-C-X₆-C-X-C-X₃₋₄-C-X₄-C-X-C-X₆-C-X₄-C-X₂-C (as represented herein by the individual sequences set forth in SEQ ID NO:56 to SEQ ID NO:75) and more preferably the amino acid sequence,

C-X₆₋₁₀-C-X-C-X₉₋₁₀-C-X-C-X₃-C-X₂-R-F-X-G-C-X-C-X₂₋₃-Q-C-X₄-C-X-C-(F/Y)-X-A-X₂-E-C-(N/D)-P-X₂-C-D-X-C (as represented herein by the individual sequences set forth in SEQ ID NO:76 to SEQ ID NO:95) and still more preferably, the amino acid sequence,

C-X₆₋₁₀-C-X-C-X₉₋₁₀-C-X-C-X₃-C-X₂-R-F-X-G-C-X-C-X₂₋₃-Q-C-X₄-C-X-C-F-X-A-X₂-E-C-D-P-X₂-C-D-X-C (as represented herein by the individual sequences set forth in SEQ ID NO:96 through SEQ ID NO:115)

or a homologue, analogue or derivative of said amino acid sequence or a fragment comprising at least 5 contiguous amino acids thereof, wherein numerical values indicate the number of consecutive multiple occurrences of a particular amino acid residue.

In a most particularly preferred embodiment, a FIS1 polypeptide will comprise a CXC domain which comprises an amino acid sequence which corresponds to amino acid residues 450-515 of SEQ ID NO:1 or a homologue, analogue or derivative of said amino acid sequence.

Preferably, the SET domain will comprise a sequence of amino acids which is at least about 50-60% identical to amino acid residues 551-665 of SEQ ID NO:1, more preferably at least about 60-70% identical to amino acid residues 551-665 of SEQ ID NO:1 and still more preferably at least about 70-80% identical to amino acid residues 551-665 of SEQ ID NO:1. In a particularly preferred embodiment, the SET domain of a FIS1 polypeptide will comprise an amino acid sequence which is substantially identical or identical to amino acid residues 551-665 of SEQ ID NO:1 or a homologue, analogue or derivative of said amino acid sequence.

Alternatively or in addition, the FIS1 polypeptide will further comprise a cysteine-rich domain designated TGNF/NGFR which comprises the consensus amino acid sequence motif Cₐ-X₁₁₋₁₄-C_{b}-X₁₋₂-C_{c}-X₂₋₃-C_{d}-X₈₋₁₁-Cₑ-X₇₋₉-C_{f} (as represented herein by individual sequences set forth in SEQ ID NO:116 through SEQ ID NO:180), wherein Cₐ,C_{b},C_{c},C_{d},Cₑ and C_{f} represent successive cysteine residues in said sequence motif and numerical values indicate the number of consecutive multiple occurrences of a particular amino acid residue.

The TGNF/NGFR domain set forth in any one of SEQ ID NO:116 to SEQ ID NO:180 may include an additional one or two or three amino acids immediately before the C-terminal Cysteine residue.

Preferably, the TGNF/NGFR domain set forth in any one of SEQ ID NO:116 to SEQ ID NO:180, with or without additional C-terminal residues referred to supra, comprises Phenylalanine or Tyrosine or Histidine at position six from the N-terminus. Alternatively or in addition, the TGNF/NGFR domain set forth in any one of SEQ ID NO:116 to SEQ ID NO:180, with or without additional C-terminal residues referred to supra, comprises Glutamine or Asparagine or Aspartate or Serine in the third-to-last amino acid position of said consensus. Even more preferably, the TGNF/NGFR domain set forth in any one of SEQ ID NO:116 to SEQ ID NO:180, with or without additional C-terminal residues referred to supra, will comprise a Histidine residue at position six from the N-terminus and an Asparagine residue in the third-to-last amino acid position of said consensus (i.e. three amino acids from the C-terminus).

In a particularly preferred embodiment, the TGNF/NGFR domain comprises an amino acid sequence which corresponds to amino acid residues 460-498 of SEQ ID NO:1 or a homologue, analogue or derivative thereof.

In a further embodiment, the cysteine-rich domain designated TGNF/NGFR may further be capable of forming the intrachain disulfide bonds Cₐ-C_{b} and/or C_{c}-Cₑ and/or C_{d}-C_{f}.

In a still further embodiment, the TGNF/NGFR domain may be contained within the CXC domain of a FIS1 polypeptide, such as in the case of the Arabidopsis thaliana FIS1 polypeptide exemplified herein as SEQ ID NO:1.

Alternatively or in addition, the FIS1 polypeptide, and more particularly the SET domain of the FIS1 polypeptide, may further comprise the amino acid sequence motif R-G-D. Those skilled in the art will be aware of the structure of the R-G-D motif and its occurrence in proteins which are involved in cell adhesion (Ruoslahti and Piersbacher, 1986; d'Souza et al., 1991). Without being bound by any theory or mode of action, the tripeptide motif R-G-D (SEQ ID NO:181) may play a role in binding of the FIS1 polypeptide to a cognate receptor molecule, thereby modulating or initiating a signal transduction pathway which is relevant to autonomous seed development. For example, it is possible that the FIS1 polypeptide binds to its cognate receptor to inhibit binding of an activator molecule thereto, wherein said activator molecule would, if bound to the receptor, activate autonomous seed development in the maternal tissues. Alternatively or in addition, a FIS1 polypeptide which is at least 50% identical to the amino acid sequence set forth in SEQ ID NO:1 further comprises an amino acid sequence comprising 12-13 amino acid residues wherein at least about 5-12 of said residues, more preferably at least about 8-12 of said residues, are the acidic amino acids glutamate and/or aspartate. In an even more preferred embodiment, at least 12 of the amino acids in the 12-13 amino acid long sequence will be acidic residues. In a particularly preferred embodiment, the FIS1 polypeptide will comprise the amino acid sequence set forth in SEQ ID NO:182 as follows:

E-E-D-E-E-D-E-E-E-D-E-E-E,

or a homologue, analogue or derivative of said amino acid sequence. According to this embodiment, it is particularly preferred that the acidic domain is located in the N-terminal region of the FIS1 polypeptide, more preferably N-terminal to the C5 domain. While not being bound by any theory or mode of action, this acidic region may be required for forming an interaction with other proteins.

Alternatively or in addition, a FIS1 polypeptide which is at least 50% identical to the amino acid sequence set forth in SEQ ID NO:1 further comprises an amino sequence which is at least about 50% identical to the consensus amino acid sequence motif set forth in SEQ ID NO:183, and designated “WCA motif” as follows:

W-X-(P/R/G)-X-(E/A/D)-X₂-(L/M)-(Y/F/M)-X-(K/S/V)-(G/M/L)-X-(E/K/G)-I-F-G-X-N-S-C-X-(I/V)-A-X-(N/H)-(L/I/M)-(L/M)-X-G-X-K-(T/S)-C,

or alternatively (SEQ ID NO:184 to SEQ ID NO:186),

W-X-(P/G)-X-(E/D)-X₂-(L/M)-(Y/F)-X-(K/V)-(G/L)-X₃-(F/Y)-(G/L)-X-N-X-C-X-(I/V)-A-X-(N/L)-(L/I/M)-(L/G)-X₁₋₃

-K-(T/S)-C

and more preferably the amino acid sequence set forth in SEQ ID NO:187, as follows:

W-X-P-X-E-K-X-L-Y-L-K-G-X-E-I-F-G-X-N-S-C-X-(I/V)-A-X-N-I-L-X-G-X-K-T-C,

and even more preferably the amino acid sequence set forth in SEQ ID NO:188, as follows:

W-X-P-X-E-K-X-L-Y-L-K-G-X-E-I-F-G-X-N-S-C-X-V-A-X-N-I-L-X-G-X-K-T-C,

or a homologue, analogue or derivative of said amino acid sequence or a fragment comprising at least 5 contiguous amino acids thereof located C-terminal to the C5 domain and N-terminal to the CXC domain, subject to the proviso that the first cysteine residue and the alanine residue are always present, the amino acid residue at position 1 in said consensus is a hydrophobic amino acid residue and the amino acid residue at positions 27 and 28 in said consensus is either L or M.

In a particularly preferred embodiment, the FIS1 polypeptide will further comprise a WCA motif which comprises the amino acid sequence set forth in SEQ ID NO:189, as follows:

W-T-P-V-E-K-D-L-Y-L-K-G-I-E-I-F-G-R-N-S-C-D-V-A-L-N-I-L-R-G-L-K-T-C,

or a homologue, analogue or derivative of said amino acid sequence or a fragment comprising at least 5 contiguous amino acids thereof located C-terminal to the C5 domain and N-terminal to the CXC domain.

Optionally, the FIS1 polypeptide further comprises a nuclear localisation domain located C-terminal to the C5 domain and N-terminal to the CXC domain. As used herein, the term “nuclear localisation domain” shall be taken to refer to an amino acid sequence which is at least postulated to be capable of targeting a polypeptide comprising said domain to the nucleus of a cell. Those skilled in the art will be aware of the specific requirements of a domain which is postulated to be involved in nuclear localisation. Preferably, a nuclear localisation domain comprises an amino acid sequence which is rich in lysine and/or arginine residues. More preferably, the nuclear localisation signal of a FIS1 polypeptide will include the amino acid sequence motif set forth in SEQ ID NO:190 to SEQ ID NO:191, as follows:

K-K-X₁₋₂-(R/K)-K

and more preferably, the amino acid sequence set forth in SEQ ID NO:192 to SEQ ID NO:193, as follows:

K-K-X-₁₋₂-(R/K)-K-X₂-R-X₂-R-K-K-X-R-X-R-K

and still more preferably,the amino acid sequence set forth in SEQ ID NO:193, as follows:

K-K-X₂-(R/K)-K-X₂-R-X₂-R-K-K-X-R-X-R-K

or a homologue, analogue or derivative of said amino acid sequence or a fragment comprising at least 5 contiguous amino acids thereof, wherein numerical values indicate the number of consecutive multiple occurrences of a particular amino acid residue.

In a particularly preferred embodiment, the nuclear localisation signal of a FIS1 polypeptide will include the amino acid sequence motif set forth in SEQ ID NO:194, as follows:

K-K-V-S-R-K-S-S-R-S-V-R-K-K-S-R-L-R-K

or a homologue, analogue or derivative of said amino acid sequence or a fragment comprising at least 5 contiguous amino acids thereof which retains the potential to target a polypeptide to the nucleus.

In a particularly preferred embodiment of the invention, a FIS1 polypeptide having at least about 50% amino acid sequence identity to the amino acid sequence set forth in SEQ ID NO:1 will further comprise all of the amino acid sequence motifs and protein domains described supra.

For the purposes of further describing the FIS1 polypeptide, it is preferred that the percentage identity to the amino acid sequences set forth in SEQ ID NO:1 is at least about 60-70% overall, more preferably at least about 70-80% overall, still more preferably at least about 80-90% overall and still even more preferably at least about 90-99% identity overall. In a particularly preferred embodiment, the negative regulator of seed formation will comprise an amino acid sequence sharing absolute identity to the amino acid sequence set forth in SEQ ID NO:1 or a homologue, analogue or derivative of said amino acid sequence.

For the purposes of nomenclature, the amino acid sequence set forth in SEQ ID NO:1 is a polycomb protein (Goodrich et al., 1997) having homology to the Enhancer of zeste [E(z)] family of proteins (Laible et al.(1997), which was derived from Arabidopsis thaliana and described initially by Grossniklaus et al. (1998). Those skilled in the art will be aware of the structure and function of the polycomb group of proteins and in particular, the E(z) class of proteins. By way of background, the E(z) proteins generally comprise a SET-like domain, in addition to a CXC-like domain and a C5-like domain.

Whilst not being bound by any theory or mode of action, proteins which contain a SET domain are generally involved in regulating gene expression by controlling chromatin structure and thereby modulating the accessibility of the chromatin to transcription factors. The C5 domain and CXC domain appear to be necessary for the function of the Drosophila E(z) polypeptide, which also comprises a SET domain. Accordingly, the possibility exists that the FIS1 polypeptide may interact with nuclear chromatin to prevent positive regulatory factors which would otherwise be capable of inducing autonomous seed development and/or partial autonomous endosperm development and/or autonomous embryogenesis from interacting with the chromatin and inducing such autonomous developmental patterns.

For the present purpose of inducing autonomous seed development, the step of inhibiting, interrupting or otherwise reducing the expression of the FIS1 polypeptide in one or more female reproductive cells, tissues or organs of said plant or a progenitor cell, tissue or organ thereof, requires more than the mere disruption of the SET domain present in said protein. In this regard, Grossniklaus et al. (1998) demonstrated that a mutation in nucleotide sequence encoding the FIS1 polypeptide, known as medea (mea), produces 50% embryo lethality in the seed produced following self-fertilization of MEA/mea plants (i.e. plants which are heterozygous for the mutant allele), however these authors did not demonstrate autonomous seed development and/or partial autonomous endosperm development and/or autonomous embryogenesis. The mea mutant allele at this locus comprises a Ds transposable element inserted within or N-terminal to the SET domain of FIS1 which is present in the E(z) protein family, thereby resulting in the translation of a fis1 mutant polypeptide designated medea (mea) which lacks the SET domain, however comprises all protein domains N-terminal to the site of insertion of Ds.

Accordingly, this aspect of the invention, in so far as it relates to the inhibition, interruption or reduction in expression of a negative regulator of seed formation which comprises the amino acid sequence set forth in SEQ ID NO:1, does not exclusively utilise the mutation or disruption of the SET domain of SEQ ID NO:1 (i.e. amino acid residues 551 to 665 of SEQ ID NO:1) or the mimicking the mea mutant allele. Such exclusive mutation or disruption of the SET domain does not, in any event, produce a plant which is capable of autonomous seed formation, autonomous embryogenesis or autonomous endosperm development.

As exemplified herein, the present inventors have discovered that mutations in the FISI gene which eliminate one or more of the amino acid sequences upstream of the SET domain and optionally including the SET domain are capable of conferring autonomous seed formation on plants.

Accordingly, in performing the present invention, the expression of the FIS1 polypeptide may be inhibited, disrupted, prevented or otherwise reduced by preventing the synthesis of a polypeptide which comprises any one or more of the FIS1 protein domains or amino acid sequence motifs described herein, subject to the proviso that said FIS1 protein domain or amino acid sequence motif does not comprise exclusively the SET domain.

Accordingly, the present invention clearly encompasses the mutation or disruption of the SET domain of SEQ ID NO:1 in conjunction with other means for inhibiting, interrupting or otherwise reducing the expression of the amino acid sequence set forth in SEQ ID NO:1, for example the mutation or disruption of one or more other regions of said amino acid sequence, the only requirement being that said other means produces a plant which is capable of autonomous seed formation, autonomous embryogenesis or autonomous endosperm development.

In a particularly preferred embodiment, all of the FIS1 protein domains are prevented from being expressed in the performance of the invention, including the production of a null allele.

For the purposes of nomenclature, the amino acid sequence set forth in SEQ ID NO:2 relates to the Arabidopsis thaliana FIS2 polypeptide, a putative C2H2 zinc-finger protein or zinc-finger-like protein which is involved in regulating autonomous embryogenesis and partially-regulating autonomous endosperm development, at least in that plant.

Accordingly, it is particularly preferred that a FIS2 polypeptide which is at least about 50% identical to the amino acid sequence set forth in SEQ ID NO:2 will further comprise a zinc-finger protein motif or zinc-finger-like protein motif which comprises about 20 to about 25 amino acid residues in length, containing the amino acid sequence motifs set forth in SEQ ID NO:195 and SEQ ID NO:196, as follows:

SEQ ID NO:195: C-X₂-C-X; and

SEQ ID NO:196: X-H-X₄-H.

More preferably, a FIS2 polypeptide will comprise a zinc-finger protein motif or zinc-finger-like protein motif which comprises the amino acid sequence set forth in SEQ ID NO:197, as follows:

C-X₂-C-X₆-H-X₅-H-X₄-H,

and even more particularly, the amino acid sequence set forth in SEQ ID NO:198, as follows:

C-X₂-C-X₃-C-X₂-H-X₅-H-X₄-H.

In a more particularly preferred embodiment, a FIS2 polypeptide will comprise a zinc-finger protein motif or zinc-finger-like protein motif which comprises the amino acid sequence set forth in SEQ ID NO:199, as follows:

(i) C-P-F-C-L-I-P-C-G-G-H-E-G-L-Q-L-H-L-K-S-S-H; or

(ii) a homologue, analogue or derivative of said amino acid sequence.

As used herein, the term “zinc-finger protein motif” shall be taken to refer to a primary amino acid sequence which is capable of forming a secondary protein structure which is characteristic of the class of transcription factors known in the art as “zinc-finger” proteins, wherein said secondary protein structure is formed by the formation of disulfide bridges between cysteine residues in the primary amino acid sequence.

The term “zinc-finger-like protein motif” shall be taken to refer to a primary amino acid sequence which shows amino acid sequence similarity to a zinc-finger protein motif, notwithstanding that it is not capable of forming a secondary protein structure characteristic of zinc-finger proteins by the formation of disulfide bridges between cysteine residues in the primary amino acid sequence.

For the purposes of further describing the FIS2 polypeptide, it is preferred that the percentage identity to the amino acid sequences set forth in SEQ ID NO:2 is at least about 60-70% overall, more preferably at least about 70-80% overall, still more preferably at least about 80-90% overall and still even more preferably at least about 90-99% identity overall. In a particularly preferred embodiment, the negative regulator of seed formation will comprise an amino acid sequence sharing absolute identity to the amino acid sequences set forth in SEQ ID NO:2 or a homologue, analogue or derivative thereof.

For the purposes of nomenclature, the amino acid sequence set forth in SEQ ID NO:3 relates to the Arabidopsis thaliana FIS3 polypeptide, a protein which is involved in regulating autonomous endosperm development, at least in that plant.

For the purposes of further describing the FIS3 polypeptide, it is preferred that the percentage identity to the amino acid sequence set forth in SEQ ID NO:3 is at least about 60-70% overall, more preferably at least about 70-80% overall, still more preferably at least about 80-90% overall and still even more preferably at least about 90-99% identity overall. In a particularly preferred embodiment, the negative regulator of seed formation will comprise an amino acid sequence sharing absolute identity to the amino acid sequences set forth in SEQ ID NO:3 or a homologue, analogue or derivative thereof.

In an alternative embodiment, the FIS3 polypeptide will be encoded by a nucleic acid moelcule that is capable of hybridising under at least low stringency hybridisation conditions to the fis3 mutant allele.

As exemplified herein, the present inventors have identified a mutant phenotype designated fis3 which is at least capable of autonomous endosperm development and/or autonomous seed formation. The present inventors have mapped the fis3 mutant allele to chromosome 3 of Arabidopsis thaliana, at a region which lies between the morphological markers hy3 and gl1. Further mapping localized the fis3 mutant allele to a region between the RFLP markers m317 and DWF1. The fis3 allele has been shown further to map to a region on chromosome 3 of A. thaliana which is approximately 6 cM from the SSLP marker nga162 and approximately 1 cM from the RFLP marker ve039.

Those skilled in the art will be aware that the close genetic linkage between the FIS3 locus on chromosome 3 of A. thaliana and the RFLP marker ve039 indicates that said RFLP marker is useful in identifying plants which comprise the FIS3 gene and in isolating the FIS3 gene.

Accordingly, it is preferred that a FIS3 polypeptide will be encoded by a nucleotide sequence which is capable of hybridizing under at least low stringency conditions to the RFLP marker designated ve039 which maps approximately 1 cM from the FIS3 locus on chromosome 3 of Arabidopsis thaliana.

For the purposes of defining the stringency, a low stringency is defined herein as being a hybridisation and/or a wash carried out in 6×SSC buffer, 0.1% (w/v) SDS at 28° C. Generally, the stringency is increased by reducing the concentration of SSC buffer, and/or increasing the concentration of SDS and/or increasing the temperature of the hybridisation and/or wash. Conditions for hybridisations and washes are well understood by one normally skilled in the art. For the purposes of clarification of parameters affecting hybridisation between nucleic acid molecules, reference can conveniently be made to pages 2.10.8 to 2.10.16 of Ausubel et al. (1987), which is herein incorporated by reference.

Those skilled in the art will be aware that confirmation of the identity of the FIS3 gene may be carried out by complementation of the fis3 mutant phenotype using YAC, BAC or cosmid clones or fragments thereof which hybridize to the RFLP marker ve039. The nucleotide sequence of the FIS3 gene may then be determined by sequencing the genes present in those clones which successfully complement the fis3 mutant phenotype.

Accordingly, the present inventors have further created a map of contiguous YAC and p1 cosmid clones in the region surrounding the RFLP marker ve039, which indicates that the fis3 mutant allele (and thus the wild-type FIS3 gene) is localized on the YACS and/or p1 clones MCB22 and/or MNH5 and/or CIC7E1.

Accordingly, in a further preferred embodiment of the invention the FIS3 polypeptide is encoded by a nucleic acid molecule which is capable of hybridising under at least low stringency hybridisation conditions to one or more of the YACS and/or p1 clones designated MCB22 and/or MNH5 and/or CIC7E1.

For the purposes of nomenclature, the RFLP marker ve039 and the YAC clone CIC7E1 and the p1 clones MCB22 and MNH5 are all publicly available from the following internet sites: http://www.Kazusa.or.JP/arabi/chr3/ and http://genome-www.stanford.edu/Arabidopsis/chr3-INRA/

More preferably, FIS3-encoding genetic sequences are preferably isolated by hybridisation under medium or more preferably, under high stringency conditions, to a probe which comprises at least about 30 contiguous nucleotides derived from the region of chromosome 3 of Arabidopsis thaliana which maps between the markers m317 and DWF1 as set forth in FIG. 9B.

It will be apparent from the preceding description that the present invention clearly extends to the modulation of expression of negative regulators of seed development which comprise homologues, analogues and derivatives of a FIS polypeptide, including the FIS1 and FIS2 amino acid sequences set forth in SEQ ID NO:1 and SEQ ID NO:2 respectively, and the FIS3 polypeptide encoded by a nucleotide sequence which is capable of hybridizing under at least low stringency conditions to that region of chromosome 3 of Arabidopsis thaliana which maps between the markers m317 and DWF1.

In the present context, “homologues” of a FIS polypeptide refer to those amino acid sequences or peptide sequences which are derived from polypeptides, enzymes or proteins of the present invention or alternatively, correspond substantially to the polypeptides and amino acid sequences listed supra, notwithstanding any naturally-occurring amino acid substitutions, additions or deletions thereto.

For example, amino acids may be replaced by other amino acids having similar properties, for example hydrophobicity, hydrophilicity, hydrophobic moment, antigenicity, propensity to form or break α-helical structures or β-sheet structures, and so on. Alternatively, or in addition, the amino acids of a homologous amino acid sequence may be replaced by other amino acids having similar properties, for example hydrophobicity, hydrophilicity, hydrophobic moment, charge or antigenicity, and so on.

Naturally-occurring amino acid residues contemplated herein are described in Table 1. A homologue may be a synthetic peptide produced by any method known to those skilled in the art, such as by using Fmoc chemistry.

Alternatively, a homologue of a FIS polypeptide may be derived from a natural source, such as the same or another species as the polypeptides, enzymes or proteins of the present invention. Preferred sources of homologues of the amino acid sequences listed supra include any of the sources contemplated herein.

“Analogues” of a FIS polypeptide encompass those amino acid sequences which are substantially identical to the amino acid sequences listed supra notwithstanding the occurrence of any non-naturally occurring amino acid analogues therein.

Preferred non-naturally occurring amino acids contemplated herein are listed below in Table 2.

The term “derivative” in relation to a FIS polypeptide shall be taken to refer hereinafter to mutants, parts, fragments or polypeptide fusions of said polypeptides. Derivatives include modified amino acid sequences or peptides in which ligands are attached to one or more of the amino acid residues contained therein, such as carbohydrates, enzymes, proteins, polypeptides or reporter molecules such as radionuclides or fluorescent compounds. Glycosylated, fluorescent, acylated or alkylated forms of the subject peptides are also contemplated by the present invention. Additionally, derivatives may comprise fragments or parts of an amino acid sequence disclosed herein and are within the scope of the invention, as are homopolymers or heteropolymers comprising two or more copies of the subject sequences.

Procedures for derivatizing peptides are well-known in the art.

Substitutions encompass amino acid alterations in which an amino acid is replaced with a different naturally-occurring or a non-conventional amino acid residue. Such substitutions may be classified as “conservative”, in which case an amino acid residue is replaced with another naturally-occurring amino acid of similar character, for example Gly⇄Ala, Val⇄Ile⇄Leu, Asp⇄Glu, Lys⇄Arg, Asn⇄Gln or Phe⇄Trp⇄Tyr. Substitutions encompassed by the present invention may also be “non-conservative”, in which an amino acid residue which is present in a repressor polypeptide is substituted with an amino acid having different properties, such as a naturally-occurring amino acid from a different group (eg. substituted a charged or hydrophobic amino acid with alanine), or alternatively, in which a naturally-occurring amino acid is substituted with a non-conventional amino acid.

Amino acid substitutions are typically of single residues, but may be of multiple residues, either clustered or dispersed.

Amino acid deletions will usually be of the order of about 1-10 amino acid residues, while insertions may be of any length. Deletions and insertions may be made to the N-terminus, the C-terminus or be internal deletions or insertions. Generally, insertions within the amino acid sequence will be smaller than amino-or carboxyl-terminal fusions and of the order of 1-4 amino acid residues.

Preferred homologues, analogues and derivatives of the FIS polypeptides described herein, including the amino acid sequences set forth in SEQ ID NO:1 and/or SEQ ID NO:2 and/or SEQ ID NO:3, will comprise at least about 5-10 contiguous amino acids of said polypeptide or preferably at least about 10-20 contiguous amino acid residues or more preferably at least about 20-50 contiguous amino acid residues. Accordingly, such homologues, analogues and derivatives may be full-length or less than full-length sequences compared to the full-length A. thaliana FIS polypeptides.

It will be apparent to those skilled in the art that the expression of a homologue, analogue or derivative of a FIS polypeptide which is targeted (i.e. prevented, interrupted or otherwise reduced) using the inventive method described herein must be capable of functioning in vivo as a negative regulator of seed development in a plant and preferably in the maternal cells, tissues or organs thereof.

In other embodiments of the invention described herein, homologues, analogues and derivatives of a FIS polypeptide may be useful as a tool in performing the inventive method. For example, homologues, analogues and derivatives of the FIS polypeptide, including those which are shorter than the full-length sequence and do not possess the same activity as the full-length sequence, will at least be useful in the preparation of antibody molecules capable of binding to the full-length sequence for use in diagnostic assays or as inhibitor molecules. Alternatively such homologues, analogues and derivatives may be useful as inhibitors of the full-length FIS1 and/or FIS2 and/or FIS3 polypeptides, by preventing binding of the full-length polypeptides to a protein or nucleic acid molecule with which they interact in vivo. For example, homologues, analogues or derivatives of the FIS2 polypeptide may comprise the zinc-finger motif and act as a non-functional competitive inhibitor of the full-length polypeptide.

Alternatively or in addition, a homologue, analogue or derivative of the FIS polypeptides described herein will be catalytically equivalent to the naturally-occurring FIS polypeptide exemplified herein and comprise an amino acid sequence which is at least about 60-70% identical thereto. Preferably, the percentage identity to SEQ ID NO:2 will be at least about 70-80%, more preferably at least about 80-90% and even more preferably at least about 90-95% or at least about 98 or 99%.

In determining whether or not two amino acid sequences fall within defined percentage identity or similarity limits, those skilled in the art will be aware that it is necessary to conduct a side-by-side comparison of amino acid sequences. In such comparisons or alignments, differences will arise in the positioning of non-identical amino acid residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using the GAP programme of the Computer Genetics Group, Inc., University Research Park, Madison, Wis., United States of America (Devereaux et al, 1984), which utilizes the algorithm of Needleman and Wunsch (1970) or alternatively, the CLUSTAL W algorithm of Thompson et al (1994) for multiple alignments, to maximise the number of identical/similar amino acids and to minimise the number and/or length of sequence gaps in the alignment.

Means for inhibiting, interrupting or otherwise reducing the expression of a negative regulator of seed formation in one or more female reproductive cells, tissues or organs of a plant or a progenitor cell, tissue or organ thereof include any means known to those skilled in the art in so far as said means are applicable to the FIS polypeptides described herein or a homologue, analogue or derivative thereof.

Such means include mutagenesis of the gene(s) which encode(s) the FIS polypeptide(s) described herein, such that it is no longer capable of being expressed at a biologically-effective level in the maternal cells, tissues or organs of the plant. Means for performing such mutagenesis of a FIS gene include the use of chemical mutagens, radiation and insertional inactivation by molecular means, amongst others and the present invention clearly encompasses the use of all such methods.

As used herein, the term “biologically-effective level” shall be taken to mean a level of expression of a FIS polypeptide which is sufficient to delay, inhibit, interrupt or prevent autonomous seed development and/or partial autonomous endosperm development and/or autonomous embryogenesis in a plant.

Reference herein to a “gene” is to be taken in its broadest context and includes:

(i) a classical genomic gene consisting of transcriptional and/or translational regulatory sequences and/or a coding region and/or non-translated sequences (i.e. introns, 5′- and 3′-untranslated sequences);or

(ii) mRNA or cDNA corresponding to the coding regions (i.e. exons) and 5′- and 3′-untranslated sequences of the gene.

The term “gene” is also used to describe synthetic or fusion molecules encoding all or part of a functional product. Preferred seed formation genes of the present invention may be derived from a naturally-occurring seed formation gene by standard recombinant techniques. Generally, an seed formation gene may be subjected to mutagenesis to produce single or multiple nucleotide substitutions, deletions and/or additions.

Nucleotide insertional derivatives include 5′ and 3′ terminal fusions as well as intra-sequence insertions of single or multiple nucleotides. Insertional nucleotide sequence variants are those in which one or more nucleotides are introduced into a predetermined site in the nucleotide sequence although random insertion is also possible with suitable screening of the resulting product.

Deletional variants are characterised by the removal of one or more nucleotides from the sequence.

Substitutional nucleotide variants are those in which at least one nucleotide in the sequence has been removed and a different nucleotide inserted in its place. Such a substitution may be “silent” in that the substitution does not change the amino acid defined by the codon. Alternatively, substituents are designed to alter one amino acid for another similar acting amino acid, or amino acid of like charge, polarity, or hydrophobicity

As used herein, the term “FIS gene” and variants such as “FIS1 gene”, “FIS2 gene” and “FIS3 gene” shall be taken to refer to a wild-type or functional gene as hereinbefore defined which encodes a functional FIS polypeptide at a biologically-effective level. Consistent with nomenclature known to those skilled in the art, a FIS1 polypeptide is encoded by a FIS1 gene, a FIS2 polypeptide is encoded by a FIS2 gene and a FIS3 polypeptide is encoded by a FIS3 gene.

Preferred FIS genes, the expression of which is intended to be modified by the performance of the invention, include the FIS1, FIS2 and FIS3 genes exemplified herein and homologues, analogues and derivatives thereof.

For the purposes of nomenclature, the FIS1 gene comprises a sequence of nucleotides which is at least about 50% identical to the nucleotide sequence set forth in SEQ ID NO:4 or SEQ ID NO:5. The nucleotide sequence set forth in SEQ ID NO:4 relates to the FIS1 cDNA and the nucleotide sequence set forth in SEQ ID NO:5 relates to the FIS1 genomic gene sequence.

For the purposes of nomenclature, the FIS2 gene comprises a sequence of nucleotides which is at least about 50% identical to the nucleotide sequence set forth in SEQ ID NO:6 or SEQ ID NO:7. The nucleotide sequence set forth in SEQ ID NO:6 relates to the FIS2 cDNA and the nucleotide sequence set forth in SEQ ID NO:7 relates to the FIS2 genomic gene sequence.

For the purposes of nomenclature, the FIS3 gene comprises a sequence of nucleotides which is at least about 50% identical to the nucleotide sequence set forth in SEQ ID NO:8 or SEQ ID NO:9. The nucleotide sequence set forth in SEQ ID NO:8 relates to the FIS3 cDNA and the nucleotide sequence set forth in SEQ ID NO:9 relates to the FIS3 genomic gene sequence.

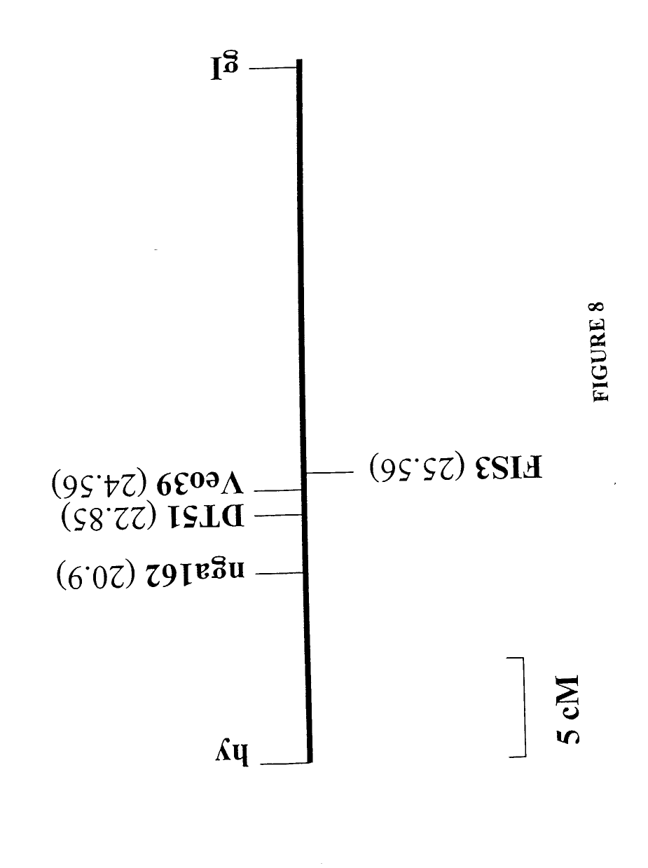

The FIS3 gene comprises either the nucleotide sequence set forth in SEQ ID NO:8 or SEQ ID NO:9, or a complementary sequence thereto, or a sequence of nucleotides which is at least capable of hybridizing under at least low stringency conditions to that region of chromosome 3 of Arabidopsis thaliana which maps between the markers m317 and DWF1 as set forth in FIG. 8B and which encode a FIS3 polypeptide which is capable of modulating autonomous seed development and/or partial autonomous endosperm development and/or autonomous embryogenesis in a plant.

TABLE 1

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpcn |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nehex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl) | Nbhm |
| | | glycine | |
| D-N-methylcysteine | Dnmcys | N-(3,3-diphenylpropyl) | Nbhe |
| | | glycine | |
| D-N-methylglutamine | Dnmgln | N-(3-guanidinopropyl) | Narg |
| | | glycine | |
| D-N-methylglutamate | Dmnglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl)) | Nhis |
| | | glycine | |
| D-N-methylleucine | Dnmleu | N-(3-indolylyethyl) | Nhtrp |
| | | glycine | |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo | Mhphe |
| | | phenylalanine | |
| L-α-methylisoleucine | Mile | N-(2-methylthioethyl) | Nmet |
| | | glycine | |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomo | Nmhphe |
| | | phenylalanine | |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

As used herein, the term “fis gene” shall be taken to refer to a mutant or biologically-ineffective allele of a FIS gene as hereinbefore defined.

By “biologically-ineffective” is meant that a stated integer is not capable of performing its normal biological role in the cell with respect to autonomous seed development and/or partial autonomous endosperm development and/or autonomous embryogenesis.

Particularly preferred chemical mutagens include EMS and methanesulfonic acid ethyl ester. As will be known to those skilled in the art, EMS generally introduces point mutations into the genome of a cell in a random non-targeted manner, such that the number of point mutations introduced into any one genome is proportional to the concentration of the mutagen used. Accordingly, in order to identify a particular mutation, large populations of seed are generally treated with EMS and the effect of the mutation is screened in the M2 seed. Notwithstanding that this is the case, the fis2 and fis3 mutant alleles described herein were identified in EMS-mutagenised lines of Arabidopsis thaliana. Methods for the application and use of chemical mutagens such as EMS are well-known to those skilled in the art.

Preferred irradiation means include ultraviolet and gamma irradiation of whole plants, plant parts and/or seed to introduce point mutations into one or more of the FIS genes present in the genome thereof or alternatively, to create chromosomal deletions in the region of said FIS genes. Methods for the application and use of such mutagens are well-known to those skilled in the art.

Insertional inactivation by molecular means may be achieved by introducing a DNA molecule into one or more of the FIS genes present in the genome of a plant such that the regulatory region and/or reading frame of the FIS gene is disrupted, thereby resulting in either no FIS polypeptide being expressed or a mutant fis polypeptide (i.e. a truncated or biologically ineffective polypeptide) being expressed in the maternally-derived cells, tissues or organs of the plant. Alternatively, a nucleic acid molecule which is capable of insertionally-inactivating a FIS gene may not be inserted directly into the regulatory region or structural regions of said gene, but in the chromatin which is adjacent thereto, such that the insertion promotes a change in chromatin structure which prevents or inhibits expression of the FIS gene or at least reduces expression of the FIS gene to a biologically-ineffective level in the maternally-derived cells, tissues or organs of the plant.

Preferred DNA molecules for insertional inactivation of a FIS gene include gene targeting molecules, transposon molecules, T-DNA molecules and other nucleic acid molecules which comprise one or more translation stop codons or are capable of altering the reading frame of a FIS gene when inserted therein or alternatively, are capable of disrupting one or more regulatory regions essential for expression of a FIS gene in the maternal cells, tissues or organs of the plant. The use of gene targeting molecules, transposon molecules, T-DNA molecules and nucleic acid molecules which comprise one or more translation stop codons is particularly preferred as such molecules may be introduced at any appropriate site within the open reading frame of a FIS gene to prevent the expression of a biologically effective FIS polypeptide.

As used herein, a “gene-targeting molecule” is an isolated nucleic acid molecule which is capable of being introduced into a target genetic sequence within the genome of a plant by homologous recombination, wherein said nucleic acid molecule comprises one or more nucleotide sequences to facilitate said homologous recombination linked to additional nucleotide sequences which are non-homologous to the target genetic sequence, such that the nucleotide sequence of the target genetic sequence is altered following insertion of the gene-targeting molecule. In the present context, a gene-targeting molecule will preferably comprise nucleotide sequences capable of disrupting the open reading frame of a FIS gene when inserted into the homologous region thereof, flanked by one or more nucleotide sequences which are homologous to said FIS gene to facilitate insertion of the gene-targeting molecule into said FIS gene by means of homologous recombination.

Additional means for inhibiting, interrupting or otherwise reducing the expression of a FIS polypeptide include means which target transcription and/or mRNA stability and/or mRNA turnover and/or accessibility of mRNA to ribosomes or polysomes. Such means include the use of antisense molecules, ribozyme molecules, gene silencing molecules and the like introduced into the cell in an expressible format and expressed therein.

In the context of the present invention, an antisense molecule is an RNA molecule which is transcribed from the complementary strand of a nuclear FIS gene to that which is normally transcribed to produce a “sense” mRNA molecule capable of being translated into a FIS polypeptide. The antisense molecule is therefore complementary to the sense mRNA, or a part thereof. Although not limiting the mode of action of the antisense molecules of the present invention to any specific mechanism, the antisense RNA molecule possesses the capacity to form a double-stranded mRNA by base pairing with the FIS-encoding sense mRNA, which may prevent translation of the sense mRNA and subsequent synthesis of a FIS polypeptide product.

Ribozymes are synthetic RNA molecules which comprise a hybridising region complementary to two regions, each of at least 5 contiguous nucleotide bases in the target sense mRNA. In addition, ribozymes possess highly specific endoribonuclease activity, which autocatalytically cleaves the target sense mRNA. A complete description of the function of ribozymes is presented by Haseloff and Gerlach (1988) and contained in International Patent Application No. WO89/05852. The present invention extends to ribozymes which target a sense mRNA encoding a polypeptide involved in seed formation, such as the fis2 polypeptide described herein, thereby hybridising to said sense mRNA and cleaving it, such that it is no longer capable of being translated to synthesise a functional polypeptide product.

In the context of the present invention, gene silencing molecules are molecules which comprise nucleotide sequences complementary to the nucleotide sequence of an antisense mRNA which is complementary to a FIS sense mRNA encoding a FIS polypeptide, linked in head-to-head or tail-to-tail configuration to a part or region of said sense mRNA such that the gene silencing molecule is capable of being transcribed into mRNA which has self-complementarity. Whilst not being bound by any theory or mode of action, a gene silencing molecule has the potential to form a secondary structure such as a hairpin loop in the nucleus and/or cytosol of a cell and to sequester sense mRNA which is transcribed therein, such that single-stranded regions of the sequestered mRNA are rapidly degraded and/or a translationally-inactive complex is formed.

According to this embodiment, the present invention provides a ribozyme, antisense or gene silencing molecule comprising a sequence of contiguous nucleotide bases which are able to form a hydrogen-bonded complex with a sense mRNA encoding a fis polypeptide described herein, to reduce translation of said mRNA. Although the preferred antisense and/or ribozyme and/or gene silencing molecules hybridise to at least about 10 to 20 nucleotides of the target molecule, the present invention extends to molecules capable of hybridising to at least about 50-100 nucleotide bases in length, or a molecule capable of hybridising to a full-length or substantially full-length mRNA.

In yet a further embodiment of the invention, expression of a FIS polypeptide may be inhibited, interrupted or otherwise reduced by introducing to the cell a sense molecule, for example a co-suppression molecule or dominant-negative sense molecule in an expressible format and expressing said molecule therein.

The term “sense molecule” as used herein shall be taken to refer to an isolated nucleic acid molecule which encodes or is complementary to an isolated nucleic acid molecule which encodes a FIS polypeptide involved in autonomous seed development, in particular a FIS1, FIS2 or FIS3 polypeptide or a homologue, analogue or derivative thereof, wherein said nucleic acid molecule is provided in a format suitable for its expression to produce a recombinant polypeptide when said sense molecule is introduced into a host cell by transfection or transformation.

A “co-suppression molecule” is a sense molecule which is capable of producing co-suppression when introduced and optionally, expressed in a cell.

Co-suppression is the reduction in expression of an endogenous gene that occurs when one or more copies of said gene, or one or more copies of a substantially similar gene are introduced into the cell. The present invention clearly extends to the use of co-suppression to inhibit the expression of a FIS gene as described herein.

In the present context, the term “dominant-negative sense molecule” shall be taken to mean a sense molecule as defined herein which comprises a nucleotide sequence which encodes a polypeptide which is capable of inhibiting, preventing or reducing the biological action of a FIS polypeptide, thereby enhancing or facilitating autonomous seed development and/or autonomous endosperm development and/or autonomous embryogenesis.

As will be known to those skilled in the art, a dominant negative sense molecule derived from a FIS polypeptide of the invention will lack the biological activity of the full-length FIS polypeptide.

Preferred dominant-negative sense molecules of the invention will comprise at least one or more functional protein domains of the wild-type FIS protein. For example, a dominant-negative sense molecule which is capable of reducing expression of the FIS1 polypeptide may comprise only an acidic region and/or putative receptor binding domain (e.g. TNFR/NGFR domain or RGD tripeptide, etc.) such that it is capable of competing with a biologically-active FIS1 polypeptide for binding to another protein or receptor, thereby inhibiting the effect of said biologically-active FIS1 polypeptide. Similarly, a dominant-negative sense molecule which is capable of reducing expression of the FIS1 polypeptide may comprise a zinc-finger domain of the FIS2 polypeptide as described herein, such that it is capable of competing with the biologically-active FIS2 polypeptide for binding. The present invention clearly extends to the use of isolated nucleotide sequences encoding any and all combinations of the protein domains which are present in the FIS poypeptides described herein for the purpose of producing such dominant-negative sense molecules.

It is understood in the art that certain modifications, including nucleotide substitutions amongst others, may be made to the dominant-negative sense molecule, co-suppression molecule, gene-targeting molecule, transposon molecule, T-DNA molecule, antisense, ribozyme or gene-silencing molecule of the present invention, without destroying the efficacy of said molecules in inhibiting the expression of the FIS gene. It is therefore within the scope of the present invention to include any nucleotide sequence variants, homologues, analogues, or fragments of the said gene encoding same. However, in the case of gene-silencing molecules, ribozymes and antisense molecules, those skilled in the art will be aware that it is necessary for such nucleotide sequence variants to be capable of hybridising to the biologically active FIS gene sequence or to sense mRNA encoded therefor.

A dominant-negative sense molecule or an antisense molecule or a ribozyme molecule or a gene-targeting molecule or transposon molecule or T-DNA molecule or a co-suppression molecule or gene-silencing molecule capable of targeting expression of a FIS gene in a plant will preferably comprise a nucleotide sequence having at least about 60-70% identity, more preferably at least about 70-80% identity, still more preferably at least about 80-90% identity or a treat about 95-99% identity to the nucleotide sequence of a FIS1 or FIS2 gene set forth in any one of SEQ ID NO:4, SEQ ID NO:5, SEQ ID NO:6, SEQ ID NO:7, SEQ ID NO:8 or SEQ ID NO:9 or a complementary nucleotide sequence thereto. In an alternative embodiment, a dominant-negative sense molecule or an antisense molecule or a ribozyme molecule or a gene-targeting molecule or transposon molecule or T-DNA molecule, or a co-suppression molecule or gene-silencing molecule capable of targeting expression of a FIS gene in a plant will preferably comprise a nucleotide sequence which is capable of hybridizing under at least low stringency conditions, more preferably under at least moderate stringency conditions and even more preferably under at least high stringency conditions, to any one of SEQ ID NO:4, SEQ ID NO:5, SEQ ID NO:6, SEQ ID NO:7, SEQ ID NO:8 or SEQ ID NO:9 or to that region of chromosome 3 of Arabidopsis thaliana which maps between the markers m317 and DWF1 as set forth in FIG. 9B and which encode a FIS3 polypeptide which is capable of modulating autonomous seed development and/or partial autonomous endosperm development and/or autonomous embryogenesis in a plant.

In a further alternative embodiment, the dominant-negative sense molecule or an antisense molecule or a ribozyme molecule or a gene-targeting molecule or a co-suppression molecule is derived from the genomic equivalent of the Arabidopsis thaliana FIS1, FIS2 or FIS3 gene exemplified herein.

The present invention further extends to the mutation or insertional inactivation of such genomic equivalents in order to produce crop and horticultural plants capable of autonomous endosperm development and/or autonomous embryogenesis and/or autonomous seed development and/or apomictic development.

By “genomic equivalent” is meant a homologue of a FIS gene which is derived from another plant species. Such genomic equivalents may be isolated without undue experimentation, using any of the methods known to those skilled in the art, for example by hybridization, PCR, expression screening using antibodies or by functional assays.

Preferred genomic equivalents of the Arabidopsis thaliana FIS genes described herein are derived from crop plants which produce fruit having seed, especially crop plants which produce fruits having large numbers of seed or stone fruit.

More preferably, the genomic equivalents of the Arabidopsis thaliana FIS genes are derived from mango, pawpaw, olives, apple, cherry, plum, peach, apricot, grape, passionfruit, date, fig, tomato, pear, tamarillo, quince, strawberry, blackberry, gooseberry, loganberry, Capsicum spp. and citrus plants, amongst others.

As will be known to those skilled in the art, the efficacy of a dominant-negative sense molecule or an antisense molecule or a ribozyme molecule or a gene-targeting molecule or transposon molecule or T-DNA molecule or a co-suppression molecule or gene-silencing molecule is dependent upon it being introduced and preferably, expressed in the maternal cell, tissue or organ or a progenitor cell, tissue or organ thereof. Such introduction and expression may be facilitated by presenting said dominant-negative sense molecule or an antisense molecule or a ribozyme molecule or a gene-targeting molecule or transposon molecule or T-DNA molecule or a co-suppression molecule or gene-silencing molecule in a genetic construct.

The present invention clearly extends to the use of genetic constructs designed to facilitate the introduction and/or expression of a dominant negative sense molecule, antisense molecule, ribozyme molecule, co-suppression molecule or gene-targeting molecule or transposon molecule or T-DNA molecule or gene-silencing molecule in a plant cell and preferably in a maternal cell, tissue or organ or a progenitor cell, tissue or organ thereof.

Those skilled in the art will also be aware that expression of a dominant-negative sense, antisense, ribozyme, gene-targeting, co-suppression or gene-silencing molecule may require said molecule to be placed in operable connection with a promoter sequence. The choice of promoter for the present purpose may vary depending upon the level of expression required and/or the tissue, organ and species in which expression is to occur.

Reference herein to a “promoter” is to be taken in its broadest context and includes the transcriptional regulatory sequences of a classical eukaryotic genomic gene, including the TATA box which is required for accurate transcription initiation, with or without a CCAAT box sequence and additional regulatory elements (i.e. upstream activating sequences, enhancers and silencers) which alter gene expression in response to developmental and/or external stimuli, or in a tissue-specific manner. In the context of the present invention, the term “promoter” also includes the transcriptional regulatory sequences of a classical prokaryotic gene, in which case it may include a −35 box sequence and/or a −10 box transcriptional regulatory sequences.

In the present context, the term “promoter” is also used to describe a synthetic or fusion molecule, or derivative which confers, activates or enhances expression of said sense molecule in a cell. Preferred promoters may contain additional copies of one or more specific regulatory elements, to further enhance expression and/or to alter the spatial expression and/or temporal expression of a nucleic acid molecule to which it is operably connected. For example, copper-responsive regulatory elements may be placed adjacent to a heterologous promoter sequence driving expression of a nucleic acid molecule to confer copper inducible expression thereon.

Placing a nucleic acid molecule under the regulatory control of a promoter sequence means positioning said molecule such that expression is controlled by the promoter sequence. A promoter is usually, but not necessarily, positioned upstream or 5′ of a nucleic acid molecule which it regulates. Furthermore, the regulatory elements comprising a promoter are usually positioned within 2 kb of the start site of transcription of a sense, antisense, ribozyme, gene-targeting molecule or co-suppression molecule or chimeric gene comprising same. In the construction of heterologous promoter/structural gene combinations it is generally preferred to position the promoter at a distance from the gene transcription start site that is approximately the same as the distance between that promoter and the gene it controls in its natural setting, i.e., the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

Examples of promoters suitable for use in genetic constructs of the present invention include promoters derived from the genes of viruses, yeasts, molds, bacteria, insects, birds, mammals and plants which are capable of functioning in isolated plant cells, preferably in the maternally-derived cells of a plant or the cells, tissues and organs derived therefrom. The promoter may regulate the expression of the sense, antisense, ribozyme, gene-targeting molecule, co-suppression or gene-silencing molecule constitutively, or differentially with respect to the tissue in which expression occurs or, with respect to the developmental stage at which expression occurs, or in response to external stimuli such as physiological stresses, pathogens, or metal ions, amongst others.

Promoters suitable for use according to this embodiment are further capable of functioning in cells derived from both monocotyledonous and dicotyledonous plants, including broad acre crop plants or horticultural crop plants.

Examples of promoters useful in performing this embodiment include the CaMV 35S promoter, NOS promoter, octopine synthase (OCS) promoter, Arabidopsis thaliana SSU gene promoter, the meristem-specific promoter (meri1),napin seed-specific promoter, and the like. In addition to the specific promoters identified herein, cellular promoters for so-called housekeeping genes are useful.

In a particularly preferred embodiment, the promoter may be derived from a genomic clone comprising a seed formation gene, in particular derived from the genomic gene equivalents of the A. thaliana FIS1, FIS2 OR FIS3 gene referred to herein.

The genetic construct may further comprise a terminator sequence and be introduced into a suitable host cell where it is capable of being expressed to produce a recombinant dominant-negative polypeptide gene product or alternatively, a co-suppression molecule, a ribozyme, gene silencing or antisense molecule.

The term “terminator” refers to a DNA sequence at the end of a transcriptional unit which signals termination of transcription. Terminators are 3′-non-translated DNA sequences containing a polyadenylation signal, which facilitates the addition of polyadenylate sequences to the 3′-end of a primary transcript. Terminators active in cells derived from viruses, yeasts, moulds, bacteria, insects, birds, mammals and plants are known and described in the literature. They may be isolated from bacteria, fungi, viruses, animals and/or plants.

Examples of terminators particularly suitable for use in the genetic constructs of the present invention include the nopaline synthase (NOS) gene terminator of Agrobacterium tumefaciens, the terminator of the Cauliflower mosaic virus (CaMV) 35S gene, the zein gene terminator from Zea mays, the Rubisco small subunit (SSU) gene terminator sequences and subclover stunt virus (SCSV) gene sequence terminators, amongst others.

Those skilled in the art will be aware of additional promoter sequences and terminator sequences which may be suitable for use in performing the invention. Such sequences may readily be used without any undue experimentation.

The genetic constructs of the invention may further include an origin of replication sequence which is required for replication in a specific cell type, for example a bacterial cell, when said genetic construct is required to be maintained as an episomal genetic element (eg. plasmid or cosmid molecule) in said cell.

Preferred origins of replication include, but are not limited to, the f1-ori and co/E1 origins of replication.

The genetic construct may further comprise a selectable marker gene or genes that are functional in a cell into which said genetic construct is introduced.

As used herein, the term “selectable marker gene” includes any gene which confers a phenotype on a cell in which it is expressed to facilitate the identification and/or selection of cells which are transfected or transformed with a genetic construct of the invention or a derivative thereof.

Suitable selectable marker genes contemplated herein include the ampicillin resistance (Amp^{r}), tetracycline resistance gene (Tc^{r}), bacterial kanamycin resistance gene (Kan^{r}), phosphinothricin resistance gene, neomycin phosphotransferase gene (nptII), hygromycin resistance gene, β-glucuronidase (GUS) gene, chloramphenicol acetyltransferase (CAT) gene and luciferase gene, amongst others.

In a preferred embodiment, the subject method comprises the additional first step of transforming the cell, tissue, organ or organism with a nucleic acid molecule which comprises the sense, antisense, ribozyme, co-suppression or gene-targeting molecule or transposon or T-DNA molecule. As discussed supra this nucleic acid molecule may be contained within a genetic construct. The nucleic acid molecule or a genetic construct comprising same may be introduced into a cell using any known method for the transfection or transformation of said cell. Wherein a cell is transformed by the genetic construct of the invention, a whole organism may be regenerated from a single transformed cell, using any method known to those skilled in the art.

By “transfect” is meant that the introduced nucleic acid molecule is introduced into said cell without integration into the cell's genome.

By “transform” is meant that the introduced nucleic acid molecule or genetic construct comprising same or a fragment thereof comprising a FIS gene sequence is stably integrated into the genome of the cell.

Means for introducing recombinant DNA into plant tissue or cells include, but are not limited to, transformation using CaCl₂ and variations thereof, in particular the method described by Hanahan (1983), direct DNA uptake into protoplasts (Krens et al, 1982; Paszkowski et al, 1984), PEG-mediated uptake to protoplasts (Armstrong et al, 1990) microparticle bombardment, electroporation (Fromm et al., 1985), microinjection of DNA (Crossway et al., 1986), microparticle bombardment of tissue explants or cells (Christou et al, 1988; Sanford, 1988), vacuum-infiltration of tissue with nucleic acid, or in the case of plants, T-DNA-mediated transfer from Agrobacterium to the plant tissue as described essentially by An et al.(1985), Herrera-Estrella et al. (1983a, 1983b, 1985).

For microparticle bombardment of cells, a microparticle is propelled into a cell to produce a transformed cell. Any suitable biolistic cell transformation methodology and apparatus can be used in performing the present invention. Exemplary apparatus and procedures are disclosed by Stomp et al. (U.S. Pat. No. 5,122,466) and Sanford and Wolf (U.S. Pat. No. 4,945,050). When using biolistic transformation procedures, the genetic construct may incorporate a plasmid capable of replicating in the cell to be transformed.

Examples of microparticles suitable for use in such systems include 1 to 5 μm gold spheres. The DNA construct may be deposited on the microparticle by any suitable technique, such as by precipitation.

Alternatively, wherein the cell is derived from a multicellular organism and where relevant technology is available, a whole organism may be regenerated from the transformed cell, in accordance with procedures well known in the art.

Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The term “organogenesis”, as used herein, means a process by which shoots and roots are developed sequentially from meristematic centres.

The term “embryogenesis”, as used herein, means a process by which shoots and roots develop together in a concerted fashion (not sequentially), whether from somatic cells or gametes.

The regenerated transformed plants may be propagated by a variety of means, such as by clonal propagation or classical breeding techniques. For example, a first generation (or T1) transformed plant may be selfed or crossed to another T1 plant and homozygous second generation (or T2) transformants selected.

In the case of woody fruit crops such as citrus and grapes which are highly heterozygous and propagated vegetatively from cuttings, the genes to be introduced must be dominant in action and the cultivar identity must be maintained by using the primary transformants directly, for example by generating clonal derivatives of primary transformants.

It is preferred in the commercial application of the invention to the production of soft-seeded fruits that transgenic plants having reduced expression of FIS (i.e. knock-out plants) are further made male-sterile by any means known to those skilled in the art, preferably by the expression of a gene construct which induces male-sterility in plants as a dominant phenotype, such as by the expression of a barnase gene or a gene encoding a cytotoxin under control of an anther-specific or tapetum-specific gene promoter. Where the barnase gene or a gene encoding a cytotoxin is used to induce male-sterility, this should only need to be present in the heterozygous state to observe the male-sterile phenotype. In this way, there is no initiation of seed formation from those cells of the primary transformant which do not contain or express the introduced gene. This strategy is particularly relevant to the application of the invention in cases where fruits comprise multiple seeds, such as citrus fruits, grapes, berries, pears, apples and tomato, amongst others. In the case of stone fruit, although some fruit having normal seed may initiate in the absence of male-sterility, it may be possible to screen and select for those fruit having soft seed.

In applications of the invention to the production of apomictic plants by an autonomous seed development mechanism (as opposed to a pseudogamous mechanism which requires pollination to initiate seed development), it is also preferred that plants are made male-sterile to reduce or prevent any “leakiness” in the downregulation of endogenous FIS gene expression, thereby ensuring that all seed which are produced by transgenic plants are the●products of apomixis and not hybrid seed.

In the case of woody plants such as citrus and grapes which are generated by cuttings, it is particularly preferred to employ a strategy wherein dominant-acting male-sterility-inducing gene constructs and the gene construct capable of down-regulating expression of the negative regulator of seed formation are introduced into plant material and primary transformants selected which contain both genes integrated into their genome. As with all transformation strategies, a large number of primary transformants should be generated to facilitate elimination of those transformants wherein the introduced gene constructs are inserted into housekeeping genes or otherwise have an adverse effect on the plant, including an adverse effect on the quality or yield of the plant products derived therefrom. Primary transformants are propagated by cuttings to generate lines of transgenic plant material which either contain single or multiple copies of the introduced gene construct(s) and the mature plants derived therefrom assayed for product quality.

Plants may be made male-sterile before or after the gene construct targeting fis gene expression is introduced into plants or alternatively, at the same time as the gene construct targeting fis gene expression is introduced into plants. Wherein the plants are made male-sterile before or after introducing the gene construct targeting FIS gene expression, this is best achieved by making such plants homozygous for one or both of the introduced genes (i.e. the male-sterility gene and/or the gene construct targeting FIS gene expression). Persons skilled in the art will be aware of the most preferred means for making plants homozygous for one or both of the introduced genes for any particular plant species-of-interest. Clearly, in the case of vegetatively-propagated species, such an approach is not viable.

Preferably, plants are made male-sterile at the same time as the gene construct targeting fis gene expression is introduced into plants. Such an approach is particularly preferred in the case of woody plants which are propagated vegetatively. In such cases it is even more preferable to include the male-sterility-inducing gene on the same vector as the gene construct which downregulates FIS gene expression in the plant. Those skilled in the art will also be aware of the advantage of having the male-sterile phenotype cosegregate with the introduced gene construct which targets fis gene expression. This advantage may be derived advantageously by having both gene [symbol] cassettes [symbol] located on the same gene construct such that they are closely linked, to prevent recombination therebetween occurring at a high frequency, in the primary transformants and in the progeny plants derived therefrom

Methods for the production of male-sterile plants will be known to those skilled in the art and the present invention is not limited by such means.

The regenerated transformed organisms contemplated herein may take a variety of forms. For example, they may be chimeras of transformed cells and non-transformed cells; clonal transformants (e.g., all cells transformed to contain the expression cassette); grafts of transformed and untransformed tissues (e.g., in plants, a transformed root stock grafted to an untransformed scion).

The above-mentioned dominant-negative sense molecules, antisense molecules, ribozyme molecules, gene-targeting molecules, transposons, T-DNA molecules, gene silencing molecules and co-suppression molecules are particularly useful for reducing or eliminating the expression of particular FIS genes in plants, to produce plants which at least exhibit autonomous endosperm development.

A transformed plant comprising the introduced nucleic acid molecule contemplated herein to reduce the expression of FIS polypeptide will preferably exhibit a phenotype which is substantially identical to the autonomous seed formation phenotype of the fis1, fis2 or fis3 mutant described herein.

Arrested embryo development which results from inhibition of expression of the FIS gene may be concomitant with autonomous endosperm development in the plant into which the subject dominant-negative sense molecule or an antisense molecule or a ribozyme molecule or a gene-targeting molecule or a co-suppression molecule is introduced and expressed. As exemplified herein, in the absence of FIS2 expression or expression of any of the protein domains of the FIS1 polypeptide referred to herein, Arabidopsis thaliana ecotype Landsberg plants produce autonomous seed or seed-like structures which lack a functional embryo and are softer than wild-type seed.

In fact, the invention is particularly useful to produce parthenocarpic fruit or “seedless fruit” which lacks a fully-developed embryo not normally produced by wild or naturally-occurring organisms belonging to the same genera or species as the genera or species from which the transfected or transformed cell is derived. Such seedless fruit may, in fact, include fruits having soft seed which are present at a level which allows the fruit to be marketed as “less seedy” than wild-type fruit.

Preferred target plants in which the invention may be performed include stone fruits such as apricots and peaches, citrus fruits such as oranges, lemons, grapefruits, mandarins and tangelos, amongst others, in addition to grapes, apples, melons, pears, and berries, amongst others.

Preferably, the inventive method is used to develop plants which autonomously form seed comprising an embryo and an endosperm.

Alternatively or in addition, such plants may be apomictic, in which case they will autonomously develop fully-fertile seed. As the presently described genes have been shown to at least be capable of repressing autonomous embryogenesis and partial autonomous endosperm development in vivo, the application of such genes to the development of fully-fertile apomictic seeds, those skilled in the art will also be aware of the particular utility of the presently-described FIS genes in producing plants which are capable of autonomously forming fully-fertile seed (i.e. apomictic plants).

Preferred target plants in which this embodiment of the invention may be performed include monocotyledonous or dicotyledonous broadacre or horticultural crop plants, are those plants which produce seed of agronomic value, such as grain crop plants, in particular rice, wheat, maize, rape, rye, safflower, sunflower, millet and barley, amongst others.

The present inventors are aware of the possible existence of one or more modifier genes which, in combination with the dominant-negative sense molecule, antisense molecule, ribozyme molecule, gene-targeting molecule, transposon, T-DNA molecule, gene-silencing molecule or co-suppression molecule which comprise the FIS gene sequences described herein, interact to produce plants capable of complete autonomous embryogenesis in addition to complete autonomous endosperm development, wherein the mature seed are fully-fertile. It is clearly within the scope of the present invention to include the optional use of nucleotide sequences derived from the presently-described FIS genes in combination with any other gene(s) or alternatively, any sense molecule, dominant-negative sense molecule, antisense molecule, ribozyme molecule, gene-targeting molecule, transposon, T-DNA molecule, gene-silencing molecule or co-suppression molecule comprising said other gene(s), to perform the inventive method.

As an alternative to the introduction of specific modifier genes in combination with the dominant-negative sense molecule, antisense molecule, ribozyme molecule, gene-targeting molecule, transposon, T-DNA molecule, gene-silencing molecule or co-suppression molecule of the invention, it is also within the capabilities of the skilled artisan to introduce a dominant-negative sense molecule, antisense molecule, ribozyme molecule, gene-targeting molecule, transposon, T-DNA molecule, gene-silencing molecule or co-suppression molecule into a genetic background which expresses the modifier gene at a level which is such that introduction of said inventive molecules thereto will be sufficient to produce a plant which is capable of autonomous seed development and/or autonomous endosperm development and/or autonomous embryogenesis and preferably, an apomictic plant.

A second aspect of the invention clearly extends to the isolated nucleic acid molecules which are used to inhibit, prevent or interrupt the expression of a FIS polypeptide in a plant according to the inventive method, including those genomic equivalents of the Arabidopsis thaliana FIS polypeptides exemplified herein.

Preferably, the nucleic acid molecule according to this aspect of the invention will comprise a dominant-negative sense molecule or an antisense molecule or a ribozyme molecule or a gene-targeting molecule or a co-suppression molecule or a gene silencing molecule which comprises a nucleotide sequence which is derived from a FIS gene as described herein or a genomic equivalent thereof.

A third aspect of the invention clearly extends to a transgenic plant or a plant cell, tissue, organ produced according to the method described herein, including the seed produced by said plant and progeny plants derived therefrom which are capable of reproducing by apomictic means.

According to this aspect, the invention provides a cell which has been transformed or transfected with the subject nucleic acid molecule or a dominant-negative sense molecule or an antisense molecule or a ribozyme molecule or a gene-targeting molecule or a co-suppression molecule which is derived from a FIS gene, preferably in an expressible form.

A further aspect of the invention provides an isolated nucleic acid molecule comprising a nucleotide sequence which encodes or is complementary to a nucleotide sequence which encodes a polypeptide, protein or enzyme which is capable of regulating autonomous endosperm development in a plant.

Preferably, the polypeptide, protein or enzyme is further capable of regulating autonomous embryogenesis and more preferably, autonomous seed development in a plant.

By “capable of regulating endosperm development” means that the polypeptide, protein or enzyme is involved in asexual seed development in plants at least to the extent that a disruption of expression or reduction in the level of expression of said polypeptide, protein or enzyme in the plant induces at least partial autonomous endosperm development therein.

By “capable of regulating embryogenesis” means that the polypeptide, protein or enzyme is involved in asexual seed development in plants at least to the extent that a disruption of expression or reduction in the level of expression of said polypeptide, protein or enzyme in the plant induces at least partial autonomous embryogenesis therein.

By “capable of regulating seed development” means that the polypeptide, protein or enzyme is involved in asexual seed development in plants at least to the extent that a disruption of expression or reduction in the level of expression of said polypeptide, protein or enzyme in the plant induces at least partial autonomous endosperm development and partial autonomous embryogenesis therein and preferably induces the autonomous development of fully-fertile seeds.

In one alternative embodiment, the nucleic acid molecule of the invention encodes or is complementary to a nucleic acid molecule which encodes a FIS polypeptide, protein or enzyme or a protein domain thereof according to any one or more embodiments described herein or a genomic equivalent thereof.

Alternatively or in addition, the isolated nucleic acid molecule of the invention comprises a FIS gene which is involved in fertilization-independent seed production in a plant.

In the context of the present invention, “fertilization-independent seed production” means the autonomous formation of fertile seed or seed-like structures comprising an embryo and/or endosperm with or without a seed coat, from any of the organs forming the gynoecium or contained within the gynoecium. More particularly, fertilization-independent seed production results in the autonomous formation of fertile seed or seed-like structures from the megaspore and/or non-archesporial cells such as those forming the nucellus or integument.

Accordingly, the present invention clearly encompasses those isolated genes which are expressed to regulate autonomous seed formation in any plant species, regardless of whether or not that gene is capable of resulting in the formation of fully-fertile seed or seed-like structures. Those skilled in the art will recognize that the isolated gene described herein does however perform a critical role in autonomous seed production in plants. The inventors have characterised the FIS (Fertilization Independent Seed) family of genes, at least three genes of which are exemplified herein, designated FIS1, FIS2 and FIS3 and which encode different polypeptide repressors capable of inhibiting autonomous embryogenesis and partial autonomous endosperm development in plants.

Those skilled in the art may readily assay for FIS gene activity of an isolated nucleic acid molecule by determining the ability of an inhibitor of the expression of said nucleic acid molecule, such as a mutagen, an antisense molecule, dominant-negative sense molecule, ribozyme molecule, co-suppression molecule, transposon, T-DNA, gene silencing molecule or gene-targeting molecule as described herein, to induce autonomous endosperm development and/or autonomous embryogenesis and/or autonomous seed formation in a plant.

Alternatively, the activity of the polypeptide encoded by a FIS gene may be inhibited using a ligand which specifically binds thereto, such as an antibody molecule or a peptide, oligopeptide, polypeptide, enzyme or chemical compound which binds to its active site, and the autonomous induction of formation of seed or seed-like structures is assayed. For convenience, the plant being assayed may first be made male-sterile to reduce background self-fertilization events.

Preferably, the isolated nucleic acid molecule of the invention comprises a FIS gene which comprises the sequence of nucleotides set forth in any one of SEQ ID NO:4, SEQ ID NO:5, SEQ ID NO:6, SEQ ID NO:7, SEQ ID NO:8 or SEQ ID NO:9 or a homologue, analogue or derivative thereof or a complementary nucleotide sequence thereto.

For the present purpose, “homologues” of a nucleotide sequence shall be taken to refer to an isolated nucleic acid molecule which is substantially the same as the nucleic acid molecule of the present invention or its complementary nucleotide sequence, notwithstanding the occurrence within said sequence, of one or more nucleotide substitutions, insertions, deletions, or rearrangements.

“Analogues” of a nucleotide sequence set forth herein shall be taken to refer to an isolated nucleic acid molecule which is substantially the same as a nucleic acid molecule of the present invention or its complementary nucleotide sequence, notwithstanding the occurrence of any non-nucleotide constituents not normally present in said isolated nucleic acid molecule, for example carbohydrates, radiochemicals including radionucleotides, reporter molecules such as, but not limited to DIG, alkaline phosphatase or horseradish peroxidase, amongst others.

“Derivatives” of a nucleotide sequence set forth herein shall be taken to refer to any isolated nucleic acid molecule which contains significant sequence identity to said sequence or a part thereof. Generally, the nucleotide sequence of the present invention may be subjected to mutagenesis to produce single or multiple nucleotide substitutions, deletions and/or insertions. Nucleotide insertional derivatives of the nucleotide sequence of the present invention include 5′ and 3′ terminal fusions as well as intra-sequence insertions of single or multiple nucleotides or nucleotide analogues. Insertional nucleotide sequence variants are those in which one or more nucleotides or nucleotide analogues are introduced into a predetermined site in the nucleotide sequence of said sequence, although random insertion is also possible with suitable screening of the resulting product being performed. Deletional variants are characterised by the removal of one or more nucleotides from the nucleotide sequence. Substitutional nucleotide variants are those in which at least one nucleotide in the sequence has been removed and a different nucleotide or nucleotide analogue inserted in its place.

Particularly preferred homologues, analogues or derivatives of the nucleotide sequences set forth in any one of SEQ ID NO:4, SEQ ID NO:5, SEQ ID NO:6, SEQ ID NO:7, SEQ ID NO:8 or SEQ ID NO:9 include any one or more of the isolated nucleic acid molecules selected from the following:

(i) an isolated nucleic acid molecule which comprises a nucleotide sequence which is at least about 60% identical to any one of SEQ ID NO:4, SEQ ID NO:5, SEQ ID NO:6, SEQ ID NO:7, SEQ ID NO:8 or SEQ ID NO:9 or a complementary sequence thereto;

(ii) an isolated nucleic acid molecule which comprises a nucleotide sequence which is at least about 60% identical to at least about 30 contiguous nucleotides of any one of SEQ ID NO:4, SEQ ID NO:5, SEQ ID NO:6, SEQ ID NO:7, SEQ ID NO:8 or SEQ ID NO:9 or a complementary sequence thereto;

(iii) an isolated nucleic acid molecule which is capable of hybridising under at least low stringency conditions to at least about 25-30 contiguous nucleotides of any one of SEQ ID NO:4, SEQ ID NO:5, SEQ ID NO:6, SEQ ID NO:7, SEQ ID NO:8 or SEQ ID NO:9 or a complementary sequence thereto; and

(iv) an isolated nucleic acid molecule which is capable of hybridising under at least low stringency conditions to at least about 25-30 contiguous nucleotides of the RFLP marker designated ve039 or the YAC clone CC7E1 or the p1clones MCB22 or MNH5 or a complementary sequence thereto;

Such homologues, analogues and derivatives may be obtained by any standard procedure known to those skilled in the art, such as by nucleic acid hybridization (Ausubel et al, 1987), polymerase chain reaction (McPherson et al, 1991) screening of expression libraries using antibody probes (Huynh et al, 1985) or by functional assay as exemplified herein.

In nucleic acid hybridizations, genomic DNA, mRNA or cDNA or a part of fragment thereof, in isolated form or contained within a suitable cloning vector such as a plasmid or bacteriophage or cosmid molecule, is contacted with a hybridization-effective amount of a nucleic acid probe derived from any one of SEQ ID NO:4, SEQ ID NO:5, SEQ ID NO:6, SEQ ID NO:7, SEQ ID NO:8 or SEQ ID NO:9 or alternatively, from the RFLP marker designated ve039 or the YAC clone CC7E1 or the p1 clones MCB22 or MNH5, for a time and under conditions sufficient for hybridization to occur and the hybridized nucleic acid is then detected using a detecting means.

Detection is performed preferably by labelling the probe with a reporter molecule capable of producing an identifiable signal, prior to hybridization. Preferred reporter molecules include radioactively-labeled nucleotide triphosphates and biotinylated molecules.

Preferably, variants of the FIS genes exemplified herein, including genomic equivalents, are isolated by hybridisation under medium or more preferably, under high stringency conditions, to the probe.

In the polymerase chain reaction (PCR), a nucleic acid primer molecule comprising at least about 14 nucleotides in length derived from a FIS gene is hybridized to a nucleic acid template molecule and specific nucleic acid molecule copies of the template are amplified enzymatically as described in McPherson et al, (1991), which is incorporated herein by reference.

In expression screening of cDNA libraries or genomic libraries, protein- or peptide-encoding regions are placed operably under the control of a suitable promoter sequence in the sense orientation, expressed in a prokaryotic cell or eukaryotic cell in which said promoter is operable to produce a peptide or polypeptide, screened with a monoclonal or polyclonal antibody molecule or a derivative thereof against one or more epitopes of a FIS polypeptide and the bound antibody is then detected using a detecting means, essentially as described by Huynh et al (1985) which is incorporated herein by reference. Suitable detecting means according to this embodiment include ¹²⁵I-labelled antibodies or enzyme-labelled antibodies capable of binding to the first-mentioned antibody, amongst others.

The nucleic acid molecule of the invention or a homologue, analogue or derivative thereof may be obtained from any plant species.

A still further aspect of the invention provides an isolated promoter sequence which is capable of conferring expression at least in one or more female reproductive cells, tissues or organs of said plant or a progenitor cell, tissue or organ thereof. Preferably, the promoter is capable of conferring expression in the ovule or a progenitor cell thereof or a derivative cell, tissue or organ thereof.

More preferably, the promoter sequence is isolatable as a DNA fragment which is capable of hybridising under at least low stringency conditions to any one or more of the nucleotide sequences set forth in SEQ ID NO:4, SEQ ID NO:5, SEQ ID NO:6, SEQ ID NO:7, SEQ ID NO:8 or SEQ ID NO:9 or a complementary nucleotide sequence thereto and even more preferably to the 5′-region of any one or more of said nucleotide sequences and still even more preferably to the 5′-untranslated regions of any one of SEQ ID NO:4, SEQ ID NO:5, SEQ ID NO:6, SEQ ID NO:7, SEQ ID NO:8 or SEQ ID NO:9 or a complementary nucleotide sequence thereto.

In a particularly preferred embodiment, the promoter at least comprises a nucleotide sequence which corresponds to nucleotide residues 1 to 3142 of SEQ ID NO:5 or a part thereof; or nucleotide residues 1785 to 3142 of SEQ ID NO:5 or a part thereof; or nucleotide residues 1 to 2851 of SEQ ID NO:7 or a part thereof; or nucleotide residues 1531 to 2851 of SEQ ID NO:7 or a part thereof; or nucleotide residues 1 to 1200 of SEQ ID NO:9 or a part thereof.

Alternatively or in addition, the promoter sequence may further comprise the exon1 and/or intron1 sequence of a FIS gene described herein, in particular a FIS gene as described in SEQ ID NO:5 or SEQ ID NO:7 or SEQ ID NO:9.

The present invention clearly extends to the promoter sequence and/or exon1 and/or intron1 sequences in operably connection with a structural gene region derived from the same or a different genetic sequence, optionally in a genetic construct.

A still further aspect of the present invention provides an isolated or recombinant FIS polypeptide or a homologue, analogue, derivative or epitope thereof.

Particularly preferred derivatives of a FIS polypeptide include those peptides, oligopeptides and polypeptides which comprise at least about 5-10 contiguous amino acids derived from any one of SEQ ID NO:1 or SEQ ID NO:2 or SEQ ID NO:3 or which comprise any one of the protein domains of the FIS1 or FIS2 or FIS3 polypeptides described herein or a fragment thereof comprising at least about 5 amino acids in length.

As used herein, the term [symbol] epitope [symbol] refers to a peptide or derivative of a FIS polypeptide which is at least useful for the preparation of antibody molecules, including recombinant antibodies, polyclonal or monoclonal antibody molecules.

It will be apparent from the description provided herein that a recombinant FIS polypeptide or an epitope thereof may be produced by standard means by expressing a sense molecule which comprises a nucleotide sequence which encodes said polypeptide operably under the control of a suitable promoter sequence in a host cell for a time and under conditions sufficient for translation to occur.

As will be known to those skilled in the art, expression of a sense molecule may be carried out in a prokaryotic cell such as a bacterial cell, for example an Escherichia coli cell. Alternatively, such expression may be performed in a eukaryotic cell such as an insect cell, mammalian cell, plant cell or yeast cell, amongst others. In any case, unless the sense molecule is expressed under the control of a strong universal promoter, it is important to select a promoter sequence which is capable of regulating expression in the cell comprising the sense molecule in an expressible format. Persons skilled in the art will be in a position to select appropriate promoter sequences for expression of the sense molecule without undue experimentation.

Examples of promoters useful in performing this embodiment include the CaMV 35S promoter, NOS promoter, octopine synthase (OCS) promoter, Arabidopsis thaliana SSU gene promoter, napin seed-specific promoter, P₃₂ promoter, BK5-T imm promoter, lac promoter, tac promoter, phage lambda L or R promoters, CMV promoter (U.S. Pat. No. 5,168,062), T7 promoter, lacUV5 promoter, SV40 early promoter (U.S. Pat. No. 5,118,627), SV40 late promoter (U.S. Pat. No. 5,118,627), adenovirus promoter, baculovirus P10 or polyhedrin promoter (U.S. Pat. Nos. 5,243,041, 5,242,687, 5,266,317, 4,745,051 and 5,169,784), and the like. In addition to the specific promoters identified herein, cellular promoters for so-called housekeeping genes are useful.

In a preferred embodiment, the recombinant FIS polypeptide or a homologue, analogue, derivative or epitope thereof is provided in a sequencably-pure format or a substantially pure format.

By “sequencably pure” is meant that the subject polypeptide or a homologue, analogue, derivative or epitope thereof is purified sufficiently to facilitate amino acid sequence determination.

Preferably, said polypeptide or a homologue, analogue, derivative or epitope is at least about 20% pure, more preferably at least about 40% pure, even more preferably at least about 60% pure and even more preferably at least about 80% pure or 95% pure on a weight basis.

It is apparent from the description provided herein that the FIS polypeptides are likely to be involved in a range of biological interactions in the regulation of seed development in plants (see for example, the description in Example 16), in particular protein:protein interactions, such as via the acidic region of the FIS1 polypeptide or the repeat structure of the FIS2 polypeptide, amongst others and/or protein:nucleic acid molecule interactions, such as via one or more of the cysteine-rich regions of the FIS1 polypeptide or the zinc-finger motif of the FIS2 polypeptide, amongst others. Such interactions are well known for their effects in regulating gene expression in both prokaryotic and eukaryotic cells, in addition to being critical for DNA replication and in the case of certain viruses, RNA replication.

As used herein, the term “interaction” shall be taken to refer to a physical association between two or more molecules or “partners”, one of which comprises a FIS polypeptide or a protein domain thereof as described herein or a peptide derivative thereof. The association is involved in one or more cellular processes involved in seed development in plants and preferably occurs at least in the maternal cells, tissues or organs, such as in the process of imprinting.

The “association” may involve the formation of an induced magnetic field or paramagnetic field, covalent bond formation such as a disulfide bridge formation between polypeptide molecules, an ionic interaction such as occur in an ionic lattice, a hydrogen bond or alternatively, a van der Waals interaction such as a dipole-dipole interaction, dipole-induced-dipole interaction, induced-dipole-induced-dipole interaction or a repulsive interaction or any combination of the above forces of attraction.

As used herein, the term “FIS partner” shall be taken to mean any amino acid sequence which is derived from a FIS polypeptide and which is capable of directly interacting with one or more peptides, oligopeptides, polypeptides, proteins, RNA molecules and DNA molecules to confer or regulate autonomous endosperm development and/or autonomous embryogenesis and/or autonomous or pseudogamous seed development in plants.

The present invention clearly extends to those peptides, oligopeptides, polypeptides, proteins, RNA molecules and DNA molecules which interact with a FIS partner.

Preferably, the peptides, oligopeptides, polypeptides, proteins, RNA molecules and DNA molecules which interact with a FIS partner are normally regulated by one or more FIS polypeptides.

By appropriate strategies described herein, the peptides, oligopeptides, polypeptides, proteins, RNA molecules and DNA molecules which interact with a FIS partner and the nucleic acid molecules encoding said interacting peptides, oligopeptides, polypeptides and proteins are isolated.

Conventional one-hybrid, two-hybrid and three-hybrid assays may be used to identify and isolate the peptides, oligopeptides, polypeptides, proteins, RNA molecules and DNA molecules which interact with a FIS partner. Such assays are described in detail by Poutney et al. (1997), Bendixen et al.(1994), Vidal et al. (1996a,b), Yang et al. (1995) and Zhang et al. (1996), which are incorporated herein by way of reference.

In such assays, recombinant cells are produced which are capable of expressing both binding partners. In screening applications, a representative random library is generally produced in a cellular host, such that each cell expresses a different peptide, oligopeptide, polypeptide or protein or RNA molecule or DNA molecule, in addition to expressing the FIS partner. The transformed cells of the library may further contain a nucleotide sequence which comprises or encodes a reporter molecule, the expression of which is capable of being modified by the interaction between the binding partners. The cells are cultured for a time and under conditions sufficient for expression of said second nucleotide sequences encoding the partners to occur and cells wherein expression of said reporter molecule is modified are selected.

Alternatively or in addition, the binding partners are further expressed as a fusion protein with a nuclear targeting motif capable of facilitating targeting of said peptide to the nucleus of said host cell where transcription occurs, in particular the yeast-operable SV40 nuclear localisation signal.

The FIS partner and/or its cognate binding partner may also be expressed constitutively on the surface of a bacteriophage, such as by phage display, a process well-known in the art.

In the case of nucleic acid molecule binding partners which interact with the FIS partner, it is preferred that the nucleotide sequences of the random library are placed in operable connection with a nucleic acid molecule which encodes the reporter molecule. Wherein the FIS partner inhibits activity of the other binding partner in vitro, expression of the reporter molecule will preferably be inhibited. In such cases, it is advantageous for the selection of cells in which the interaction has occurred for the expression of the reporter molecule to be toxic to the cell. For example, the CYH2 gene encodes a product which is lethal to yeast cells in the presence of the drug cycloheximide or the LYS2 gene which confers lethality in the presence of the drug α-aminoadipate (α-AA). In this case, only those cells in which the interaction between the binding partners has occurred will survive selection. Alternatively, if the FIS partner activates activity of the other binding partner in vitro, it is preferable for expression of the reporter molecule to be activated by the interaction between the binding partners. In such cases, it is advantageous for the selection of cells in which the interaction has occurred for the expression of the reporter molecule to encode resistance to a toxic compound, for example an antibiotic compound or herbicide. As with other embodiments described herein, only those cells in which the interaction between the binding partners has occurred will survive selection on the selective medium.

In the case of protein-based binding partners which interact with the FIS partner, the expression of the reporter molecule may be linked to the interaction between the binding partners by expressing both binding partners as fusion polypeptides with different regions derived from a known transcription factor, such that their interaction reconstitutes a functional transcription factor which is capable of regulating expression of the reporter molecule in the cell. As with the other embodiments described herein, the selection of reporter molecule and the selection means will depend upon whether or not the interaction between the binding partners has a positive or negative effect on expression of a structural gene in the cell to which the interaction is operably connected.

Examples of suitable reporter genes include but are not limited to HIS3 (Larson et al., 1996; Condorelli et al., 1996; Hsu et al., 1991; and Osada et al., 1995) and LEU2 (Mahajan et al., 1996) the protein products of which allow cells expressing these reporter genes to survive on appropriate cell culture medium. Conversely, the reporter gene is the URA3 gene, wherein URA3 expression is toxic to a cell expressing this gene, in the presence of the drug 5-fluoro-orotic acid (5FOA). Other counterselectable reporter genes include CYH1 and LYS2, which confer lethality in the presence of the drugs cycloheximide and a-aminoadipate (α-AA), respectively.

The cells used to perform this embodiment may be any cell capable of supporting the expression of exogenous DNA, such as a bacterial cell, insect cell, yeast cell, mammalian cell or plant cell. In a particularly preferred embodiment of the invention, the cell is a bacterial cell, mammalian cell or a yeast cell. In a particularly preferred embodiment of the invention, the cell is a yeast cell.

The promoter which is used to regulate expression of the binding partners and/or the reporter molecule must be operably in the cell line used. In the case of yeast and/or bacterial cells, it is particularly preferred that the promoter is selected from the list comprising GAL1, CUP1, PGK1, ADH2, PHO5, PRB1, GUT1, SP013, ADH1, CMV, SV40 or T7 promoter sequences. Wherein the promoter is intended to regulate expression of the reporter molecule, it is further preferred that said promoter include one or more recognition sequences for the binding of a DNA binding domain derived from a transcription factor, for example a GAL4 binding site or LexA operator sequence.

Any standard means may be used to introduce the nucleic acid molecules which encode the binding partners and reporter molecule into the cell, including cell mating, transformation or transfection procedures. The nucleotide sequences encoding the binding partners may be each contained within a separate genetic construct and introduced into the cell together or by sequential transformation. Alternatively, these nucleotide sequences may be introduced into separate populations of host cells which are subsequently mated and those cell populations containing both nucleotide sequences selected on media permitting growth of host cells successfully transformed with both nucleic acid molecules. Alternatively, these nucleotide sequences may be contained on a single genetic construct and introduced into the host cell population in a single step.

Cells in which the interaction between the binding partners has occurred are selected and the nucleic acid molecule which encodes the other partner (i.e. the non-FIS partner) may be recovered from the cell and the nucleotide sequence and derived amino acid sequence encoded therefor are determined using standard procedures. Techniques for such methods are described, for example by Ausubel et al (1987 et seq), amongst others.

Accordingly, a still further aspect of the present invention contemplates peptides, oligopeptides and polypeptides and isolated nucleic acid molecules identified by the method of the present invention.

The isolated nucleotide sequences which encode nucleic acid binding partners capable of interacting with a FIS partner may be expressed directly in a transgenic plant cell, tissue or organ under the control of a suitable promoter sequence, to confer autonomous or pseudogamous phenotypes thereon. Because the FIS polypeptide is a negative regulator of autonomous seed development, these non-FIS partners are likely to represent DNA-binding sites in the promoter region of a gene the expression of which is required for seed development to occur. Accordingly, removal of the FIS-binding domains from such genetic sequences, such as by expressing the genetic sequence under the control of a heterologous promoter which is not recognised by FIS will confer the autonomous seed phenotype on the cell. Similarly, in the case of polypeptide non-FIS partners, mutagenesis to remove the FIS recognition domains therefrom will also remove or reduce the ability of the FIS polypeptide to inhibit, or otherwise reduce autonomous seed development in the plant.

A further aspect of the invention extends to a monoclonal or polyclonal antibody molecule which is capable of binding to a FIS polypeptide or an epitope thereof.

Standard methods may be used to prepare the antibodies. By using a FIS peptide, oligopeptide or polypeptide described herein, polyclonal antisera or monoclonal antibodies can be made using standard methods. For example, a mammal, (e.g., a mouse, hamster, or rabbit) can be immunized with an immunogenic form of the FIS peptide, oligopeptide or polypeptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titres in plasma or serum. Standard ELISA or other immunoassay can be used with the immunogen as antigen to assess the levels of antibodies. Following immunization, antisera can be obtained and, if desired IgG molecules correspond to the polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art. For example, the hybridoma technique originally developed by Kohler and Milstein (1975) as well as other techniques such as the human B-cell hybridoma technique (Kozbor et al., 1983), the EBV-hybridoma technique to produce human monoclonal antibodies (Cole et al., 1985; Roder, 1986), and screening of combinatorial antibody libraries (Huse et al., 1989). Hybridoma cells can be screened immunochemically for production of antibodies which are specifically reactive with the peptide and monoclonal antibodies isolated.

As with all immunogenic compositions for eliciting antibodies, the immunogenically effective amounts of the peptides of the invention must be determined empirically. Factors to be considered include the immunogenicity of the native peptide, whether or not the peptide will be complexed with or covalently attached to an adjuvant or carrier protein or other carrier and route of administration for the composition, i.e. intravenous, intramuscular, subcutaneous, etc., and the number of immunizing doses to be administered. Such factors are known in the vaccine art and it is well within the skill of immunologists to make such determinations without undue experimentation.

Antibodies can be fragmented using conventional techniques and the fragments screened for utility in the same manner as described above for whole antibodies. For example, F(ab′)2 fragments can be generated by treating antibody with pepsin. The resulting F(ab′)2 fragment can be treated to reduce disulfide bridges to produce Fab′ fragments.

It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody.

The polyclonal, monoclonal or chimeric monoclonal antibodies can be used to detect the peptides of the invention, parts thereof, analogues, or homologues in various biological materials, for example they can be used in an ELISA, radioimmunoassay or histochemical tests.

### EXAMPLE 1

### Plant Material and Growth Conditions

The wild type Colombia, C24, Landsberg erecta, pistillata2 (pi2) mutant, and CHII were provided by Arabidopsis Biological Resource Center (Ohio State University, Ohio., USA.). DSG line and AC1line were provided by Dr. Sundaresan, Singapore.

Arabidopsis thaliana was grown either in pots containing a mixture of 50% (v/v) sand and 50% (v/v) compost, or aseptically in petri dishes containing a modified Murashige and Skoog (MS) media (Langridge, 1957). All plants were grown in artificially lit cabinets at 23° C., under long day (16 h light, 8 h dark), or continuous light (24 h light) conditions at a light intensity of 200 mmol m⁻² sec⁻¹.

### EXAMPLE 2

### A Visual Screen for Determining Autonomous Endosperm Development in Plants

I. Background

A visual screen was developed to determine whether a particular plant has the capacity for autonomous or pseudogamous development of seeds and seed-like structures. Our visual genetic screen is based on the difference in silique length between sterile (short silique) and fertile (long silique) Arabidopsis thaliana plants.

Arabidopsis thaliana is a self-fertilising hermaphrodite plant. The fused carpel or silique is surrounded by the male sexual organs consisting of six stamens topped by anthers that release pollen during anthesis. In self-fertile plants, anthesis and pollination is complete even before the flowers are completely opened. As fertilisation takes place and seeds are formed, the siliques elongate about five-fold giving rise to full-length seed pods. In the absence of seed formation, the siliques remain short.

Mutants of Arabidopsis thaliana are known which have either impaired male structural organs (for example, the stamenless or antherless mutants) or microspore development (such as the pollenless mutant). In particular, the recessive mutation pistillata (pi) produces a mutant plant when expressed in the homozygous state (i.e. pi/pi) which is devoid of petals and stamens, has short siliques, but undiminished female-fertility. When exogenous pollen is used to pollinate the stigma of the pi/pi mutant, siliques are elongated to the level seen in wild-type plants.

Material derived from such an approach may comprise plants capable of dominant or recessive autonomous endosperm formation, or partially-dominant or recessive pseudogamous endosperm formation. These may be distinguished from each other according to the following experimental design.

II. Experimental Design

A. Visual Screen for Partially Dominant and Recessive Autonomous Endosperm Development in Plants

This screen comprised the mutagenesis of plants containing the pistillata mutation and the subsequent selection of those plants in which silique elongation was observed in the absence of fertilization by a pollen donor. Plants which were putatively characterised as being capable of autonomous endosperm development were identified by their ability to produce elongated siliques in the absence of fertilisation, without concomitant reversion of the male reproductive apparatus.

Heterozygous PI/pi seeds were made by pollinating a female pi/pi homozygote with pollen from a wild-type homozygous PI/PI plant. The Pi/pi heterozygous seeds produced from this cross were then mutagenised using ethyl methane sulfonate (EMS). The M1 plants were grown and self-fertilised and M2 seeds were harvested and planted.

Four types of plants, heterozygous PI/pi (fully-fertile), homozygous wild-type PI/PI (fully-fertile), homozygous recessive pi/pi (male-sterile amphimictic plants having only short siliques) and homozygous recessive pi/pi apo/apo(male-sterile soft-seeded plants having elongated siliques) were present in the M2 generation. The pi/pi plants do not produce normal stamens or petals and were readily distinguished from the fully-fertile plants.

Those plants which were self-fertile with normal stamens and petals (i.e. PI/PI and PI/pi plants) were uprooted and discarded as soon as they were identified. Among the pi/pi homozygotes, those plants which are putative soft-seeded mutants were identified as stamenless plants having long siliques.

B. Visual Screen for Partially-dominant and Recessive Pseudogamous Endosperm Development

Plants (pi/pi) were subjected to a pseudogamy test as follows: The pi/pi M2 plants were pollinated with pollen derived from wild type PI/PI plants. Silique elongation was monitored in the pollen recipients to ascertain that the crosses were successful. Seeds were harvested, planted and the resulting plants were screened for the maternally-derived (pi/pi) phenotype which, following such cross-pollination, is indicative of partially-dominant or recessive pseudogamous endosperm development having occurred. Absent complete penetrance of the soft-seeded phenotype, dominant pseudogamous mutants are also detected in this screen.

C. Visual Screen for Dominant Pseudogamous Endosperm Development

To distinguish dominant pseudogamous mutants from partially-dominant and recessive pseudogamous mutant plants, pi/pi M1 plants were screened directly after mutagenesis for sectors having elongated siliques. To test for pseudogamy, pi/pi plants after mutagenesis were crossed with wild-type PI/PI plants as described for recessive autonomous endosperm development. Silique elongation was monitored in the pollen recipients to ascertain that the crosses were successful. Seeds were harvested, planted and the resulting plants were screened for the maternally-derived (pi/pi) phenotype which, following such cross-pollination, is indicative of dominant pseudogamous endosperm development having occurred.

### EXAMPLE 3

### Mutagenesis, Mutant Identification and Analysis

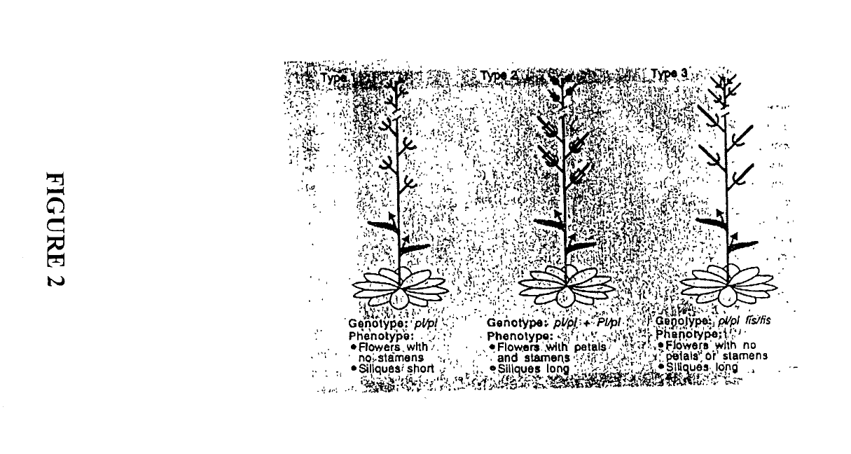

Heterozygous PI/pi seeds were generated by pollinating a homozygous pi/pi mutant plant with pollen from a wild-type PI/PI plant. For each mutagenesis, 2 gram of F1 seed (PI/pi) was mutagenized as described previously (Chaudhury et al., 1994) and germinated in pots to produce the M1 generation. The M1 plants were allowed to self-fertilize and set seed. Seed from each pot of the M1 plants were harvested separately by collecting at least 10 mature siliques from each plant to ensure that sufficient seeds were obtained from each M1 plant. In the M2 population, 1/4 of the progeny plants were homozygous for the pistillata mutation (pi/pi). Fully-fertile PI/pi and PI/PI plants were identified by the presence of petals and stamens and were removed. Mutants were detected in the pi/pi population, on the basis of elongation of siliques without formation of stamens (FIG. 2).

I. Identification and Analysis of Mutants Showing Partially Dominant and Recessive Autonomous Endosperm Development

All EMS-generated mutants were crossed with wild-type plants and the F1 plants were selfed to produce F2 seeds, in order to observe dominant, recessive and partially-dominant mutations in the next generation.

In the screen described herein for autonomous mutants, a total of six mutants were identified in which silique elongation and seed development was observed in the absence of pollination. These mutants were designated as fis (i.e. fertilisation independent seed) mutants. More particularly, these six mutants fell into three complementation groups, designated fis1, fis2 and fis3. Three of the six mutants are allelic to fis2 and were designated fis2-1, fis2-3 and fis2-4.

The six fis mutants obtained so far are from different M1 seed families and thus represent independent mutations. The developmental analyses done so far has been carried out using plants obtained from a primary mutant screen.

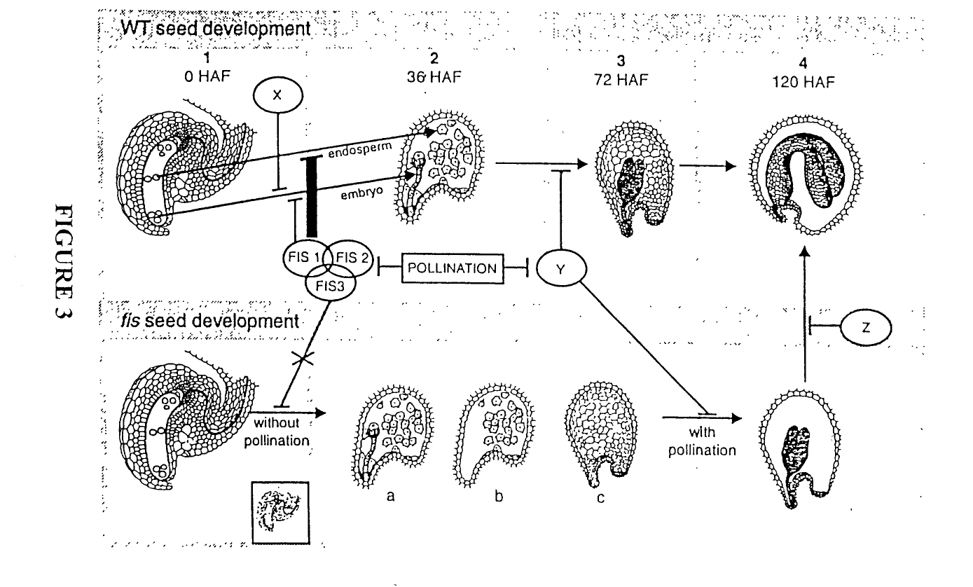

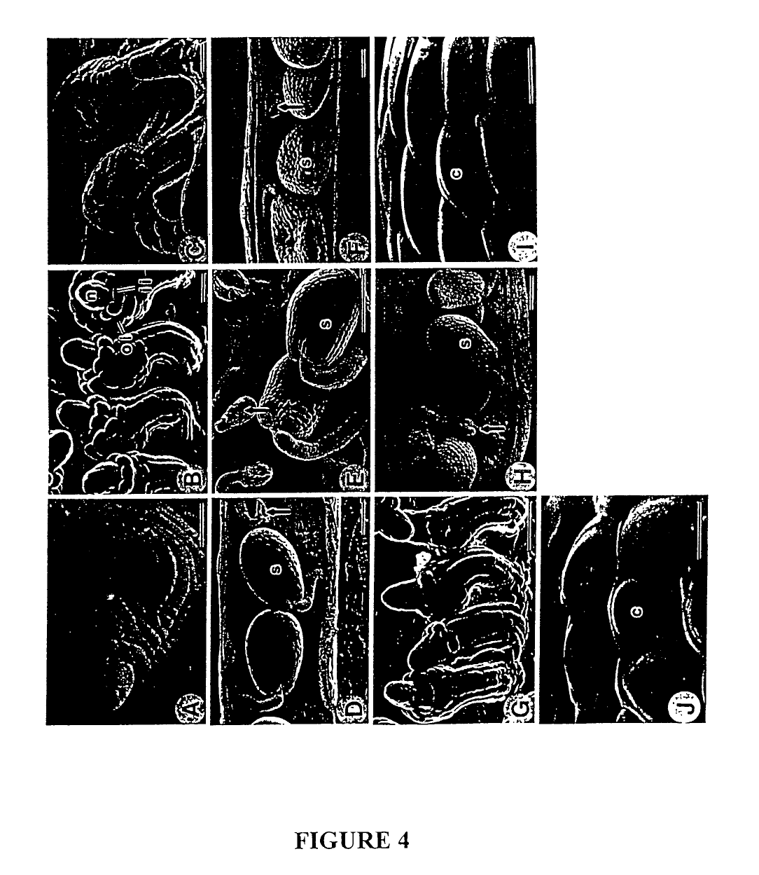

A comparison of seed morphology and development in the fis mutants, compared to wild-type Arabidopsis thaliana plants is presented in FIGS. 3, 4 and X.

A. Seed Morphology and Development in the Absence of Fertilisation

Based on the analyses of seed size and shape by scanning electron microscopy (SEM) studies, the seed morphology and development are not significantly altered in the mutants compared to wild-type seeds. Detailed sectioning and Nomarski optics studies have been done in one of these mutants.

In unpollinated heterozygotes of the fis mutants, one-third to one-half of the ovules in the elongated siliques were transformed into seed-like structures resembling normal, sexually produced seed in external morphology and size. Endosperm cells develop normally and aborted embryo-like structures develop. The seeds of such plants were initially white, however became shrivelled and brown as they matured. Accordingly, such mutants exhibit an autonomous partial seed (APS) phenotype and are at least capable of autonomous endosperm development. In control pi/pi plants, no endosperm or embryo-like structures were formed.

B. Seed Morphology and Development Following Fertilisation

Fertilized ovules of pi/pi plants developed into seeds. All sexually-fertilized seeds from wild-type plants turn green and mature after pollination, whereas seeds from pollinated FIS/fis heterozygotes contained green (mature) and white (embryo-arrested seed) at a 1:1 ratio. The fis ovules were similar to FIS ovules in early stages of ovule development. Both inner and outer integuments and the nucellar tissues of the fis mutants were indistinguishable from those of FIS plants.

When siliques containing the white seed were pollinated, some seeds developed which became green and eventually brown. Other seeds remain white but develop embryos which are clearly past the globular stage. This result suggests that the mutation conferring the APS trait is co-dominant. We are currently investigating the possibility that the partially-developed embryos are pseudogamous.

In one mutant at least, analysis of the progeny suggest that the white seed phenotype is controlled by the female gamete, rather than the sporophyte. The gametophytic control may be indicative of diplospory in this mutant. This question may be resolved by following the transmission of the mutant phenotype via the pollen. In the instant case, such an analysis is possible because the M2 seed were obtained in families and the gametophytic mutants may be identified in fertile plants.

Embryo sac, embryo, and endosperm development in ovules from the fis mutants were compared with those of ovules of the cogenic Ler-FIS plants. In pi/pi ovules, no embryo or endosperm cells were seen. Three days after pollination of the pi/pi plant with pollen from a PI/PI plant, the ovules contained an embryo and free nuclear endosperm cells, and each ovule had expanded to the size of the mature seed. In the mutant ovules from a FIS2/fis2 heterozygous plant, the ovule development was equivalent to the development of pi/pi ovules 3 days after pollination, and endosperm cells occasionally were accompanied by an embryo-like structure at the micropylar end (FIG. 4).

When the fis2/fis2 homozygous mutant plants were pollinated with pollen from a FIS/FIS plant, embryos developed further than they did in the unpollinated fis2/fis2 plants.

Homozygous fis2 plants were pollinated with pollen from a FIS/FIS plant homozygous for a 35S-GUS reporter gene. The resulting torpedo-stage embryos were stained to detect the product of the GUS gene. All of the embryos resulting from self-pollination of the FIS/FIS 35S-GUS/35S-GUS plant stained blue, as did the embryos resulting from a pollination of a pi/pi FIS/FIS plant with pollen from a 35S-GUS/35S-GUS plant. In contrast, when 35S-GUS pollen was used to pollinate fis2/fis2 homozygotes, the resulting torpedo stage embryos were either GUS-positive or GUS-negative, suggesting that both zygotic and maternal embryos were present. The presence of GUS sequences in the blue embryos and their absence in the white embryos has been confirmed by PCR using primers from the GUS genes.

After fertilization, the outer integuments of the Arabidopsis wild-type ovule develop polygonal structures with a central elevation called the columella (Mansfield, 1994). These structures were not seen in unfertilized ovules that did not develop any mature seed characters before they atrophied. Although the fis seeds were not fertilized, they did form the columella in the outer integument cells, and they were indistinguishable from normal zygotic seeds before they shrivelled.

C. Ploidy of the Endosperm

The ploidy of the endosperm cells from fis2 mutant was determined by measuring the fluorescence intensity of nuclei in 4′,6-diamidino-2-phenylindole-stained sections. The average brightness of autonomous fis2 endosperm nuclei was found to be 79.4±14.4 (n=40), and that of wild-type control nuclei was 108±23.1 (n=42). The background value was 35.5±6.2. The results are consistent with the autonomous endosperm being diploid in contrast to the triploid condition of the sexual endosperm nuclei.

II. Identification and Analysis of Pseudogamous Mutants

Approximately 15,000 homozygous recessive pi/pi M2 plants were bulk-pollinated with pollen from L. erecta parent and 90,000 plants were screened for maternal pi/pi phenotype as an indication of pseudogamy.

Approximately 0.1% of plants produced progeny having the recessive maternal phenotype. The possibility existed that these plants may be the result of an extremely rare self-pollination in plants having a very low level of reversion of the pistillata allele to wild-type. As a consequence, those progeny having the recessive maternal phenotype were progeny-tested in the next generation. These progeny are analysed as described supra and pseudogamous mutants are retained and analysed further.

III. Further Analysis of Mutants

Embryo Sac Development

The autonomous and pseudogamous mutants obtained to date were analysed further with respect to determining the nature of embryo sac development therein. We have developed a clearing technique which enables female meiosis and embryo sac development to be observed in wild-type plants and this technology is also used to analyse female meiosis and embryo sac development in each of the mutants.

The present inventors observed an embryo sac with a two cell embryo in sections of fis3-2 mutant seed-like structures.

Effects of Genetic Background in Modifying Mutant Phenotypes

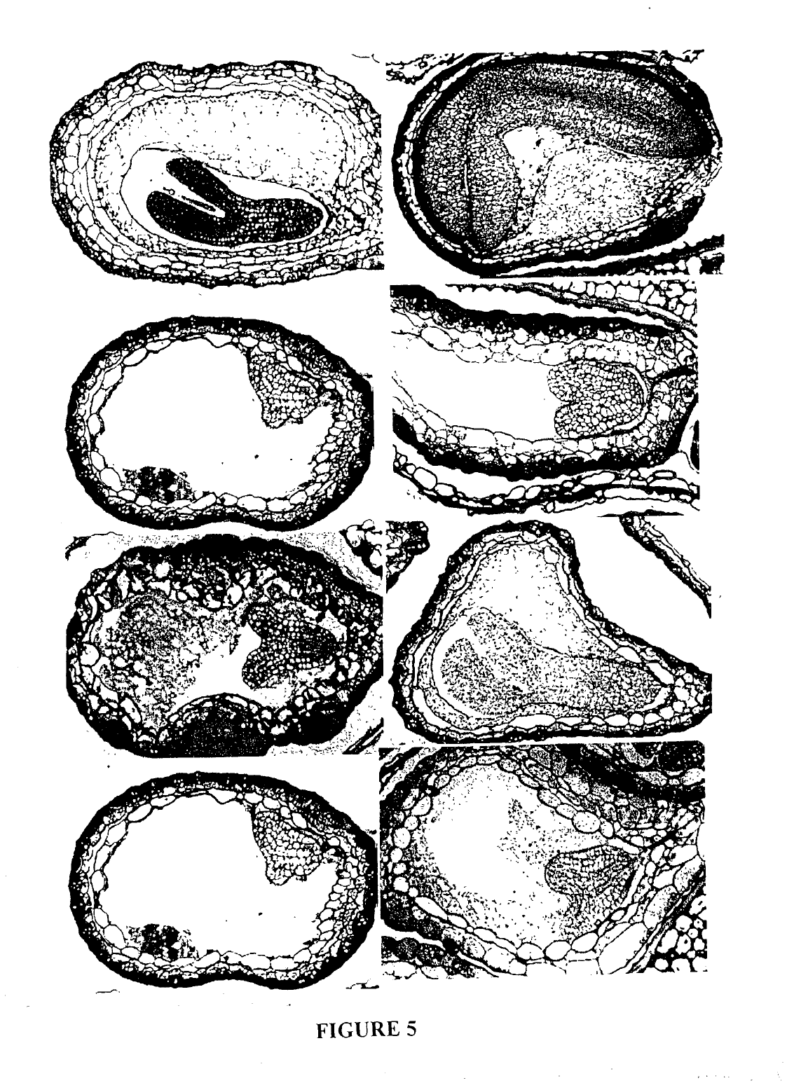

The embryos derived from the mutant embryo sacs are arrested mainly at heart stage irrespective of paternal contributions for all fis mutants in the Ler genetic background (FIG. 5, panels 1-4). In fis1, fis2-1, and fis2-2 homozygous mutants, the proportion of embryos arrested at various stages were investigated in the Ler background. In the case of fis1/fis1 homozygotes, 140/155 seeds arrested at heart stage, 4/155 seeds were not arrested, and the remaining seeds were arrested beyond the torpedo stage of development. Similar numbers were obtained for fis2-1 and fis2-2 homozygous mutants in the Ler background. However, no fis3 homozygous plants were generated (see below).

In contrast, when the fis1and fis2 mutants were crossed to the ecoptype Col, the proportion of mutant embryos in the progeny which were arrested at later stages increased, compared to that observed in the Ler background.

In particular, the proportion of mutant seeds with torpedo embryo or beyond was determined for the mature seeds of Col×fis1, Col×fis2 and Col×fis3 crosses. In the progeny of the Col×fis1 cross, the proportion of homozygous fis1 mutant seeds with embryos arrested at the torpedo stage or beyond was 10.5% in the F2 generation [i.e. (Col×fis1) F2] compared to only 3.2% in the Ler background. In the progeny of the Col ×fis2 cross, the proportion of homozygous fis2 mutant seeds with embryos arrested at the torpedo stage or beyond was 15% in the F2 generation [i.e. (Col×fis2) F2] compared to only 4.5% in the Ler background. In the progeny of the Col×fis3 cross, the proportion of heterozygous fis3 mutant seeds with embryos arrested at the torpedo stage or beyond was 4.5% in the F2 generation [i.e. (Col×fis3) F2] compared to only 2.8% in the Ler background.

Given the difference of embryo development for the fis1 and fis2 mutants between Ler and Col backgrounds, it is likely that there exists a modification system in Col that allows the mutant embryos to develop further than in Ler. To determine the genetic basis of this modification, fis2-1/fis2-1 and fis2-2/fis2-2 homozygous mutants were screened from the (Col×fis2) F2 population (FIG. 5, panels 5 and 6). Some homozygous mutants showed much better embryo development than others. For example, one (Col×fis2) F2 plant produced 42/117 wild-type looking seeds, compared to only 9/159 fis2-1/fis2-1 seeds in the Ler background. In some extreme cases we could observe up to 100% seeds looking normal in some part of the plants.

An unmodified fis1/fis1, an/an (Ler) mutant was crossed to one modified fis2-2/fis2-2 (Col) plant. From the progeny of this cross, double homozygous mutants were constructed as described above and some lines showed further embryo development (i.e. later arrest). One double mutant line produced up to 40/195 wild type looking seed. These data suggest that fis1 and fis2 may share the same modification system.

To investigate the role of the modification system in embryo development, the modified seeds were sectioned and compared to the same stage of the unmodified fis2-1 in the Ler ecotype background. Data indicated that endosperm cellularisation in modified seeds was similar to that of wild-type seeds, while most fis2-1 seeds in the Lerecotype lacked endosperm cellularisation or were only partially cellularised. Without being bound by any theory or mode of action, these data suggest that the modification system may involve an endosperm cellularisation process.

In order to understand the influence of the modification system on the seedlings derived from the mutant seeds, we germinated the arrested seeds from the F2 seeds from the crosses between Col and all three fis mutants. The seedlings from the arrested seeds displayed a wide range of morphological phenotypes. The seedlings can be divided in three groups based on the ability to regenerate into viable plants, as follows:

(i) normal looking seedlings that show no obvious difference from wild type;

(ii) seedlings that display abnormalities at early stages of development and later become viable and form wild type looking plants; and

(iii) morphologically-deformed seedlings that can not develop into viable seedlings.

In this grouping, type (ii) seedlings have fewer abnormalities than type (iii) seedlings, particularly in respect of the cotyledons and the bottom rosette leaves which usually become thicker, longer and deformed in type (iii) plants. The upper rosette leaves were gradually restored to wild type appearance in type (ii) plants. The upper part of type (ii) plants is completely normal and can produce flowers and seeds. Type (iii) seedlings are dramatically deformed with accumulation of anthocyanins in the thickened cotyledon, an no green rosette leaves form in these plants, possibly explaining why these seedlings do not develop into viable plants.

To correlate seed phenotype to the stage of embryo arrest, we arranged the modified fis2-1 homozygous mutant seeds into three groups, as follows:

(i) normal looking mutant seeds;

(ii) seeds with torpedo or further developed embryo; and

(iii) completely flat seeds or seeds with heart stage embryo.

Type (i) seeds produced only wild type plants and 80% of these seed germinated. Type (ii) seeds produced all three types of seedlings listed supra, in the ratios of 80% wild type seedlings; 15% type (ii) seedlings; and 3% type (iii) seedlings. Type (iii) seeds germinated at a rate of 9/120 seeds and only produced Type (iii) non-viable seedlings.

Studies of Homozygous Mutant Plants

In spite of several attempts to identify homozygous mutants for both the fis3-1 and fis3-2 mutant alleles, no homozygote was obtained in Ler or Col ecotype backgrounds. In contrast, it is easy to obtain fis1 and fis2 homozygotes for all fis2 alleles. In an attempt to generate fis3-1 and fis3-2 homozygous mutants, about 2,000 arrested seeds for each of (Col×fis3-1)F1 and (Col×fis3-2) F1 plants were germinated on MS plates. Those seeds were derived from mutant embryo sacs which had been fertilized by either wild type or mutant pollen with equal chance as the mutation does not affect the fertility of pollen. Theoretically, FIS3/fis3 and fis3/fis3 should be obtained with equal numbers among the germinated plants if the fis3 mutation does not affect embryo development. However, for fis3-1 we could obtain only 28 heterozygous plants and for fis3-2, we could only obtain 23 heterozygous, thereby showing the conditional lethality of the mutation in fis3-1/fis3-1 and fis3-2/fis3-2 homozygotes. In contrast, fis1 and fis2 homozygotes accounted for 50% of the total surviving plants in similar screening in the Col×fis1 and Col×fis2 crosses. These data suggest that the FIS3 gene may have a function in the embryo.

Gene Interactions

Double mutant studies are important genetic strategies to define independent pathways of gene action. If two genes act in the same pathway, the double mutant phenotype is often the same as the phenotype of the single mutant, in which case the gene of the single mutant is epistatic over the other gene which is mutated in the double mutant. However, the effect of each allele in a double mutant may be enhanced or even synergistic, giving rise to a qualitatively novel phenotype in the double mutant compared to what would be expected from the parental phenotypes. Double mutants are produced by standard genetic procedures which are well-known in the art.

Because the APS phenotype obtained in at least one of our fis mutants appears to be co-dominant from the point of view of autonomous endosperm development, double mutants are produced which comprise combinations between this mutant and the other five single mutants described herein, to clarify the pathways that control autonomous seed production and to produce mutant plants having a higher degree of penetrance of the autonomous seed phenotype. Double mutants between each of the other fis mutants are also produced.

In particular, a double an/an, fis1/fis1 mutant was crossed to the Ds-induced fis2-2/fis2-2 mutant in a Col background (i.e. a fis2-2fis2-2 modified mutant). The F1 plants with 75% mutant seeds were harvested and germinated on MS plates with kanamycin selection to select for the fis2-2 allele. Because these plants were kanamycin resistant, they must at least contain one copy of fis2-2 gene. The surviving plants were also screened to isolate those showing the an/an marker phenotype, and the DNA from these plants was sequenced to select those homozygous for the fis1 mutation. To detect homozygous fis2-2 mutants, we designed three primers for use in PCR screening as follows:

(i) a first pair of primers derived from the Ds-interrupted FIS2 sequence in the fis2-2 mutant, which in use provides a positive PCR product only when there is no Ds insertion; and

(ii) a second pair of primers, comprising a Ds-specific primer derived from the nucleotide sequence of Ds and a second primer derived from the FIS2 sequence in the fis2-2 mutant, which in use provides a positive PCR product when the fis2-2 mutant allele is present.

This screening strategy was used to generate three fis1/fis2-2 double homozygous plants. There are no morphological abnormaties in these double mutants except in the an/an selection marker. After emasculation, these plants still produced seeds similar to those observed for the single fis1 or fis2 mutant plants. In the double homozygotes, the seeds were arrested in the same way as for the fis2-2/fis2-2 modified mutant (FIG. 5, panels 7 and 8). In the F2 generation, some plants exhibited a lesser degree of modification than the fis2-2/fis2-2 modified mutant, producingmainly seeds having a heart stage embryo.

Conditionality of the Mutant Phenotype

The possibility that the autonomous development of seeds in the fis mutant is influenced by environmental conditions is tested by growing the six fis mutants at a constant temperature of 16° C. and under photoperiods comprising either 8 hr light or 16 hr light, compared to the conditions under which the mutations were first-detected (i.e. 22° C. under continuous light). Plants having a higher degree of penetrance of the autonomous seed phenotype are retained for further analysis.

Gene Dosage Effects

In many of the autonomous fis mutants described herein, sexual transmission of the mutant fis allele following cross-pollination with a pollen donor may occur at a low frequency, indicating a degree of female sterility is associated with the mutation. Heterozygous plants are isolated by screening for the mutation in fertile plants. The heterozygous plants are then used to construct genetic lines of plants in which the mutation is in homozygous condition, such that all seeds produced therefrom are autonomous. Genetic lines in which the level of penetrance of autonomous seed production is increased are retained for further analysis.

### EXAMPLE 4

### Mapping of FIS Alleles

To map the FIS loci, pollen from each of the FIS/fis PI/PI plants was used to pollinate W100F, a male-fertile derivative of W100 that contains 10 morphological mutations distributed on the arms of the five Arabidopsis chromosomes (Koornneef et al, 1987). Among the F2 progeny of FIS/fis W100F/+, plants which were homozygous for the different recessive morphological mutations were scored for FIS/FIS (all seeds in the siliques were normal) and for FIS/fis (the siliques contained a mixture of fully developed and embryo-arrested seeds).

I. The FIS1 allele

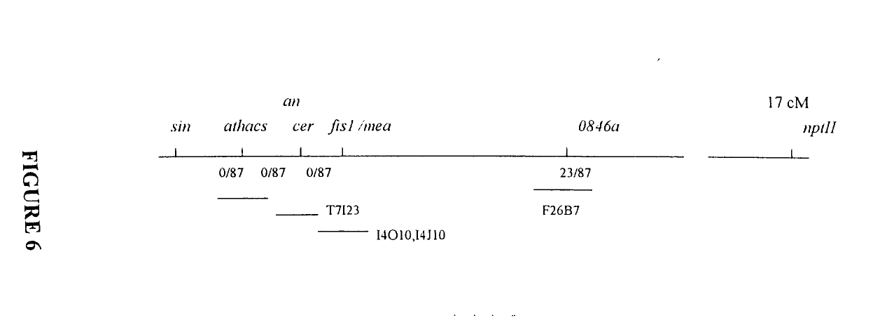

Genetic data showed that the morphological marker an was closely linked to the fis1 allele. The genetic distance between an and FIS1 is 1 cM (FIG. 6). As FIS1 was localized to the end of chromosome 1, two flanking markers were used to further map the FIS1 gene.

One such marker comprised the kanamycin-resistance gene NPTII, which is present in this region of chromosome 1 of a genetic line of Arabidopsis thaliana ecotype No-0 designated E12, as part of a genetic construct containing the Ds transposable element. The E12 line was crossed to the fis1 mutant and F1 progeny were back-crossed to wild-type Arabidopsis thaliana ecotype Landsberg erecta (Ler). Recombinants between fis1 and NPTII were selected from the backcrossed F1 lines. Following this approach, the genetic distance between fis1 and NPTII was determined to be 17 cM (FIG. 6).

To identify the closest molecular marker to the FIS1 gene, SSLP markers from contiguous BAC clones in the region of the morphological marker an were designed, based on the released sequence data from Arabidopsis data base.

The SSLP marker designated F26B7 (FIG. 6) was used first to test recombinants between the FIS1 and NPTII genes. From 87 plants produced from such recombination events, 23 plants were identified in which a crossover had occurred between F26B7 and the FIS1 gene, a recombination frequency of 26.4%.

The SSLP markers athacs and the left-end and right-end rescue fragments derived from the BAC clone T7123 were also used to test these 87 plants. No plants were identified in which a crossover had occurred between FIS1 and the SSLP markers, indicating that FIS1 is tightly linked to these markers on chromosome 1 (FIG. 6).

The BAC clone T5P2 which contains athacs, the BAC clone T7123 and the BAC clone F26B7 map to the same contiguous region on chromosome 1. Accordingly, data indicated that the FIS1 gene was located either within the BAC clone T7123 or within the BAC clone which maps immediately to the left of T7123 (FIG. 6).

The MEDEA (syn. MEA) gene described by Grossniklaus et al (1998) was shown to map in this region of chromosome 1. Plants expressing the mea phenotype exhibit embryo lethality Grossniklaus et al (1998), however do not exhibit autonomous seed development. The mea mutant is a Ds-tagged gametophytic maternal mutant. To determine how closely the MEA gene mapped to the FIS1 gene on chromosome 1, a PCR-generated probe derived from the nucleotide sequence of the MEA gene was hybridized to clones on an IGF filter. Five positive clones were identified, which mapped to the left of the BAC clone T7123 (FIG. 6), indicating a tight linkage.

DNA derived from the fis1 homozygous mutant was also sequenced using MEA gene primers and a single base change was found in fis1 mutant compared to the wild-type MEA gene sequence. This base change introduced a translation stop codon in the 5′-region of the open reading frame of the MEA gene, thereby resulting in early termination of translation and the synthesis of a truncated polypeptide. These data indicate that the fis1 mutant gene is an allele of the MEA gene. However, the different phenotype of the fis1 mutant compared to the mea mutant, indicates that the point mutation in fis1 is critical to reduce expression of the wild-type MEA/FIS1 gene to a biologically inactive level which is sufficient to facilitate autonomous seed development.

I. The FIS2 Alleles

Mapping studies on the FIS2 gene utilised the fis2-1 mutant line where appropriate.

The fis2-py recombination frequency of 9,28±1.56 (map distance of 10.26; n=345) and the fis2-er recombination frequency of 13.07±2.73 (map distance of 15.14; n=153) positioned fis2 between er and py on chromosome 2.

The heterozygous FIS2/fis2 was crossed to wild-type Arabidopsis thaliana ecotype Colombia (Cross No.1) or CHII (Cross No. 2) and the F2 progeny were obtained. For each selected individual F2 plant derived from these crosses, a pool of F3 plants was grown to facilitate determination of the genotype of the corresponding F2 plant. In the F2 population derived from Cross No. 1, er/er FIS2/fis2 recombinants were isolated and allowed to self-fertilize. In the F2 population derived from Cross No. 2, FIS2/fis2 as/as plants were isolated and allowed to self-fertilize.

DNA from the F3 pools was prepared for RFLP analysis. Three types of RFLP probes were used in this analysis. Clones such as mi277, m323, and ve017 which appear on the RI map, the left and right ends of YAC clones and fragments derived from cosmid clones or BAC clones were used. Total DNA extraction and DNA gel blot analysis were performed as described by Church and Gilbert (1984).

The RFLP markers ve017, mi277 and m323 were mapped relative to the ER, FIS2 and as loci using the recombinant F2 plants er/er FIS2/fis2 and FIS2/fis2 as/as. Marker ve017 mapped between AS and FIS2 genes. Of 8 plants tested, five showed a recombination break point in the FIS2-ve017 interval. On the other hand, out of 65 er/er FIS2/fis2 plants tested, 10 plants had a recombination break points in the mi277-FIS2 interval and 5 plants had a recombination break point in the m323-FIS2 intervals. These data indicate that the markers mi227 and m323 map on the ER-proximal side of FIS2, in the order ER-mi277-m323-FIS2.

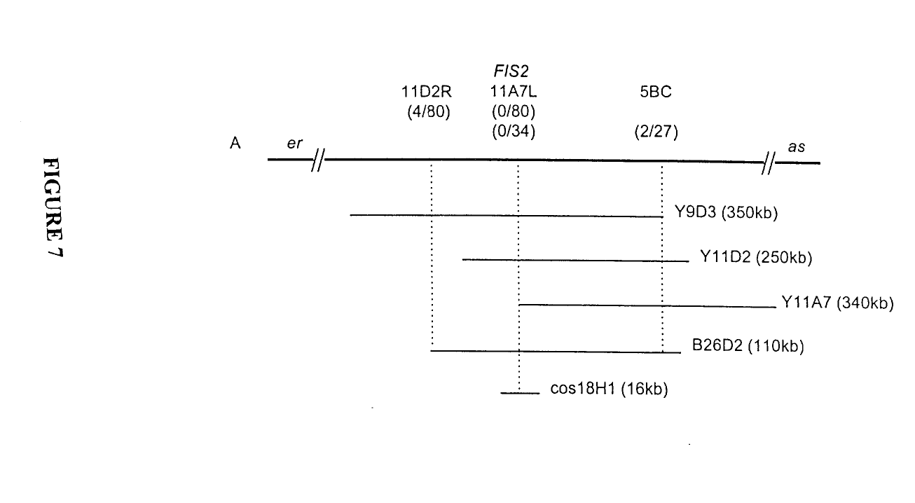

Based on a map of contiguous YAC clones for chromosome 2, the YAC clone designated Y9D3 (FIG. 7) was selected and its left and right ends were rescued and used as RFLP markers to test for linkage to the FIS2 locus in the F2 population. The Y9D3 left end-FIS2 interval showed no recombination break point out of 65 er/er FIS2/fis2 plants tested. However, a recombination break point was observed in 3 plants out of 9 FIS2/fis2 as/as F2 plants. These data indicate that the left-end of the YAC clone Y9D3 maps on the as proximal side of FIS2 (FIG. 7).

Using the Y9D3 left-end as a probe, two other YAC clones, designated Y11D2 and Y11A7 in FIG. 7, were isolated from the same YAC library. The Y11D2 right-end and the Y11A7 left-end were used as RFLP markers to test their position on chromosome 2 relative to the FIS2 gene. The Y11D2 right-end mapped on the er proximal side of FIS2, whilst the Y11A7 left-end showed no recombination break point in its interval with. These data indicate that the Y11A7 left-end is tightly linked to the FIS2 gene (FIG. 7).

I. The FIS3 Allele

The FIS3 gene was located on chromosome 3, between the morphological markers hy3 and gl1 (FIG. 8). The fis3 mutant was crossed to wild-type Arabidopsis thaliana ecotype Columbia, to facilitate detailed mapping. In the F2 population, 107 plants were harvested and DNA prepared. One SSLP marker, designated nga162 (FIGS. 8 and 9) was used to determine that the nga162 marker was about 6 cM north of the FIS3 gene. An even closer RFLP marker, designated ve039 (syn. ve039) was identified which mapped cM north of the FIS3 gene (FIGS. 8 and 9). Analysis of the F2 population from a cross between the triple mutant hy/hy FIS3/fis3 gl1/gl1 and wild-type Columbia and in particular, analysis of the recombinants, for example the single-crossover mutants hy/hy FIS3/fis3 GLI/gl1 and Hy/hy FIS3/fis3 gl1/gl1, provide for accurate localization of the FIS3 gene.

A contiguous map of YAC clones and pI clones was constructed around the ve039 marker (FIG. 9). Data suggest that the FIS3 gene is present in the p1 clones MCB22 and/or MNH5 and/or the YAC clone CIC7E1, to the left of ve039.

### EXAMPLE 5

### Transposon Tagging of the FIS2 Gene

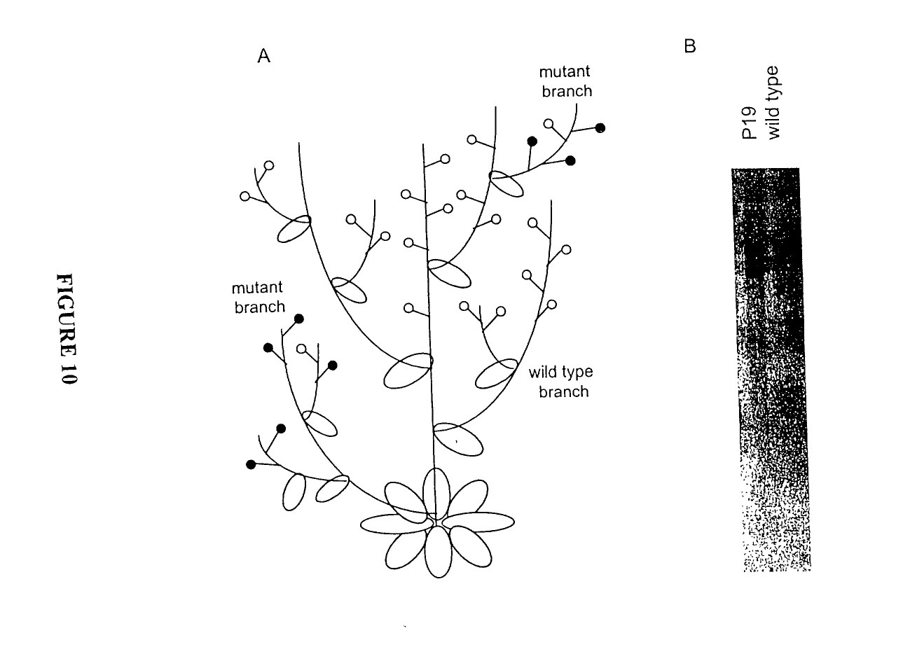

A clone containing a transposon carrying a promoterless reporter gene was also used to tag the FIS2 gene. In the DSG tagged line, the transposon was found to be closely linked to the molecular marker m323 (see Example 4). A line containing an Ac element was crossed into the DSG line fis2-2 and F1 plants were screened for sectors that show fertilization independent silique elongation and which segregate in a 1:1 ratio of normal: fis2-2 in the seeds. In the F1 of the DSG×Ac1 cross, one chimeric plant designated P19, was observed which showed both of these properties, indicating that the DSG transposon had possibly integrated into the FIS2 gene in that line (FIG. 10). The line containing the transposon inserted into the fis2 gene was designated fis2-2

### EXAMPLE 6

### Cloning the FIS2 Gene

To clone the FIS2 gene, the left-end of Y11A7 was used to screen a cosmid library provided by Dr. Neil Olszewiski (University of Minnesota, USA) and a BAC library. One 110 kb BAC clone (B26D2 in FIG. 7) and a 16 kb cosmid clone (cos18H1 in FIG. 7) were isolated, both of which contain the Fis2 gene.

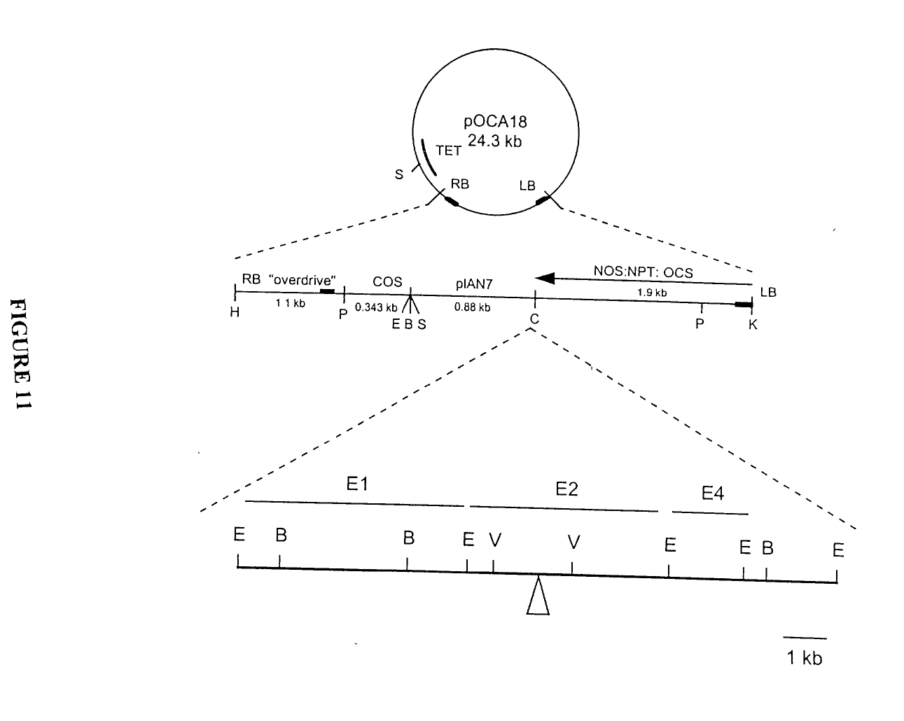

A physical map of the cosmid clone cos18H1 was obtained, using the restriction enzymes BamHI (B), EcoRI (E), and EcoRV (V) (FIG. 11).

Additionally, a bacteriophage genomic library (see Example 9) was prepared using DNA derived from the DSG-tagged fis2-2 mutant described in the preceding Example. Since the FIS2 gene mapped to the BAC clone B26D2, DSG must have transposed into a location covered by one of the sub-fragments of B26D2. The sub-fragments of B26D2 (FIG. 11) were used as probes to test the tagged mutants. DNA covered by one of the EcoRI fragments, designated E2 in FIG. 11, was interrupted by DSG. The DSG transposon also hybridized to the E2 fragment. Accordingly, the genomic library was screened using a BamHI fragment containing the DSG 5′-end and the E2 probe (see Example 9).

By sequencing the DSG-containing DNA and the corresponding wild type sequence from cosmid pOCA18H1 (FIG. 11), the position of the DSG insertion was determined to lie within the FIS2 gene.

### EXAMPLE 7

### Cosmid pOCA18H1 Complements the fis2 Mutant Phenotype

To confirm the presence of the FIS2 gene in the cosmid clone pOCA18H1 (FIG. 11), complementation tests were performed wherein this clone was introduced into the Arabidopsis thaliana fis2 mutant line.

Agrobacterium-mediated transformation of Arabidopsis thaliana root explants was performed as described by Valvekens (1988) with some modifications. Timentin was used instead of vancomycin. Bacto agar™ [0.8%(w/v)] was replaced by 0.3% (w/v) Phytoagar™. Bacto agar™ is the trademark of Difco Company and Phytoagar™ is the trademark of Sigma Chemical Company. Constructs were introduced into Agrobacterium tumefaciens strain AGL1 by the triparental mating procedures with pRK2013 as a helper plasmid (Ditta, 1980). Stability of the plasmid insert in AGL1 was tested by restriction digestion and gel electrophoresis of plasmid DNA.

Fresh overnight cultures of Agrobacterium tumefaciens strain AGL1 carrying individual plasmids were used to infect root explants derived from 4-week old Arabidopsis thaliana plants. Kanamycin-resistant transgenic plants were regenerated as described previously (Valvekens, 1988). Transformed shoots were transferred to Murashige and Skoog (MS)-containing agar, supplemented with 50 g/ml kanamycin and 100 g/ml timentin. Seeds of transgenic plants were germinated either in soil or on MS-containing agar plates supplemented with 50 g/ml kanamycin.

Cosmid pOCA18H1 (FIG. 11) was introduced into the Agrobacterium tumefaciens AGL1 strain by triparental mating using E. coli RK2013 as a helper strain. A. tumefaciens transconjugants were selected on LB containing rifampicin (50 g/ml) and tetracyclin (3.5 g/ml). Spurious rearrangements in the cointegrates were determined by re-transformation of the cosmid clone into E. coli strain D5H and restriction mapping of the plasmid DNA derived therefrom.

Arabidopsis thaliana ecotype C24 root explants were transformed with A. tumefaciens containing cosmid pOCA18H1 and regenerated as described by Valvekens et al, (1988). For each T1 plant, T2 seeds were sown on media containing kanamycin (50 g/ml) to determine the segregation ratio for kanamycin resistance. Kanamycin-resistant T2 plants were crossed to the fis2 mutant and the ratio of arrested seeds in F1 plants were scored.

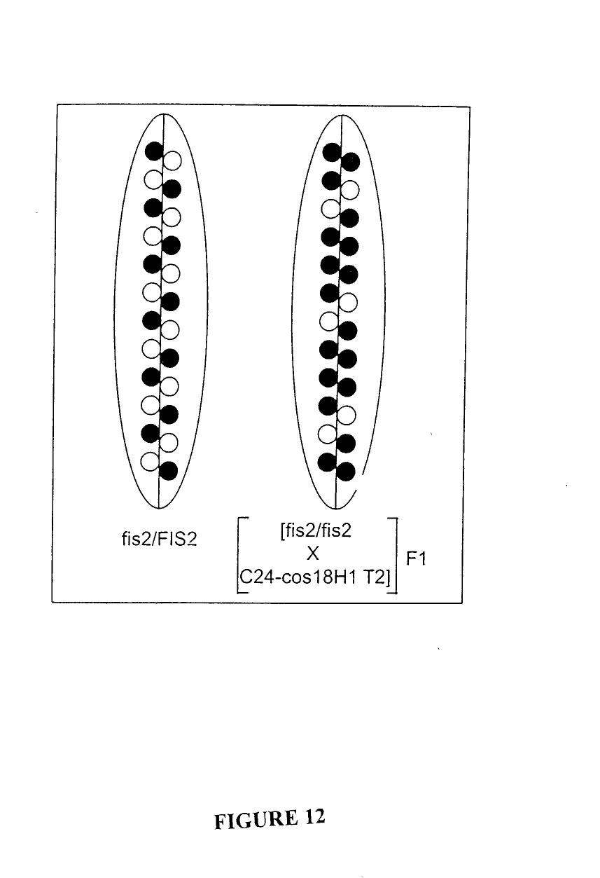

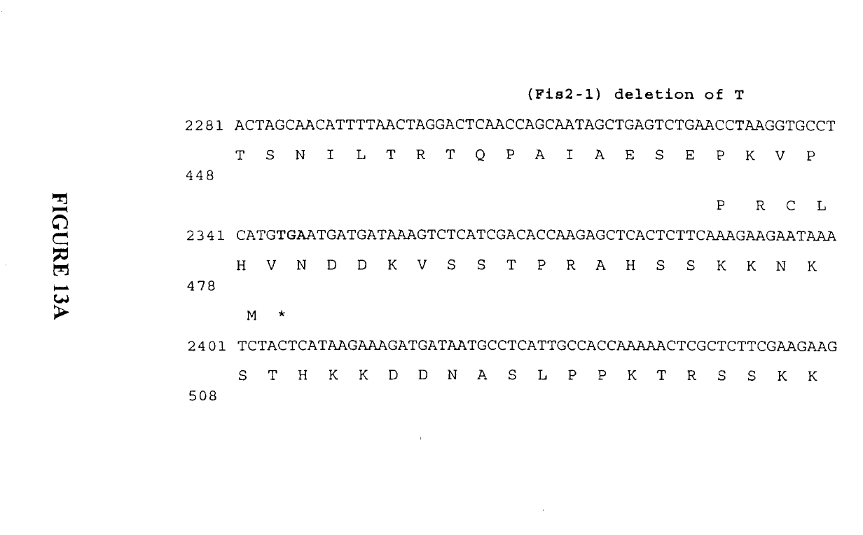

The ratios of arrested seeds were scored. The ratio of fis:FIS seeds was predicted to shift from the 1:1 ratio expected in the absence of complementation, to a ratio of 1:3 expected following complementation. In the seed of six independent kanamycin-resistant F1 lines, a segregation ratio of 3:1 (FIS:fis) was in fact observed (FIG. 12). In contrast, the same ratio shift was not observed in kanamycin-sensitive plants of the same cross.

These data indicate that the cosmid clone pOCA18H1 complements the fis2 mutant phenotype and contains the FIS2 gene.

### EXAMPLE 8

### Isolation of the FIS2 cDNA Clone

DNA probes derived from the EcoRI fragments E1 and E2 were used to screen 200,000 plaques from an Arabidopsis late silique cDNA library obtained from Anna Koltunow (CSIRO, Div. of Plant Industry, Adelaide, Australia). Prehybridisation and hybridisation were performed in 10% PEG₆₀₀₀, 7% (w/v) SDS, 0.25 M NaCl, 0.05 M NaPO₄ at pH 7.2, 1% (w/v) bovine serum albumin, 1 mM EDTA at 65° C. for 2 hrs and 16 hr, respectively. The filters were washed at room temperature (once in 2×SSC, 1% SDS for 30 min each) and exposed O/N on X-ray film with 2 intensifying screens at −70° C.

A total of 4 positive cDNA clones were obtained, two of which hybridised to DNA probe derived from the left hand side of the DSG insertion and the two others hybridised to DNA probe derived from the left hand side of the DSG insertion. These 4 plaques were purified, excised, analysed by restriction mapping and sequenced.

The DNA isolated from positive plaques of the Arabidopsis late silique cDNA library from were sub-cloned in vivo from the LambdaZap [symbol] vector using the ExAssist [symbol] interference resistant helper phage.

Sequencing was performed by double-stranded sequence analysis on an Applied Biosystems Model 370A DNA Sequencer using a fluorescent dye-labelled dideoxy terminator kit. The sequence data were analysed using computer software DNA Strider for MacIntosh (Marck, 1988), and the GCG Sequence Analysis Package software (Devereux, 1984).

The nucleotide sequence of the full-length FIS2 cDNA clone is presented in SEQ ID NO:6. The derived amino acid sequence of this cDNA clone is presented in SEQ ID NO:2.

The cDNA inserts which hybridised to the right hand side of the DSG insertion in the transposon-tagged line had the same 3′-end sequence, indicating that they both came from the same gene and that the longest cDNA clone was potentially full length. The longest cDNA was designated CTF1. The 5′-end of CTF1 was about 750 bp to the right of the DSG insertion. Almost 400 bp at the 3′-end of CTF1 were not on the E2 fragment (FIG. 11) but on the adjacent EcoRI fragment, designated E4 in FIG. 11.

Those cDNA inserts which hybridised to the left hand side of the DSG insertion were both about 1.7 kb long. One clone, designated CTF2a, shared 100% nucleotide sequence identity with the genomic sequence of the E1 fragment (FIG. 11). The second clone, designated CTF2b, shared 85% nucleotide sequence identity with CTF2a, indicating that CTF2a and CTF2b contained related cDNAs which are variants of the same gene family. CTF2a is in the same orientation as CTF1, indicating that the 3′-end of CTF2a was located 500 bp from the junction between the EcoRI fragments E1 and E2 and, as a consequence, more than 2 kb from the DSG insertion.

### EXAMPLE 9

### Construction and Screening of a Genomic Library to Isolate the fis2-2 Gene

Genomic DNA from the DSG-tagged mutant fis2-2 was digested using the enzyme Sau3AI and size-fractionated on a glycerol gradient. The 10-12 kb fraction was then ligated into bacteriophage EMBL4 BamHI-digested and dephosphorylated arms. The ligated DNA was packaged into sonicated extract BHB2690 and freeze-thaw lysate from induced packaging proteins BHB2688. The number of plaque-forming units (PFU) of the recombinant bacteriophage was determined by plating the bacteriophage onto solid media plates using Escherichia coli strain K803 cells. Approximately 9×10⁴ PFU were transferred from plates onto nylon filter membranes and screened using a BamHI fragment containing the 5′-end of DSG and E2 as probes. Prehybridization and hybridization were performed at 42° C. for 45 min and overnight, respectively, in a solution comprising 50% (v/v) formamide, 3×SSC, 21.5× Denhardt [symbol] s Solution, 0.1% (w/v) SDS and 0.5 mg/ml salmon sperm DNA. The filters were washed at room temperature twice in 2×SSC, 0.1% (w/v) SDS for 15 min each wash and twice in 0.1×SSC, 0.1% (w/v) SDS for 15 min each wash, before exposing the filters to X-ray film with an intensifying screen at −80° C.

Positive-hybridizing plaques were plaque-purified in subsequent screening rounds and sequenced as described in Example 8.

The nucleotide sequence of the wild-type FIS2 gene is presented herein as SEQ ID NO:7.

Nucleotide sequence analysis of the 5′-region of the FIS2 gene sequence was performed, using www.NETGENE2, to predict intron-exon splice junctions. Data obtained from the WWW.NETGENE2 server in relation to the confidence of the predicted splice sites in the FIS2 gene are presented in Table 3.

TABLE 3
Confidence for the predicted intron splice
sites of the FIS2 gene

| | | Confi- | SEQ | |
|---|---|---|---|---|
| Posi- | Acceptor/ | dence | ID | |
| tion | Donor | Level ¹ | NO: | Nucleotide Sequence* |
| 590 | Donor | 1.00 | 200 | AAAAAACAAC gtatgcattc |
| 875 | Acceptor | 0.56 | 201 | gtttattcag CCATATTTCC |
| 932 | Donor | 0.88 | 202 | CTACAGGGAT gtgagtaaca |
| 1228 | Acceptor | 0.86 | 203 | ttttgcttag GTCAAATTCA |
| 1300 | Donor | 1.00 | 204 | AAAGCTGAAG gtgagccttt |
| 1401 | Acceptor | nd* | 205 | ccaaatgcag TAGTGGAAAA |
| 1454 | Donor | 0.94 | 206 | AGGTCACGAG gtaggcacta |
| 1582 | Acceptor | nd | 207 | ttgtgccacag GGCTTGCAAC |

*,Intron sequences are shown in lower case and exons in upper case.
nd, not determined.
¹, The cutoff value for each confidence level is as follows:
Highly confident donor sites: 95%
Highly confident acceptor sites: 95%
Nearly all true donor sites: 50%
Nearly all true acceptor sites: 20%

The present inventors have further analysed the genomic structure of the FIS2 gene present in Arabidopsis thaliana ecotype Columbia. Compared to the nucleotide sequence of the FIS2 gene present in the Landsberg ecotype, a 180 bp deletion occurs in exon 8 of the Columbia ecotype, producing a 60 amino acid deletion in the derived amino acid sequence of the FIS2 polypeptide encoded therefor. PCR analysis of the same region in the Arabidopsis thaliana ecotypes C24 and WS indicated that the deletion was ecotype-specific and only present in the Columbia ecotype.

Additionally, the FIS2 gene of Arabidopsis thaliana ecotype Columbia comprises a 26 bp deletion in intron 7 compared to Arabidopsis thaliana ecotype Landsberg.

### EXAMPLE 10

### The fis2 Mutant Phenotype Results from Single Basepair Changes

In order to determine the nucleotide sequence the fis2 mutant gene, seven amplification primer pairs were designed, based upon the nucleotide sequence of the CTF1 cDNA clone. These primers were synthesized using an Applied Biosystems automatic DNA synthesizer Model 394.

The primer pairs were used to amplify and sequence the mutant fis2 gene from genomic DNA derived from fis2-1, fis2-2, and fis2-3 homozygous mutant plants. Each primer pair amplified a 500-600 base pair fragment from genomic DNA.

PCR was carried out in 20 ml of 50 mM KC1, 10 mM Tris-HC1 pH 9.0, 0.1% (v/v) Triton X-100, 2 mM of each primer, 0.4 mM dNTP, 1.5 mM MgC1₂, and 2 units/reaction TaqI DNA polymerase. The PCR conditions comprised a first denaturation step of 5 min duration at 94° C., followed by thirty cycles, each cycle comprising:

(i) denaturation at 94° C. for 20 sec;

(ii) annealing at 55° C. for 30 sec:

(iii) polymerisation at 72° C. for 30 sec; and

a final incubation at 25° C. for 1 min. Reactions were performed using a Corbett Research Capillary Thermal Sequencer Model FTS-1S.

PCR products were purified using Wizard Prep and sequenced directly. If necessary, PCR products were purified from 1% (w/v) agarose gels following electrophoresis thereon, prior to being sequenced.

Sequencing reactions were carried out as described in Example 8.

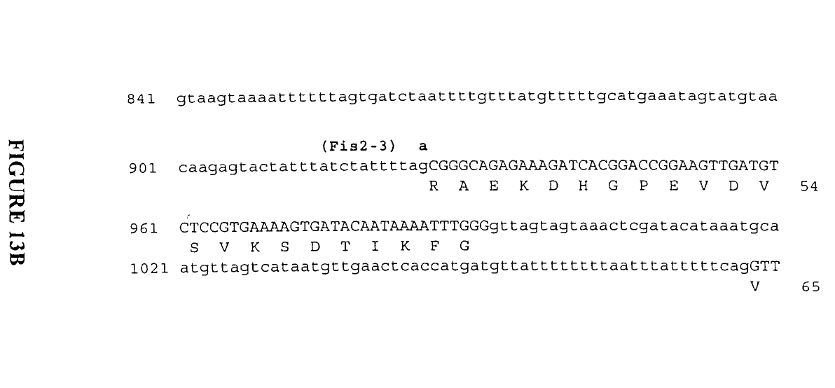

The nucleotide sequence of the fis2-1 mutant allele revealed a 1 bp deletion in exon 8, in the region corresponding to position 1835 in the wild-type FIS2 cDNA (SEQ ID NO:6). This mutation produced a frame-shift in the mutant fis2-1 allele compared to the wild-type allele, thereby terminating translation of the FIS2-1 polypeptide four amino acids downstream of the deletion point (FIG. 13A).

The nucleotide sequence of the fis2-3 mutant allele revealed a single base change at the 3′-splice junction of intron 5, producing the mutation of AG to AA (FIG. 13B). Similar single base changes in intron splice junctions have been reported for other EMS-induced mutants (Sun and Kamiya, 1994).

### EXAMPLE 11

### The FIS2 Polypeptide is a Putative Transcription Factor

The derived amino acid sequence of the FIS2 polypeptide is presented herein as SEQ ID NO:2. In this regard, there are three in-frame putative translation start sites in the FIS2 cDNA, commencing at nucleotide positions 1 and 37 and 364 of SEQ ID NO:SEQ ID NO:6.

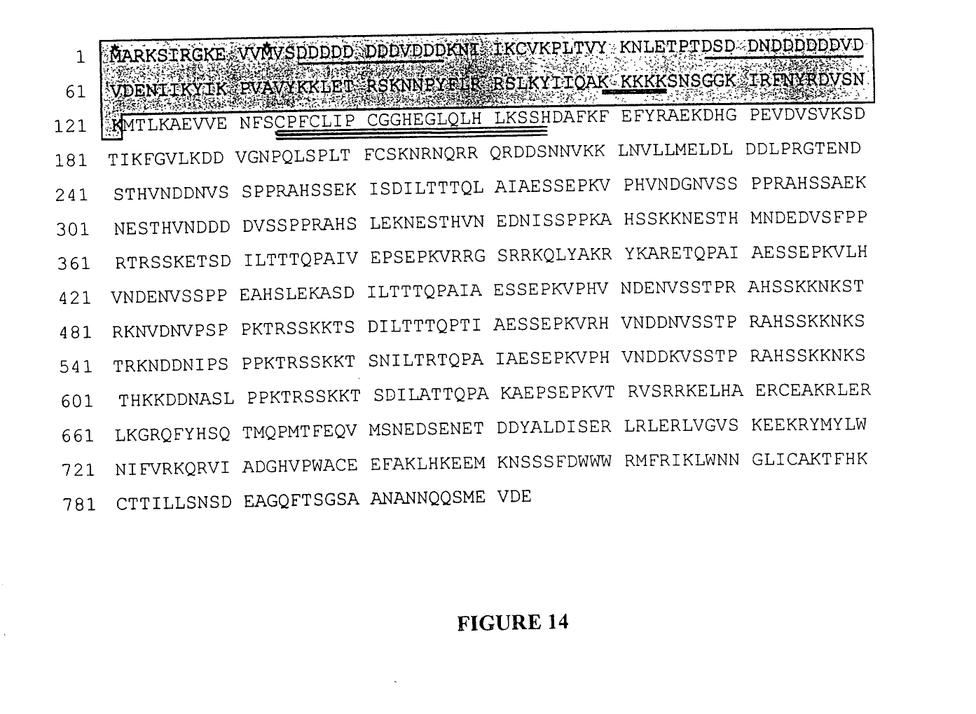

A search for known protein motifs in derived amino acid sequence of the FIS2 polypeptide revealed a putative C2H2 zinc-finger motif within the first 151 residues of the polypeptide, and several putative nuclear localization signals (NLS) distributed between residues 1 to 661 of the FIS2 protein (FIG. 14). However, as stated in Example 15 below, in vivo expression data suggest that the true NLS is localised within the first 121 amino acids of the FIS2 polypeptide (shaded region in FIG. 14).

Amino acid sequences which contain zinc finger motifs are generally nucleic acid binding proteins in which the finger structures are maintained by the cysteine and/or histidine residues of the C2H2 zinc-finger motif being organized around a zinc metal ion (Stanojevic et al., 1989; Berg, 1993). Several members of the C2H2 zinc-finger proteins, also known as the TFIIIA/Kruppel-like zinc-finger protein gene family, play important and diverse roles in growth and development in Drosophila melanogaster (Stanojevic et al, 1989; Treisman and Desplan, 1989). Recently, C2H2 zinc-finger proteins have been identified in plants (Meissner and Michael, 1997; Takatsuji, et al., 1994); Takatsuji et al, 1991; Sakai et al, 1995; Tague and Goodman, 1995).

The presence of both the zinc finger motif and the NLS suggests that the FIS2 polypeptide may well be a transcription factor belonging to the TFIIIA or Kruppel-like zinc-finger protein gene family.

Another characteristic of the FIS2 polypeptide is a high content of serine residues (12.9%), a characteristic feature of other C2H2 zinc-finger proteins (Tague and Goodman, 1995).

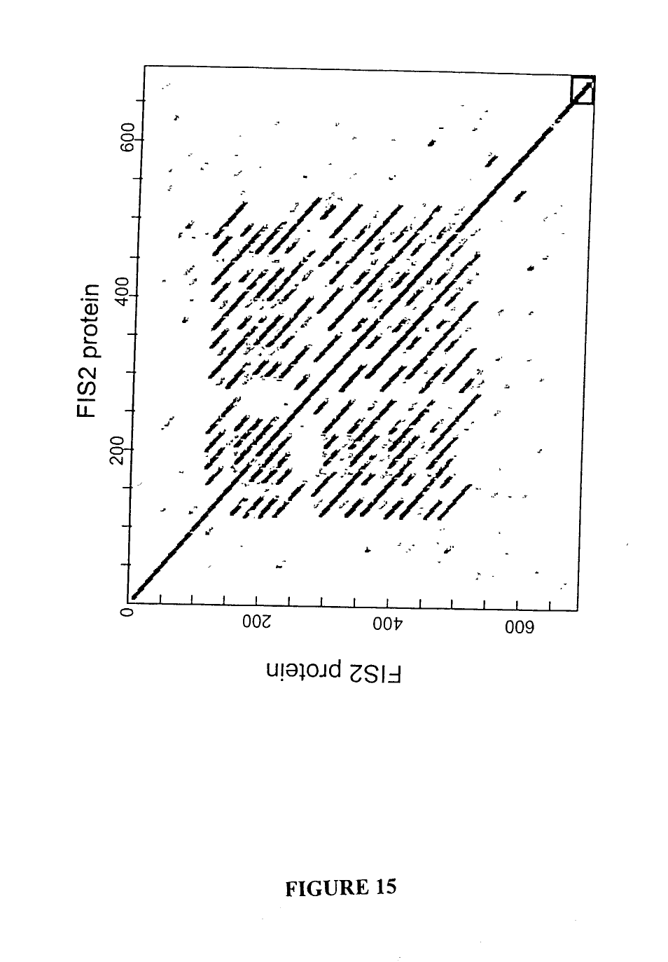

Additionally, the FIS2 polypeptide comprises highly repetitive amino acid sequences, located between residues 243 and 642 of SEQ ID NO:2 (FIG. 14). The repeat comprises a core of 22 amino acid residues in length, which is repeated 12 times Although the core sequence is not 100% identical among the 12 repeats, the homology is easily detectable using sequence analysis and dot matrix computer program (FIG. 15). The repeated region is likely to be involved in protein-protein interactions, suggesting that the FIS2 polypeptide may be one component of a protein complex.

### EXAMPLE 12

### The FIS2 Gene is a Single Copy Gene

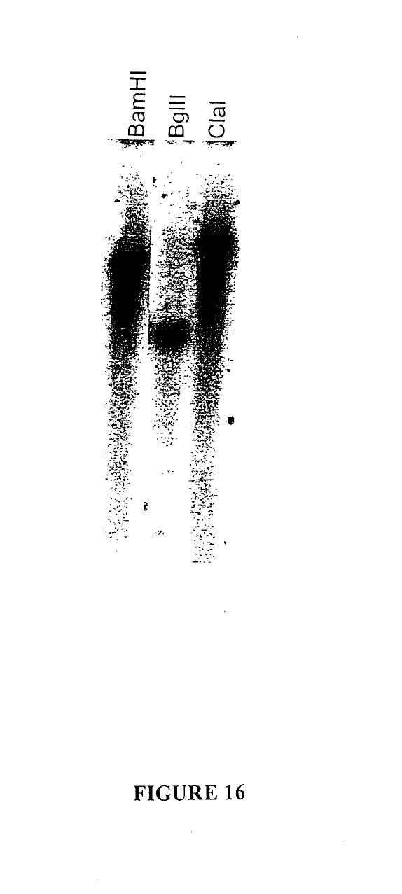

Genomic DNA from Arabidopsis seedlings was prepared by the CTAB protocol (Taylor, 1982; Dellaporta, 1983). Genomic DNA (5 μg) was digested with restriction enzymes prior to electrophoresis on 1% (w/v) agarose gels. The DNA was then transferred to a HybondN membrane, prehybridized for 1 hr, hybridized and the filters were washed according to Church and Gilbert (1984). Probes were labelled with [−32P]-dCTP using the random primer method (Feinberg and Vogelstein, 1983). This analysis revealed that the FIS2 gene is a single copy gene (FIG. 16).

### EXAMPLE 13

### Expression of the FIS2 Gene in Plants

Total RNA was prepared individually from Arabidopsis thaliana roots, shoots, leaves, stems, and flowers according to Dolferus (1994). Total RNA was also prepared from siliques using the phenol extraction method.

Total RNAs were DNase-treated and RT-PCR (McPherson, 1991) was performed on 2 mg of RNA using the primers 1F (SEQ ID NO:208: 5′TCATCTCTTCCTTATGMGTT-3′) and 2R (SEQ ID NO:209: 5′-TGTTGATAATGTCCCATCG-3′) which anneal in the region of exon 12 and exon 8, respectively. First strand cDNA was synthesized for 1 hr at 37° C. in 50 mM Tris-HC1 at pH8.3, 10 mM MgCl₂, 75 mM KC1, 10 mM DTT, 0.5 mM dNTP, 4 units RNasin (Promega) and 5 units MMLV reverse transcriptase (Epicentre). PCR amplification was then carried out on 5 I of RT reaction in a final volume of 20 l, containing 50 mM KC1, 10 mM Tris-HC1 pH 9, 0.1% (v/v) Triton X-100, 1 mM of each primer, 0.4 mM dNTP, 1.5 mM MgC1₂ and 2 units of TaqI DNA polymerase (Perkin-Elmer). The amplification reaction comprised a first denaturation step of 5 min duration at 94° C., followed by thirty cycles, each cycle comprising:

(i) a 20 sec denaturation step performed at 94° C.;

(ii) a 20 sec annealing step performed at 55° C.; and

(iii) a 1 min elongation step performed at 72° C.,

followed by a final cycle comprising incubation for 2 min at 72° C., followed by 1 min at 28° C. Amplification reactions were performed using a Corbett Research Capillary Thermal Sequencer Model FTS-1S. RT-PCR products were separated by agarose gel (1%) electrophoresis.

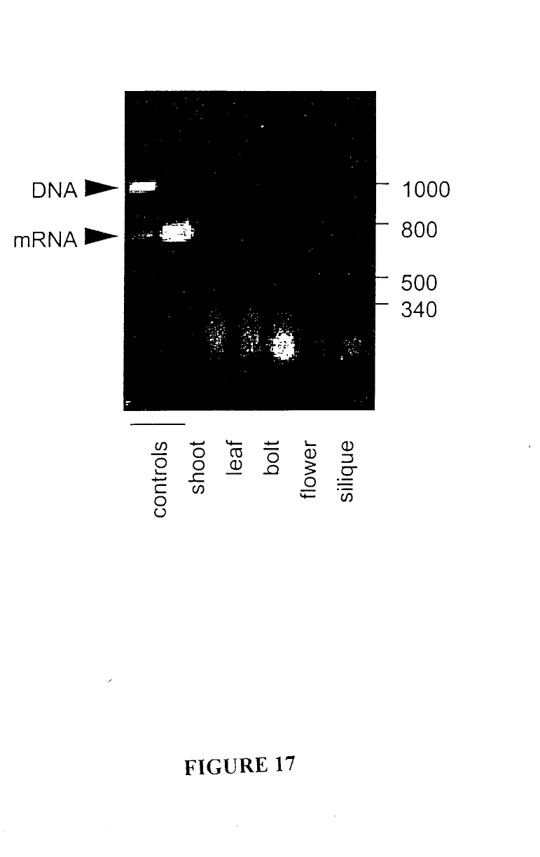

Amplification products corresponding to the FIS2 transcript were present at least in shoots, leaves, bolts and siliques, with a much weaker signal present in flowers (FIG. 17).

### EXAMPLE 14

### Nucleotide Sequence of the FIS1 Gene and Structure of the FIS1 Polypeptide

The nucleotide sequence of the cDNA encoding the FIS1 polypeptide is presented in SEQ ID NO:4.

Genomic clones encoding the FIS1 polypeptide were obtained and nucleotide sequences were obtained as described herein. The nucleotide sequence of the FIS1 gene is presented in SEQ ID NO:5.

The fis1 mutation maps to the same locus as the mea mutation. Accordingly, the amino acid sequence of the FIS1 polypeptide set forth in SEQ ID NO:1 corresponds to the sequence disclosed by Grossniklaus et al. (1998).

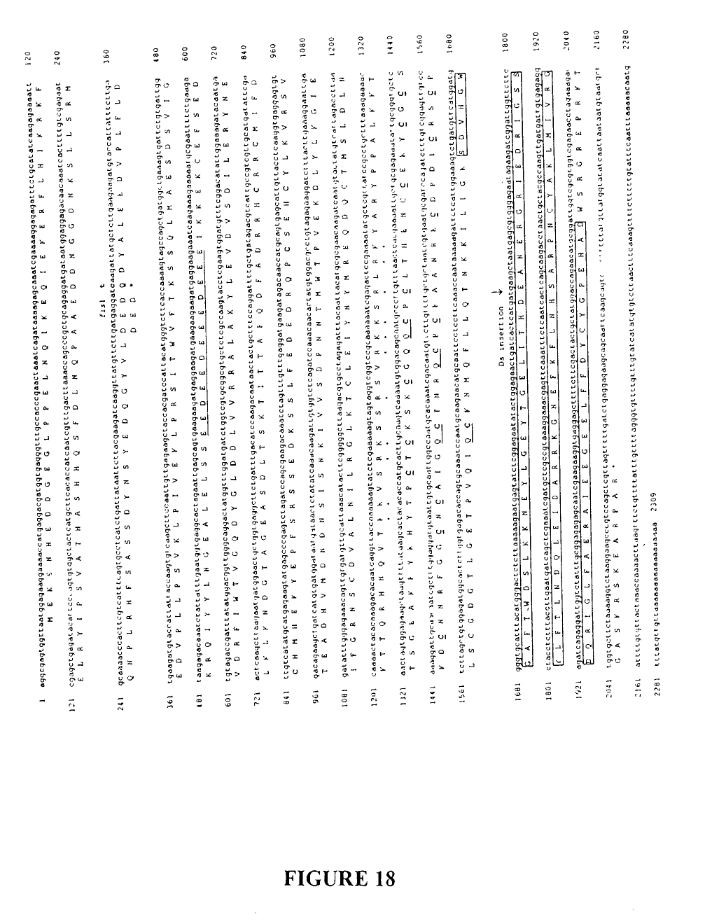

DNA derived from the fis1 homozygous mutant was sequenced using MEA gene primers and a single base change was found in fis1 mutant compared to the wild-type MEA gene sequence disclosed by Grossniklaus et al (1998). This single base change introduced a translation stop codon in the 5′-region of the open reading frame of the MEA gene, thereby resulting in early termination of translation and the synthesis of a truncated polypeptide (FIG. 18). Accordingly, the fis1 allele is a presumptive null allele. In particular, the single base change comprised the substitution of a thymidine residue for a cytidine residue at position 320 of SEQ ID NO:4, producing a stop codon TAA in this region which results in translation being terminated at amino acid 102 in SEQ ID NO:1 of the FIS1 polypeptide.

In contrast, the mea mutation comprises a Ds transposon inserted into the C-terminal region of the gene, in particular at the junction between nucleotide positions 1756 and 1757 in SEQ ID NO:4. Accordingly, in the medea mutation the insertion is such that a polypeptide with a short truncation in the carboxyl terminal results.

The fis1 mutant gene is an allele of the MEA gene. The different phenotype of the fis1 mutant compared to the mea mutant, indicates that the point mutation in fis1 is critical to reduce expression of the wild-type MEA/FIS1 gene to a biologically inactive level which is sufficient to facilitate autonomous seed development.

The MEDEA/FIS1 polypeptide (SEQ ID NO:1) comprises at least the following peptide motifs or protein domains:

(i) an acidic domain, presumably required for interaction with other polypeptides;

(ii) a C5 motif comprising five conserved cysteine residues and having an unknown function;

(iii) a putative nuclear localization signal;

(iv)a CXC domain comprising a stretch of cysteine residues, of unknown function; and

(v) a SET domain, which is shared by some of the polycomb group of proteins, including E(z) (i.e. enhancer of zeste).

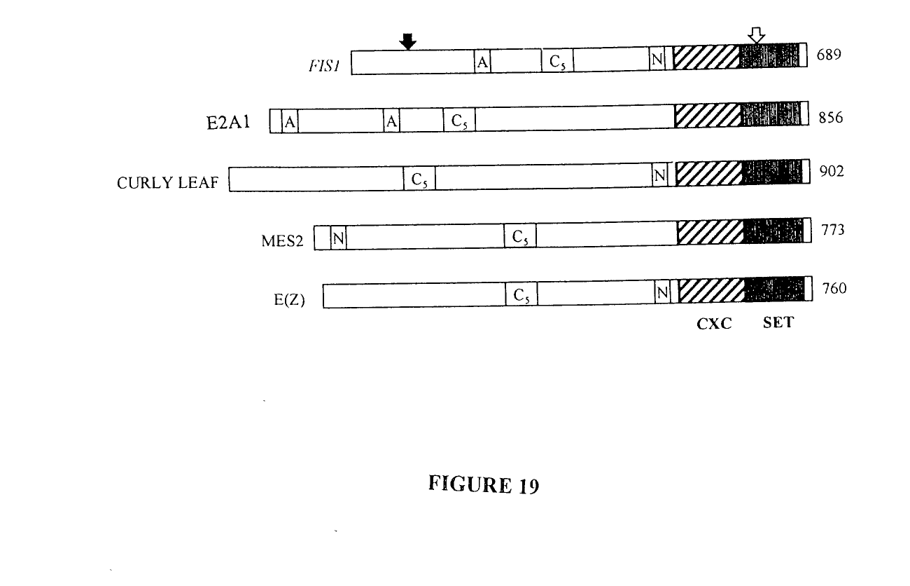

The Arabidopsis thaliana Polycomb group proteins designated EZA1 and CURLY LEAF and the Drosophila melanogaster E(z)polypeptide and the Caenorhabditis elegans MES-2 polypeptide also comprise the SET domain, the CXC domain, C5 domain and a nuclear localisation signal (FIG. 19).

Comparison of the fis1 and mea alleles indicates that in the fis1 mutant, none of these five structural motifs are present, whilst in the mea mutant all domains except the SET domain are present. The phenotypic difference between fis1 mutant and mea suggests that the structural motifs present in the MEDEA/FIS1 polypeptide may be biologically significant in regulating fertilization independent seed development in plants, whilst the SET domain alone may be important in embryogenesis.

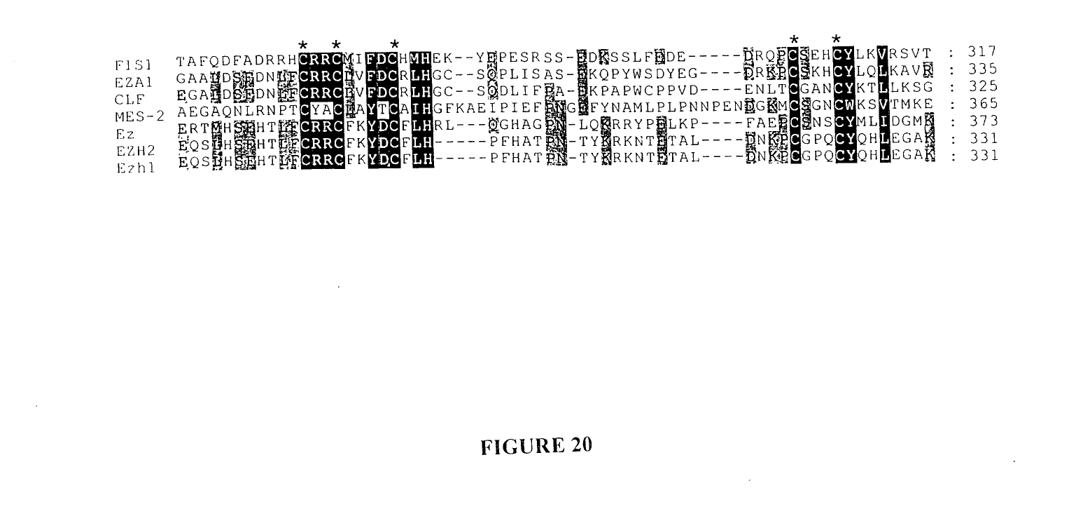

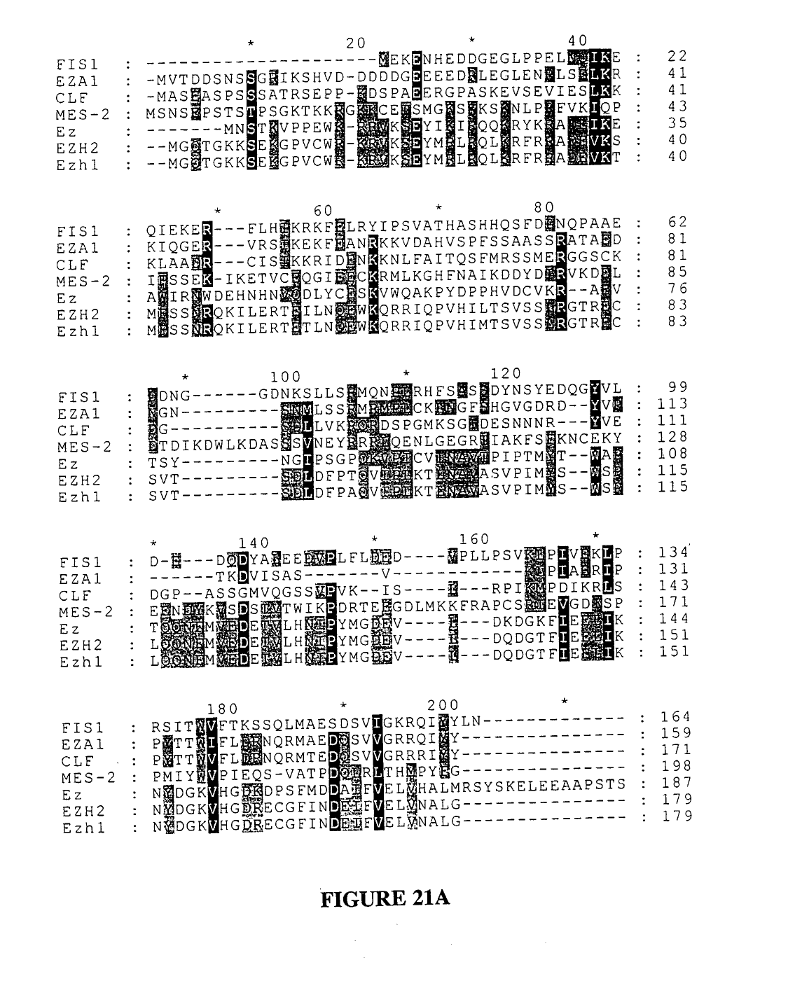

Sequence alignment of various E(z)-like proteins around the C5 cysteine-rich domain using program ClustalW (Thompson et al., 1994; FIG. 20) revealed the following consensus sequence, as represented by the amino acid sequences contained in any one or more of SEQ ID NO:10 to SEQ ID NO:55:

-R-R-C-X₂-[F/Y]-D-C-X-[M/L]-H-X₍₂₂₋₃₂₎-C-X₃-C-Y,

wherein numerical values indicate the number of consecutive amino acids in the consensus sequence.

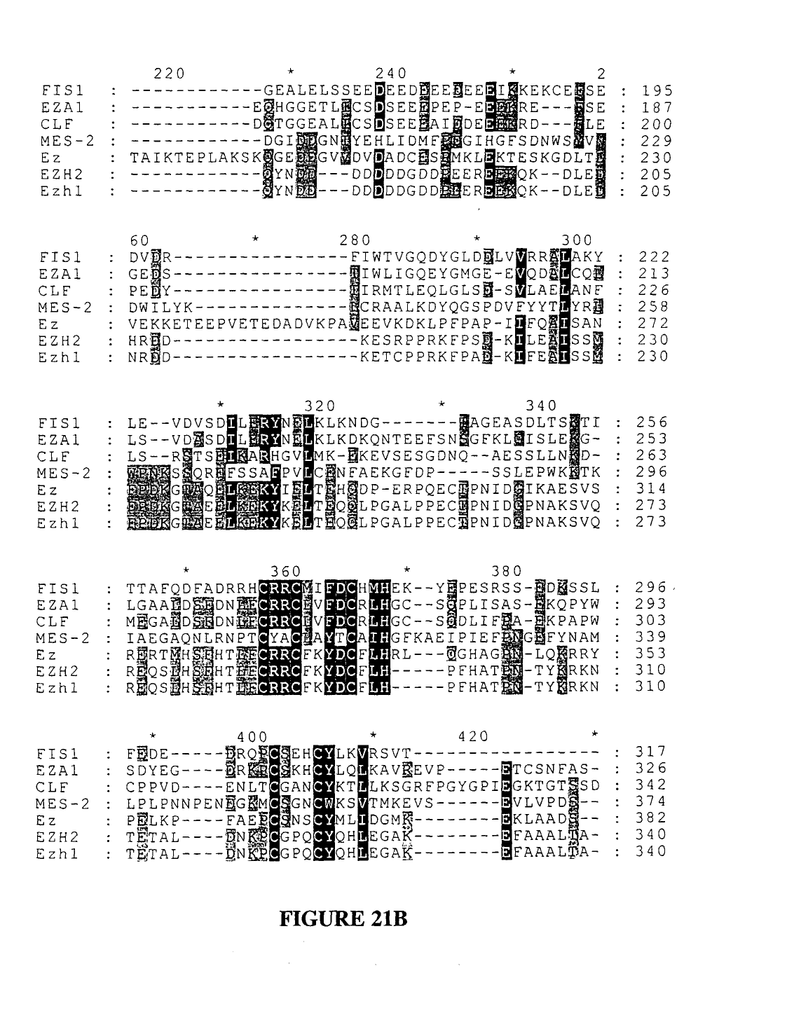

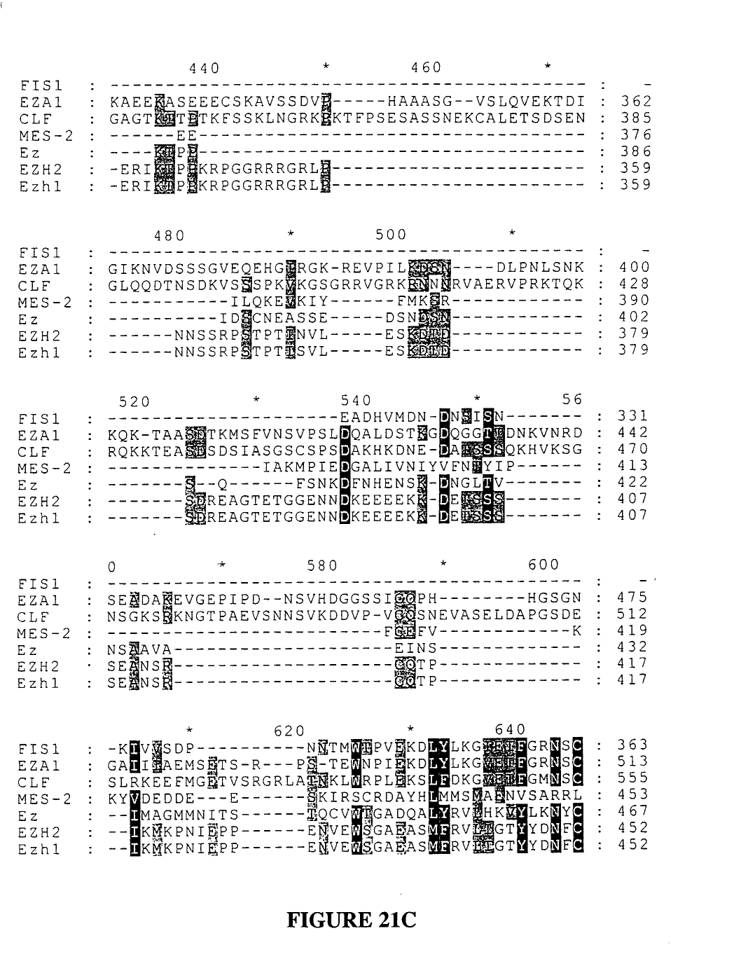

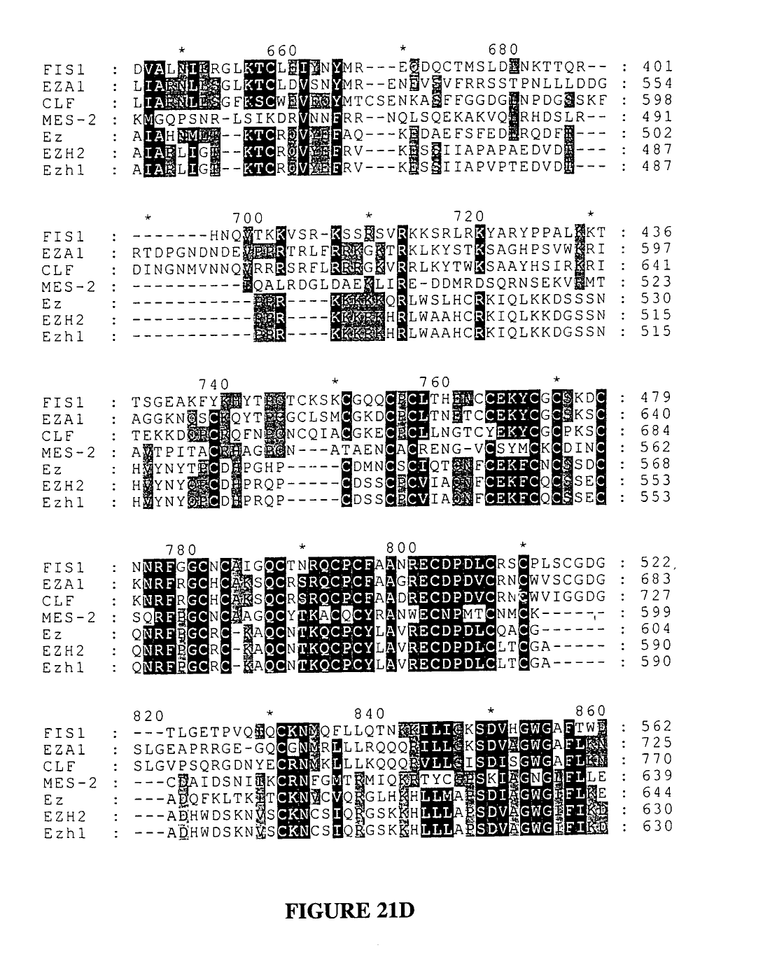

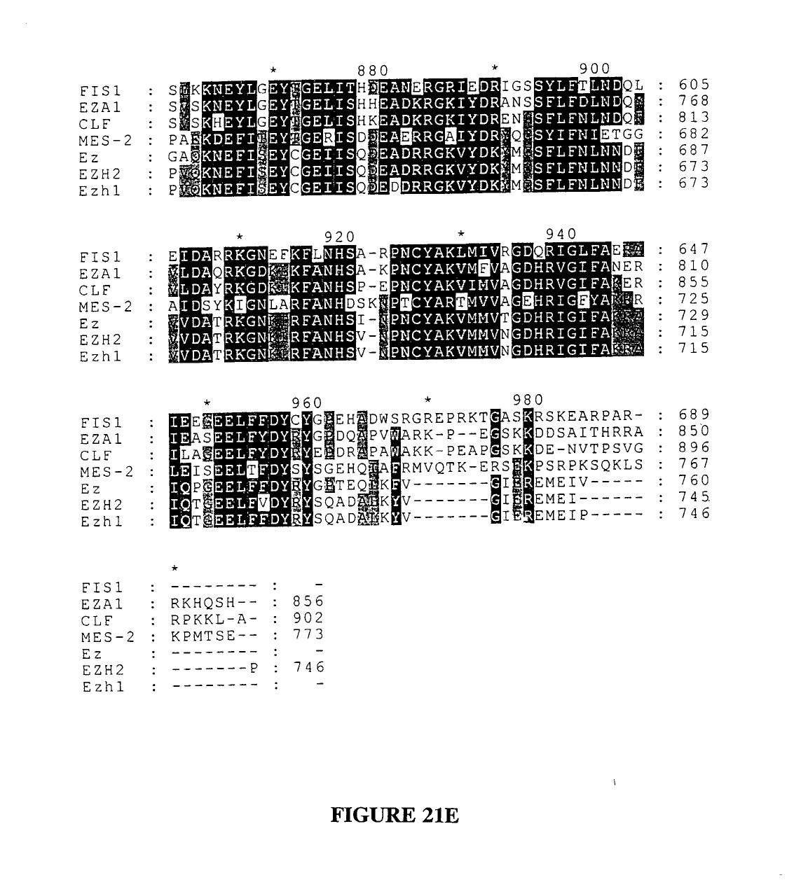

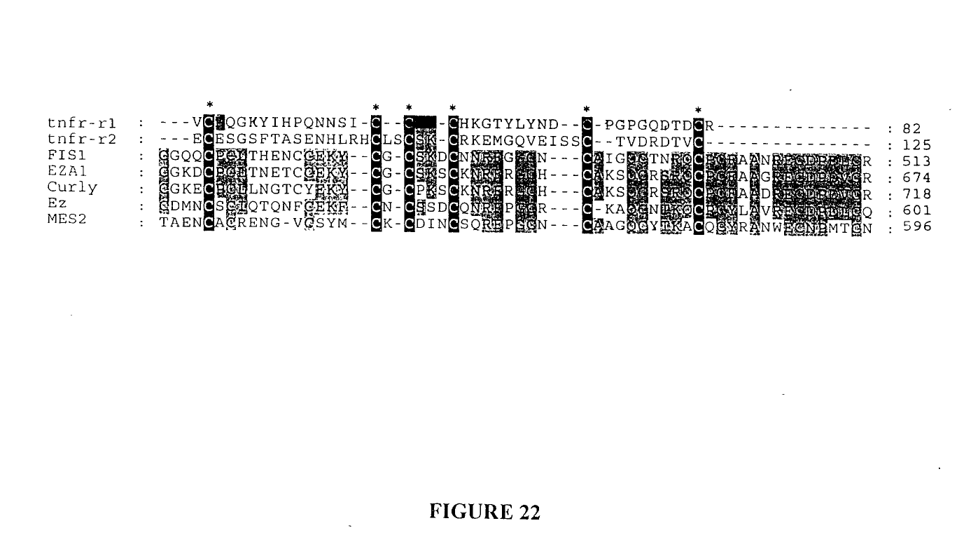

Additional motifs have been identified within the E(z) class of polypeptides, including the FIS1 polypeptide, by aligning the amino acid sequence of MEDEA/FIS1 to the amino acid sequences of several E(z) polypeptides, using the multiple sequence alignment program ClustalW (Thompson et al., 1994). The aligned amino acid sequences of MEDEA/FIS1, EZA1, CURLY LEAF, E(z) and MES-2 are presented in FIGS. 21A-21E.

This analysis revealed strong homology in the SET domain, CXC domain, C5 domain, in addition to a putative TNFR/NGFR motif (FIG. 22) and an RGD motif which had not been previously identified for this class of proteins.

The TNFR/NGFR domain overlaps the previously-described CXC domain in MEDEA and other E(z)-like proteins. This consensus domain consists of about 40 amino acids, containing 6 conserved cysteine residues. The TNFR/NGFR domain is defined by a general consensus sequence as represented by any one or more of the amino acid sequences set forth in SEQ ID NO:116 to SEQ ID NO:180, as follows:

C-X_{(4,6)}-[F/Y/H]-X_{(5,10)}-C-X_{(0,2)}-C-X_{(2,3)}-C-X_{(7,11)}-C-X_{(4,6)}-[D/N/E/Q/S/K/P]-X₂-C,

wherein numerical values indicate the number of consecutive amino acids in the consensus sequence. The motif may be found from 1 to 4 times in a given protein sequence. TNFR family members regulate processes that range from cell proliferation to programmed cell death. This domain is also found in cytokine receptor (CD40, CD27, CD30), in FAS antigen, the receptor for FASL, a protein involved in apoptosis, and other cytokine receptor proteins. The TNFR/NGFR motif is also present in the proteins designated TNFR-R1 and TNFR-R2 (FIG. 22).

Of all the E(z) proteins analysed, only the MEDEA/FIS1 polypeptide comprised a close match to the TNFR/NGFR motif found in the MOTIF database at 100%. The other E(z)-like proteins shown in FIG. 22 do not match this amino acid sequence motif at 100% using the MOTIF program. Although the CXC domain found in all the E(z)-like sequences contains the 6 conserved cysteine of the TNFR/NGFR domain with the correct spacing between each of them, at least one of the other conserved residues is different in these other protein sequences.

The sequence Arg-Gly-Asp (SEQ ID NO:181) which is present in the MEDEA/FIS1 polypeptide, is also found in fibronectin where it is crucial for its interaction with its cell surface receptor, an integrin Ruoslahti and Piersbacher (1986). The motif is also found in other proteins (e.g. collagen, vitronectin, fibrinogen and snake disintegrin), where it has been shown to play a role in cell adhesion. The role of this motif in the FIS1 polypeptide in unclear.

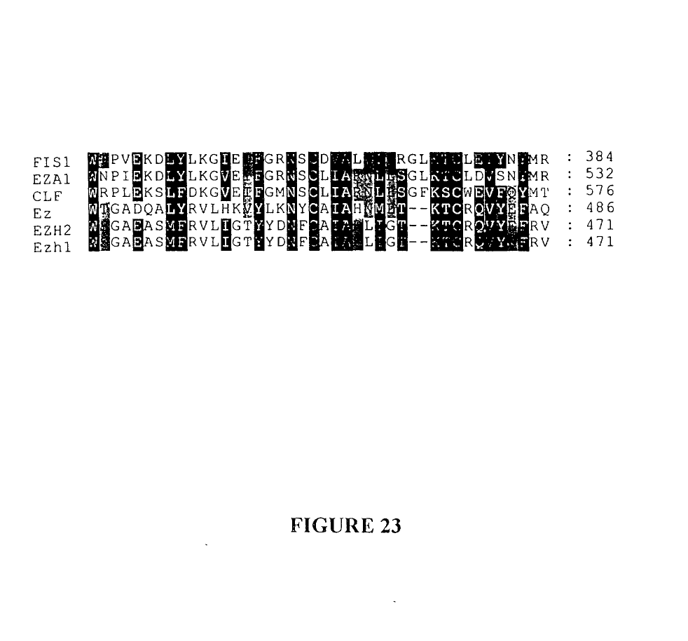

A further novel motif was identified C-terminal to the C5 domain and N-terminal to the CXC domain in the MEDEA/FIS1 polypeptide, designated as the WCA motif (FIG. 23), which comprises the amino acid sequence set forth in SEQ ID NO:189:

W-T-P-V-E-K-D-L-Y-L-K-G-I-E-I-F-G-R-N-S-C-D-V-A-L-N-I-L-R-G-L-K-T-C.

Alignment of the E(z) polypeptide to the E(z)-like polypeptides MEDEA/FIS1, CURLY, EZA1 and MES-2 reveals the consensus sequence as respresented by the amino acid sequence set forth in SEQ ID NO:185, as follows:

W-X-(P/R/G)-X-(E-A-D)-X₂-(L/M)-(Y/F/M)-X-(K/S/V)-(G/M/L)-X-(E/K/G)-I-F-G-X-N-S-C-X-(I/V)-A-X-(N/H)-(L/I/M)-(L/M)-X-G-X-K-(T/S)-C,

or alternatively, the consensus sequence as respresented by the amino acid sequence set forth in SEQ ID NO:186, as follows:

W-X-(P/G)-X-(E/D)-X₂-(L/M)-(Y/F)-X-(K/V)-(G/L)-X₃-(F/Y)-(G/L)-X-N-X-C-X-(I/V)-A-X-(N/L)-(L/I/M)-(L/G)-X₁₋₃-K-(T/S)-C.

### EXAMPLE 15

### FIS1 and FIS2 Promoter GUS Fusions Show Similar Pattern of Expression

We studied the expression pattern of the FIS1 and FIS2 genes, by fusing their promoter sequences to the GUS reporter gene, introducing the FIS promoter/GUS fusion constructs into plant cells, regenerating whole plants therefrom and determining the GUS staining pattern in the transgenic plants.

In particular, two different the FIS1 promoter/GUS fusion constructs were produced as follows, and introduced into A. thaliana using standard procedures for the transformation of this plant species:

(i) A 1357 bp FIS1 promoter GUS construct, including nucleotides from 440 bp upstream of the translation initiation site of the FIS1 gene, to about 917 bp downstream of the translation initiation site of the FIS1 gene (i.e. about nucleotides 1785 to 3143 of SEQ ID NO:5); and

(ii) a 2987 bp FIS1 promoter GUS construct, including nucleotides from 2070 bp upstream of the translation initiation site of the FIS1 gene, to about 917 bp downstream of the translation initiation site of the FIS1 gene (i.e. about nucleotides 156 to 3143 of SEQ ID NO:5).

Each FIS1/GUS fusion construct contained the complete sequence of exons 1 and 2, and 80 bp of exon 3, including the first 2 introns of the FIS1 gene nucleotide sequence (SEQ ID NO:5).

Two different the FIS2 promoter/GUS fusion constructs were also produced as follows, and introduced into A. thaliana using standard procedures for the transformation of this plant species:

(i) A 1620 bp FIS2 promoter GUS construct, including nucleotides from 1281 bp upstream of the translation initiation site of the FIS2 gene, to about 339 bp downstream of the translation initiation site of the FIS2 gene (i.e. about nucleotides 1908 to about nucleotides 3528 of SEQ ID NO:7); and

(ii) a 3528 bp FIS2 promoter GUS construct, including nucleotides from 3189 bp upstream of the translation initiation site of the FIS1 gene, to about 339 bp downstream of the translation initiation site of the FIS1 gene (i.e. about nucleotides 1 to 3528 of SEQ ID NO:7).

Each FIS2/GUS fusion construct contained the complete sequence of exons 1, 2 and 3, and 39 bp of exon 4, including the first 3 introns of the FIS2 gene nucleotide sequence (SEQ ID NO:7). The putative zinc-finger protein motif found in the FIS2 polypeptide was also included the FIS2/GUS fusion protein products of these two FIS2/GUS fusion constructs.

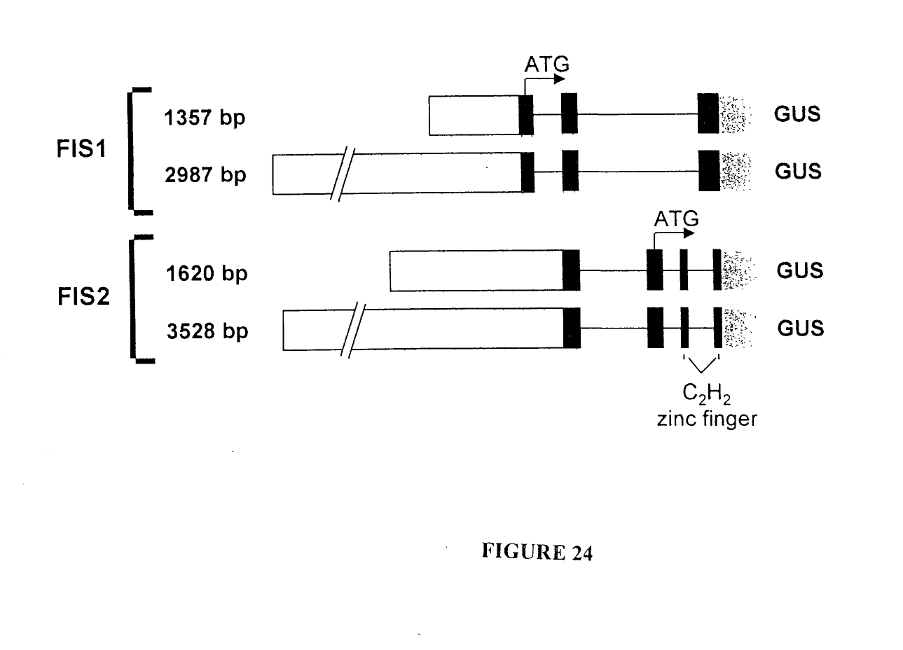

The FIS1/GUS and FIS2/GUS fusion constructs described herein are represented schematically in FIG. 24.

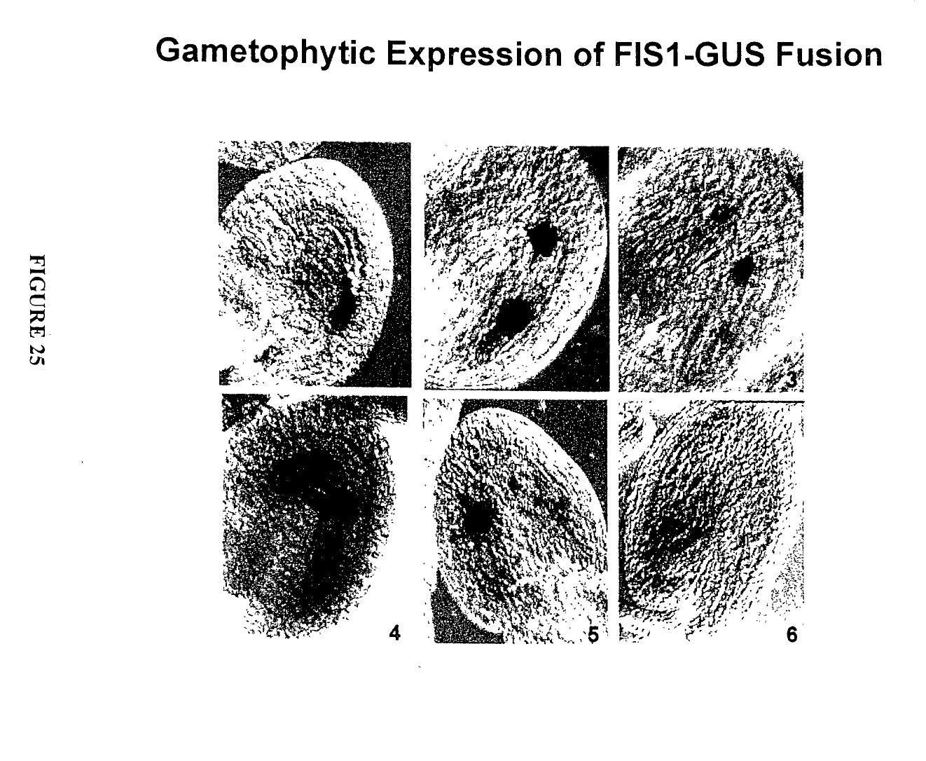

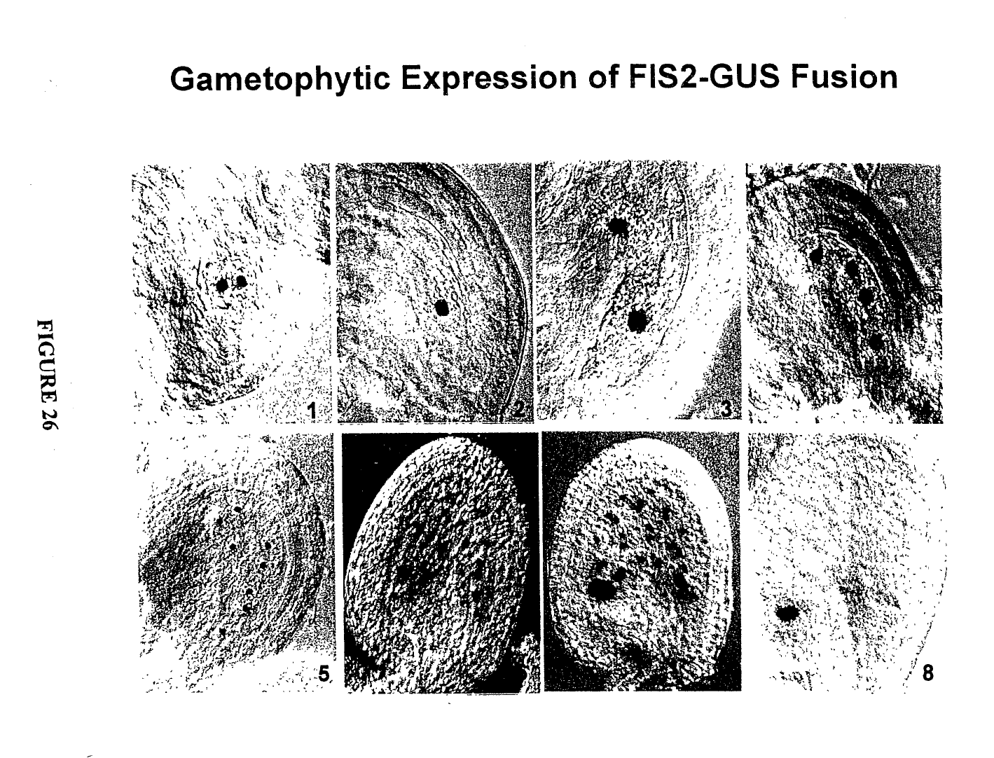

For the transformation of A. thaliana with each of the above FIS1/GUS and FIS2/GUS fusion constructs, 10 independent transformants were investigated for expression of the FIS1/GUS and FIS2/GUS fusion proteins, respectively, using standard histochemical methods. Both the FIS1/GUS and FIS2/GUS fusion proteins were found to express exclusively in the female gametophyte before and after pollination (FIGS. 25 and 26, respectively). Fusion protein expression was not detected elsewhere in the plants. Fusion protein expression was also observed in the nucleus of central cell, in the absence of fertilisation and when no nuclear division had yet occurred.

FIS2/GUS fusion protein expression (FIG. 26) was first observed particularly in the two polar nuclei in mature embryo sac initially before fusion into a central cell nucleus. Expression was then detected in the homodiploid central cell nuclei. After pollination, fusion protein expression was observed through each of the nuclear divisions that produce the endosperm, up to the stage of a 32 free endosperm nucleus. Later in development, fusion protein expression decreased, except in the endosperm nuclei at the chalazal end. Several nuclei at the chalazal end, or endosperm cysts, expressed the FIS2/GUS fusion protiens until the heart stage was reached, when the endosperm start cellularising. All expression was restricted to within the nucleus and likely to result from the putative nuclear localization domain in the FIS2 gene sequence being included in this construct. Presumably, this signal guided the FIS2/GUS fusion protein into the nucleus, as iin the case of the wild-type FIS2 protein.

The FIS1/GUS fusion showed more diffused expression than FIS2/GUS (FIG. 25), probably because this construct did not contain any nuclear localization signal. However, the pattern of FIS1/GUS fusion protein expression pattern was similar to that observed for the FIS2/GUS fusion protein. FIS1/GUS fusion protein expression was observed at the position of the central cell, however it is unclear whether FIS1/GUS expression initiated in the fused nuclei before or after nuclear fusion had occurred. After fertilization, two or four free endosperm nuclei expressing the FIS1/GUS fusion protein were detected, however expression was more diffused than for FIS2/GUS at this stage. In some cases, six free endosperm nuclei could be observed to express FIS1/GUS fusion protein, suggesting that the wild-type FIS1 protein has a similar pattern of expression to the FIS2 protein. As with the expression of the FIS2/GUS fusion protein, FIS1/GUS expression finally became localised to the chalazal end endosperm nuclei until the heart stage was reached, and declined in the other parts of endosperm.

When wild-type A. thaliana plants were pollinated using pollen derived from transgenic plants containing the expressible FIS1/GUS and FIS2/GUS fusion constructs, no FIS1/GUS or FIS2/GUS fusion protein expression detectable in the fertilized endosperm, suggesting that expression of FIS1 and FIS2 genes might occur in the maternal genome and/or that said expression may be triggered before pollination occurs.

Several putative nuclear localisation signals (NLS) were identified in the amino acid sequence of the FIS2 polypeptide (Example 11). In this regard, since both FIS2 promoter constructs directed FIS2/GUS fusion protein expression to the nucleus in the preceding Example, the FIS2 coding sequence included in these constructs must contain a functional nuclear localisation signal (NLS). However, further analysis of the FIS2 genes sequences included in these FIS2/GUS fusion constructs revealed that only the N-terminal putative NLS was present in both constructs, suggesting that this sequence is the functional NLS.

### EXAMPLE 16

### Transposon Tagging of the FIS3 Gene

The method of tagging the FIS3 gene was the same as that described in Example 5 for tagging the FIS2 gene. In the DSG tagged line designated DT51, the transposon was found to be closely linked to fis3, between the SSLP marker designated nga 162 and the RFLP marker designated ve039 (FIG. 8). The line DT51, containing Ds closely linked to fis3, was crossed with pollen from a plant containing Ac and approximately 2,000 F1 plants were screened for sectors that produced a 50:50 ratio of normal to fertilization-independent silique elongation (FIG. 10). Since the DSG element was known to be closely-linked to FIS3 in the orginal DT51 line and this element transposes to closely-linked sites on the chromosome, it is highly likely that the appearance of the fis3 mutant phenotype in these progeny lines was the result of the FIS3 gene being tagged.

The FIS3 gene is then isolated using standard procedures. First, DNA flanking the insertion site of the DSG element (FIG. 8) in the fis3-tagged mutant is cloned. A genomic DNA library is produced from the DNA of the tagged line and screened using the Ds element as a probe. Alternatively, or in addition, the gene sequences flanking the Ds element may be isolated using inverse PCR and/or tailed PCR to amplify sequences from genomic DNA or cloned genomic DNA. The nucleotide sequences of the flanking DNA may then be used to isolate the corresponding FIS3 gene sequences from a genomic library constructed using DNA derived from wild-type plants. The clones isolated from the wild-type library are subsequently used to complement the mutation in the EMS-mutagenised fis3 lines, to confirm the identity of the isolated FIS3 DNA sequences.

### EXAMPLE 17

### Isolation and Nucleotide Sequence of the Fis3 Gene

The present inventors isolated a 1372 bp full-length FIS3 cDNA from an Arabidopsis thaliana late silique cDNA library. The nucleotide sequence of this cDNA (SEQ ID NO:8) corresponded to the nucleotide sequence of the recently-described FIE gene (Ohad et al., 1999). and determined if our two alleles of fis3 (fis3-1 and 3-2) contained mutations in their FIE gene. The derived amino acid sequence of the FIS3 polypeptide is set forth herein as SEQ ID NO:3.

The cDNA clone was used to isolate a FIS3 genomic clone, by identifying the corresponding nucleotide sequence in the database of the Arabidopsis Genome Initiative (PI clone M0E17; Accession Number ABO25629). The nucleotide sequence of the FIS3 genomic clone is set forth herein as SEQ ID NO:9.

Nucleotide sequence analysis of the corresponding fis3-1 and fis3-2 mutant alleles indicated that these genes were allelic to the FIE gene. In the fis3-1 mutant allele, a G to A substitution was observed at the border of the third intron, modifying the acceptor donor site from AG to AA. In the fis3-2 mutant allele, a G to A substitution resulted in the amino acid substitution of glycine at position 104 to glutamate.

### EXAMPLE 18

### Identification of Protein-protein Interaction between FIS Proteins Using a Yeast Two Hybrid System

The FIS1, FIS2, and FIS3 cDNAs were inserted them into the yeast two-hybrid vectors pGBT9 and pGAD424, to determine whether the polypeptides encoded therefor form homodimers and/or heterodimers.

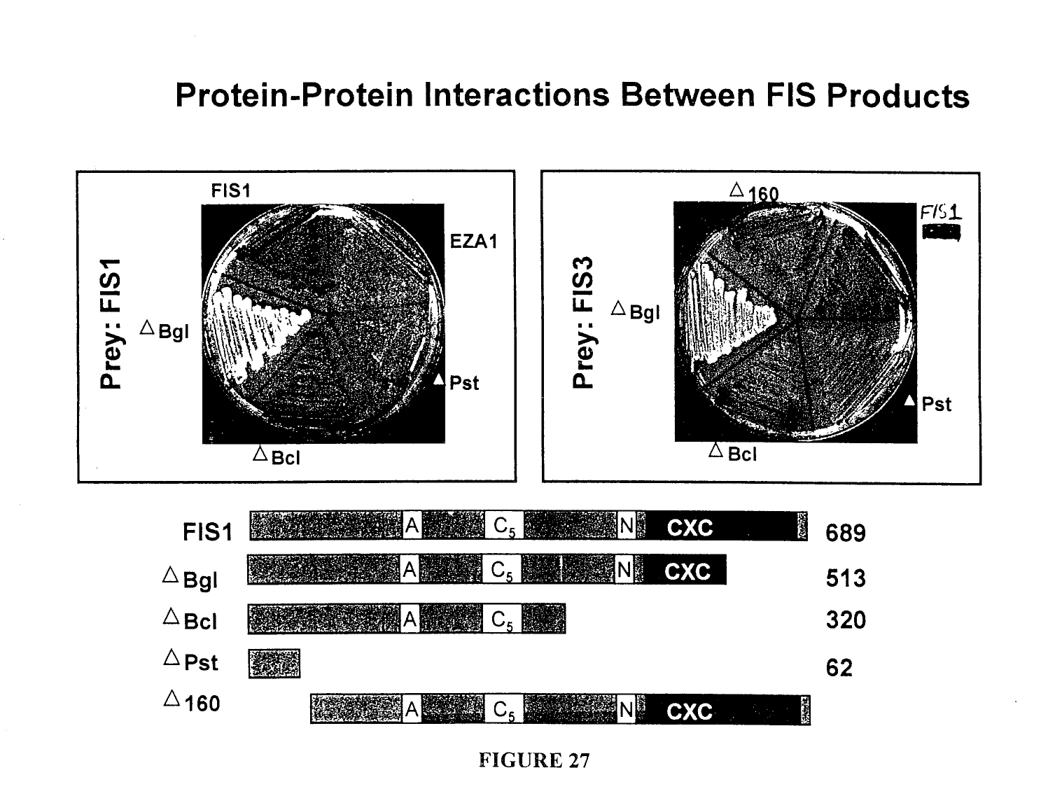

In particular, the full-length FIS1 cDNA sequence, encoding a 689 amino acid polypeptide comprising the A, C5, N, CXC and SET domains, and the deletion mutants designated: ΔBgl, encoding a 513 amino acid polypeptide and lacking the C-terminal SET domain-encoding region; ΔBcl, encoding a 320 amino acid polypeptide and lacking the C-terminal N, CXC and SET domain-encoding regions; ΔPst, encoding a 62 amino acid polypeptide and lacking the C-terminal portion of FIS1 comprising the five domain-encoding regions; and Δ160, lacking 160 bp at the 5′-end of the FIS1 cDNA, were constructed (FIG. 27). The full-length FIS2 and FIS3 cDNAs were also used. Control constructs, employing the empty vectors pGBT9 and pGAD424, or alternatively the EzA1 cDNA, were also used. Each cDNA was cloned into each vector and yeast were transformed with vectors expressing different FIS polypeptides, in the presence of adenine selection and β-Galactosidase activation, to select for cells expressing from both constructs.

Data presented in FIG. 27 to 29 indicate that the FIS1, FIS2 and FIS3 polypeptides are capable of forming certain homodimers or heterodimers.

In particular, data presented in the left panel of FIG. 27 indicates that the full-length FIS1 polypeptide is capable of forming homodimers with the full-length FIS1 polypeptide, or with truncated versions thereof comprising the A and C5 regions only (i.e. having the C-terminal 369 amino acids containing the N, CXC and SET domains deleted).

Similarly, data presented in the right panel of FIG. 27 indicates that the full-length FIS3 polypeptide is capable of forming heterodimers with the full-length FIS1 polypeptide, or alternatively, heterodimers with truncated versions of FIS1 comprising the A and C5 regions only (i.e. having the C-terminal 369 amino acids containing the N, CXC and SET domains deleted). Accordingly, the A and/or C5 regions appear to be the minimum requirement for FIS1 homodimer or FIS1/FIS3 heterodimer formation.

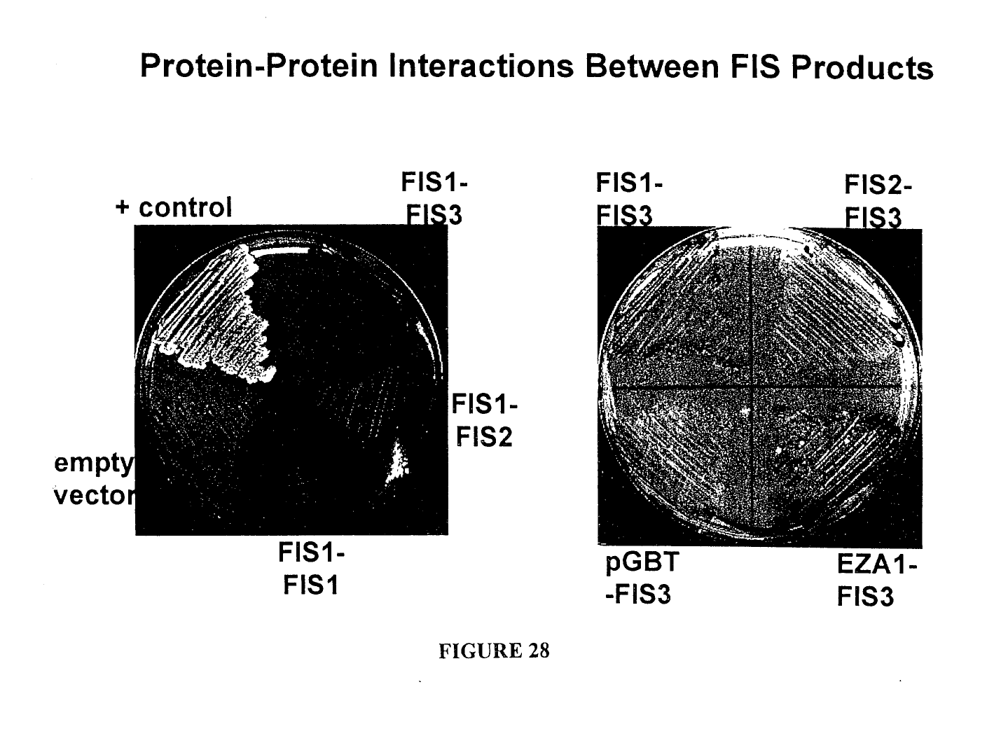

Data presented in the left panel of FIG. 28 also support the conclusion that FIS1 and FIS3 interact to an extent that is similar to FIS1/FIS, however there is only a weak interaction between FIS1 and FIS2 polypeptides in the yeast two-hybrid assay.

Data presented in the right panel of FIG. 28 indicate that EzA1 and FIS1 polypeptides both interact with the FIS3 polypeptide, however the is no significant interaction apparent in the yeast two-hybrid assay between the FIS2 and FIS3 polypeptides.

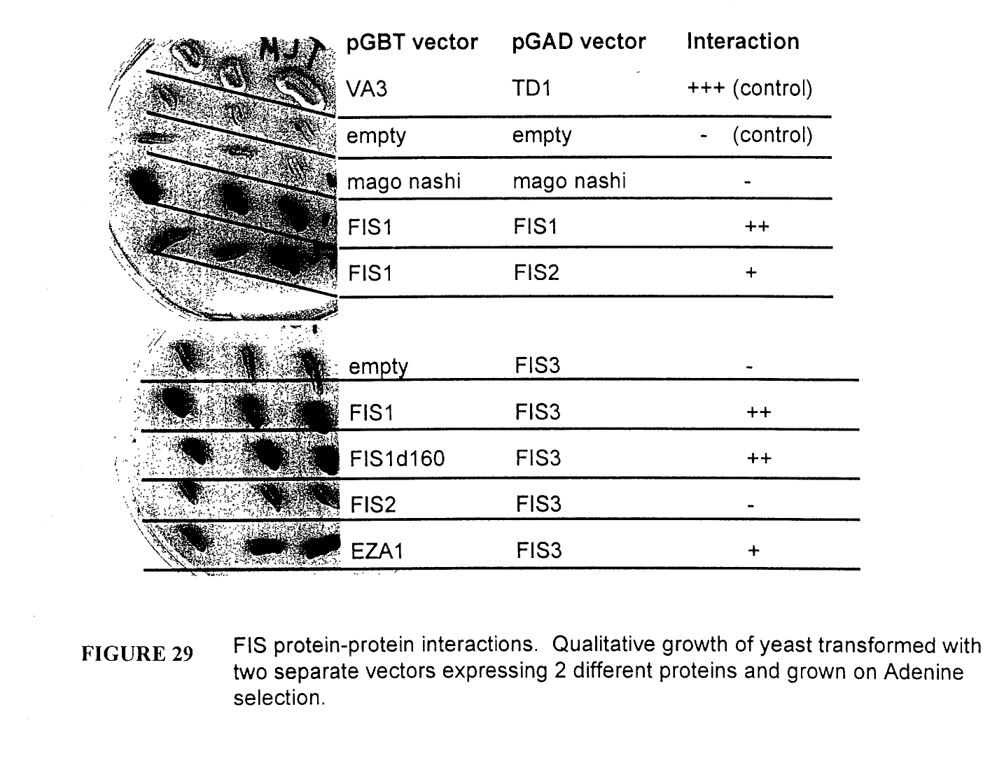

These data are also supported by the data obtained for a separate experiment, presented in FIG. 29.

The data presented herein support the hypothesis (see below) that the FIS1, FIS2 and FIS3 proteins form a complex to repress seed development in vivo.

### EXAMPLE 19

### A Screen to Isolate Genes which Regulate FIS Gene Expression

Based upon the results obtained for FIS/GUS fusion constructs described herein, genes which regulate FIS gene expression [i.e. Mother of FIS (herinafter “MOF genes”)] may encode either repressor proteins (i.e. MOF repressor genes) which inhibit expression of FIS proteins in the male gametophyte or alternatively, activator proteins (i.e. MOF activator genes) which activate or enhance expression of FIS proteins in the female gametophyte

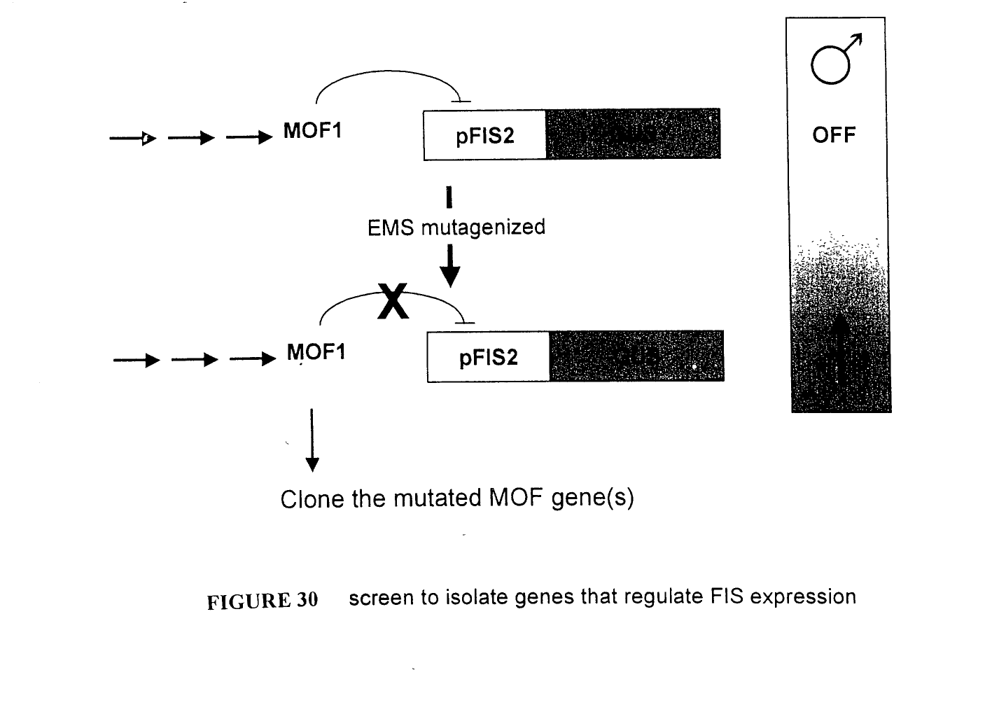

In the repressor model (FIG. 30), wild-type MOF represses FIS gene promoter function and thus, FIS gene expression is inhibited in the male gametophyte, so that FIS protein is not expressed in the pollen. Without being bound by any theory or mode of action, when a MOF gene is mutated and rendered non-functional or alternatively, encodes a non-functional MOF repressor protein, FIS protein is expressed in the male gametophyte. As a consequence, variations in the pattern of FIS protein expression in the male gametophyte will assist in identifying putative MOF gene mutants, which are useful as molecular tags to isolate the correpsonding wild-type genes using standard hybridisation and polymerase chain reaction approaches.

In the activator model, MOF proteins normally activate the expression of FIS proteins in the female gametophyte. In plants containing the FIS2/GUS reporter construct described herein, we showed that FIS-GUS was expressed in the female gamete, presumably as a consequence of the activity of MOF activator proteins.

MOF genes which regulate (i.e. enhance, activate, up-regulate, repress or down-regulate) FIS gene expression are isolated using the following procedure:

(i) seeds derived from transgenic plants containing a functional FIS2 promoter/GUS fusion construct are mutagenised;

(ii) GUS gene expression is assayed in the mutagenised lines; and

(iii) those plants having altered GUS gene expression compared to the non-mutagenized transgenic parent are selected,

wherein, if the selected plant has a mutated MOF gene or expresses an aberrant MOFgene product GUS reported gene expression is altered.

In the performance of the subject method, those plants having a mutant MOF gene, FIS protein express the GUS reporter gene in the male gametophyte. By looking at GUS staining pattern, putative MOF repressor mutants are identified and the corresponding MOF repressor genes are isolated.

The subject method can also be used to identify MOF activator genes which, when mutated, decrease GUS gene expression in the female gamete. As with the identification of MOF repressor genes described supra, putative MOF activator mutants are identified and the corresponding MOF activator genes are isolated

### EXAMPLE 20

### Discussion

Without being bound by any theory or mode of action, the FIS1, FIS2 and FIS3 polypeptides may form a complex which negatively-regulates the expression of genes that are required for the transformation of ovules into seeds or alternatively, these polypeptides may act in concert to prevent such a developmental transformation from occurring in the maternal tissues. Since seed development is linked to a diverse array of phenotypes having profound implications in agronomy, (parthenocarpy), this complex and the mode of action and regulation thereof will be pivotal to seed development.

The FIS1 and FIS2 polypeptides at least are putative transcription factors which have the potential for forming zinc-finger or zinc-binding secondary structures and, as a consequence, are likely to regulate the expression of other genes. Genes which may be regulated by FIS1-FIS2-FIS3 are likely to comprise a set of genes whose increased expression in a diverse set of organisms initiate seed development. Inappropriate activation of these genes presumably via a down regulation of FIS1-FIS2-FIS3 would initiate seed development without fertilization, producing autonomous and/or pseudogamous endosperm development.

The homology of FIS1 to polycomb group of proteins suggest that this polypeptide at least or alternatively, a FIS1-FIS2-FIS3 complex, might be involved in interacting with chromatin to maintain a status of chromatin that leads to gene inactivation. Thus, FIS1-FIS2-FIS3 may mediate epigenetic gene silencing by altering chromatin structure or methylation status.

Epigenetic gene silencing, when occurring differentially in the paternal and the maternal genome of an organism is known as imprinting and it is possible that the action of FIS1-FIS2-FIS3 is mediated via such a process. FIS1-FIS2-FIS3 may control silencing of a number of genes in the female gamete in the absence of pollination. Mutation in either of these genes would lead to an activation of the silenced genes giving rise to the fertilization independent seed phenotype. The genes controlled by the FIS1-FIS2-FIS3 complex, or a subset of such a complex, may be a subset of the imprinted genes in the female gamete that are kept silent by the combined action of these FIS polypeptides.

During normal seed development following pollination, the expression of genes derived from the paternal parent which are not silenced facilitate endosperm development in a manner similar to that which occurs in the fis mutant.

### BIBLIOGRAPHY

1. An et al. (1985) EMBO J 4:277-284;

2. Armstrong, C. L., Peterson, W. L., Buchholz, W. G., Bowen, B. A. Sulc, S. L. (1990).Plant Cell Reports 9: 335-339.

3. Asker, S. E; and Jerling, L. (1992) In: Apomixis in Plants (CRC Press, Boca Raton).

4. Ausubel, F. M., Brent, R., Kingston, R E, Moore, D. D., Seidman, J. G., Smith, J. A., and Struhl, K. (1987). In: Current Protocols in Molecular Biology. Wiley Interscience (ISBN 047150338).

5. Bendixen, C., Gangloff, S. and Rothstein R. (1994) Nucleic Acids Research. 22:1778-9.

6. Berg, P. (1993)

7. Bocher, T. W. (1951) K. Dan. Vidensk. Selsk, Biol. Skr. 6:1.

8. Bowman, J. and Koornneef, M. (1994) in Arabidopsis: An Atlas of Morphology and Development, ed. Bowman, J. (Springer, New York), pp. 351-354.

9. Chaudhury, A. M., Letham, D. S., Craig, S., and Dennis, E. S. (1993) Plant J. 4: 907-916.

10. Chaudhury, A., and Peacock, W. J. (1993)In: Abstracts of Apomixis workshop, IRRI, Manila.

11. Chaudhury, A., et al (1997)Proc. Natl. Acad. Sci. (USA) 94: 4223-4227.

12. Church, G. M.; and Gilbert, W. (1984) Proc. Natl. Acad. Sci. USA 83: 1991-1995.

13. Cole et al. (1985) In: Monoclonal antibodies in cance therapy, Alan R. Bliss Inc., pp 77-96.

14. Condorelli, G. L. et al. (1996) Cancer Research 56: 5113.

15. Christou, P., McCabe, D. E., and Swain, W. F. (1988).Plant Physiol 87: 671-674.

16. Crossway et al. (1986) Mol. Gen. Genet. 202:179-185.

17. Devereux, J., Haeberli, P., and Smithies, O. (1984) Nucl. Acids Res. 12: 387-395.

18. Dolferus, R. et al. (1994)

19. Ditta et al. (1980)

20. d'Souza, S. E., Ginsberg, M. H., and Plow, E. F. (1991) Trends Biochem. 16: 246-250.

21. Feinberg, A. P.; and Vogelstein, B. (1983) Anal. Biochem. 13: 6-13.

22. Fromm et al. (1985) Proc. Natl. Acad. Sci. (USA.) 82:5824-5828.

23. Giraudat, J., Hauge, B., Valon, C., Smalle, J., Parcy, F., and Goodman, H. M. (1992) Plant Cell 4: 1251-1261.

24. Goodrich, J., Puangsomlee, P., Martin, M., Long, D., Meyerowitz, E. M., and Coupland, G. (1997) Nature, 386: 44-51.

25. Grossniklaus, U., Vielle-Calzada, J. -P., Hoeppner, M., and Gagliano, W. (1998) Science 280: 446-450.

26. Hanahan, et al (1983)

27. Hanna, W. W., and Bashaw, E. C. (1987) Crop Sci. 27: 1136-1139.

28. Haseloff and Gerlach (1988)

29. Hauge, B. M., Hanley, C., Giraudat, J., and Goodman, H. M. (1991)In: Mapping the Arabidopsis genome (eds. Jenkins, G. I. & W. Schuch),The Company of Biologists Ltd., Cambridge.

30. Herrera-Estella et al. (1983a) Nature 303: 209-213.

31. Herrera-Estella et al. (1983b) EMBO J. 2: 987-995.

32. Herrera-Estella et al. (1985) In: Plant Genetic Engineering, Cambridge University Press, N.Y., pp 63-93.

33. Hsu, H. L. et al. (1991) Mol. Cell Biol. 11:3037.

34. Huse et al. (1989) Science 246: 1275-1281.

35. Huynh, T. V., Young, R. A., and Davis, R. W. (1985) In: DNA Cloning Vol. I: A Practical Approach (D. M. Glover, ed) IRL Press Limited, Oxford. pp49-78.

36. Iwamasa, M., Ueno, I., and Nishiura, M. (1967) Bull. Hort. Res. Sta. Jpn. Ser. 7:1-8.

37. Kohler and Milstein (1975) Nature 256: 495-499.

38. Koltunow, A. (1993) Plant Cell 5: 1425-1436.

39. Koornneef, M., Hanhart, C. J., Van Loonen-Martinet, E. P. and Van der Veen, J. H. (1987) Arabidopsis inf. Serv. 23: 46-50.

40. Kozbor et al (1983) Immunol. Today 4: 72.

41. Krens, F. A., Molendijk, L., Wullems, G. J. and Schilperoort, R. A. (1982). Nature 296: 72-74.

42. Laible, G., Wolf, A., Dorn, R., Reuter, G., Nislow, C., Lebersorger, A., Popkin, D., Pillus, L., and Jenuwein, T. (1997) EMBO J. 16: 3219-3232.

43. Langridge, J. (1957) Aust. J. Biol. Sci. 10: 243-252.

44. Lehnhardt, B., and Nitzsche, W. (1988) Angew Bot. 62: 2253.

45. Larson, R. C. et al. (1996) EMBO J. 15:1021.

46. Mahajan, M. A. et al. (1996) Oncogene 12: 2343.

47. Mansfield, S. G. (1994)In: Arabidopsis: An Atlas of Morphology and Development, cd. Bowman, J. (Springer, New York), pp. 372-377.

48. Mansfield, S. G., Briarty, L. G., and Emi, S. (1990) Can. J. Bot. 69: 447-460.

49. Mansfield, S. G., and Briarty, L. G. (1991) Can. J. Bot. 69: 461-476.

50. Mansfield, S. G., and Briarty, L. G. (1990) Arabidopsis Information Service 27: 53-64.

51. McPherson, M. J., Quirke, P., and Taylor, G. R. (1991)In: PCR: A Practical Approach. (series editors, D. Rickwood and B. D. Hames) IRL Press Limited, Oxford. pp1-253.

52. Meisner and Michael (1997)

53. Needleman and Wunsch (1970) J. Mol. Biol. 48:443-453.

54. Osada, et al. (1995) Proc. Natl. Acad. Sci. (USA.) 92: 9585.

55. Ozias-Akins, P., Lubbers, E. L., Hanna, W. W., and McNay, J. W. (1993) Theoretical and Applied Genetics 85: 632-638.

56. Parlevliet, J. E., and Cameron, J. W. (1959) Proc. Am. Soc. Hort. Sci. 74: 252-260.

57. Paszkowski et al. (1984) EMBO J. 3:2717-2722.

58. Peacock, W. J. (1992) Apomixis Newsletter 4: 3-7.

59. Peacock, W. J. (1995)

60. Poutney, D. L., Tiwari, R., and Egan, J. B. (1997 Protein Science 6: 892-902.

61. Robinson-Beers, K., Pruitt, R. E., and Gasser, C. S. (1992) Plant Cell 4: 1237-1249.

62. Roy, B. A., and Riseberg, L. H. (1989) J. Heredity 80: 506-508.

63. Ruoslahti, E., and Piersbacher, M. D. (1986) Cell 44: 517-518.

64. Sakai et al. (1995)

65. Sambrook, J., Fritsch, E. F., and Maniatis, T. (1989)In: Molecular Cloning, a Laboratory Manual 2nd Edition, Cold Spring Harbor N.Y.: Cold Spring Harbor Laboratory Press.

66. Sanford, J. C., Klein, T. M., Wolf, E. D., and Allen, N. (1987). Particulate Science and Technology 5: 27-37.

67. Staden (1982) Nucl. Acids. Res. 10: 2951-2961.

68. Stanojevic et al. (1989)

69. Sun and Kamiya (1994)

70. Tague and Goodman (1995)

71. Takatsuji et al. (1991)

72. Takatsuji et al. (1994)

73. Taylor (1982)

74. Thompson, J. D., Higgins, D. G., and Gibson, T. J. (1994) Nucl. Acids Res. 22: 4673-4680.

75. Treisman and Desplan (1989)

76. Valvekens (1988)

77. Vidal, M. et al. (1996a) Proc. Natl. Acad. Sci. (USA.) 93: 10315.

78. Vidal, M. et al. (1996b) Proc. Natl. Acad. Sci. (USA.) 93: 10321.

79. Yang, M. et al. (1995) Nucleic Acid Sequence 23: 1152.

80. Zhang, J. et al.(1996) Anal Biochem.242: 68.

81. Marck, et al. (1988)

82. Dellaporta et al. (1983)

83. Roder et al. (1986)

84. Ohad et al (1999)

85. Ozias-Akins (1998)

We claim:

1. A method of inducing the development of seed in a plant, comprising introducing into said plant or a parent of said plant, genetic material which reduces expression of a gene in one or more female reproductive cells of said plant, wherein said gene hybridizes to SEQ ID NO:6 or a complementary form thereof under stringent conditions, wherein the reduction of expression of the gene is sufficient to induce development of seed in the plant.

2. The method of claim 1, wherein the gene encodes a polypeptide comprising the amino acid sequence motif C-X₂-C-Xₙ-H-X₄-H, wherein n=10 to 15 amino acid residues in length and wherein numerical values indicate the number of consecutive multiple occurrences of a particular amino acid residue.

3. The method of claim 1, wherein the gene encodes a polypeptide which comprises SEQ ID NO:2.

4. The method of claim 1, wherein the expression of the gene is reduced by a method comprising mutagenesis of the gene.

5. The method of claim 4 wherein the mutagenesis produces a null allele of the gene.

6. The method of claim 4 wherein the mutagenesis is performed using a chemical mutagen.

7. The method of claim 6 wherein the chemical mutagen is EMS.

8. The method of claim 4, wherein the mutagenesis comprises insertion of a nucleic acid molecule into the gene.

9. The method of claim 8, wherein the nucleic acid molecule comprises a member selected from the group consisting of T-DNA, a gene targeting molecule and a transposon.

10. The method of claim 1, wherein the seed comprises an endosperm.

11. The method of claim 1, wherein the seed lacks a functional embryo structure.

12. The method of claim 11, wherein the seed is a soft seed.

13. The method of claim 1, wherein the seed is able to germinate.

14. The method of claim 1, wherein the seed is autonomously produced.

15. The method of claim 14, wherein the seed is produced independent of fertilization.

16. A method of producing seedless or soft-seeded fruit in a plant, comprising introducing into said plant or a parent of said plant genetic material which reduces expression of a gene in one or more female reproductive cells of said plant, wherein said gene hybridizes to SEQ ID NO:6 or a complementary form thereof under stringent conditions and wherein the reduction of expression of the gene is sufficient to produce seedless or soft-seeded fruit in the plant.

17. The method of claim 1, wherein the gene comprises the nucleotide sequence set forth in SEQ ID NO:6 or SEQ ID NO:7.

18. The method of claim 16, wherein the gene comprises the nucleotide sequence set forth in SEQ ID NO:6 or SEQ ID NO:7.

19. The method of claim 1, wherein the plant is a transgenic plant.

20. The method of claim 1, wherein the genetic material encodes an antisense, a ribozyme, sense or gene silencing RNA.

21. The method of claim 16, wherein the genetic material encodes an antisense, a ribozyme, sense or gene silencing RNA.

22. The method of claim 16, wherein the expression of said gene is reduced by a method comprising mutagenesis of the gene.

23. The method of claim 22, wherein the mutagenesis comprises the insertion of a nucleic acid molecule into the gene.

24. The method of claim 23, wherein the nucleic acid molecule comprises a member selected from the group consisting of T-DNA, a gene targeting molecule and a transposon.

25. A plant generated by the process of introducing genetic material to a cell of a plant, which genetic material reduces expression of a gene which hybridizes to SEQ ID NO:6 or a complementary form thereof under stringent conditions and then regenerating a plant from said plant cell.

26. The plant of claim 25, comprising a nucleic acid molecule inserted into said gene.

27. A progeny or seeds of the plant of claim 25, wherein the progeny or seeds comprise the genetic material.

28. A progeny or seeds of the plant of claim 26, wherein the progeny or seeds comprise the nucleic acid molecule inserted into said gene.

29. A plant comprising seed developed by the method of claim 1.

30. A progeny plant or seeds obtained from the plant of claim 29, wherein said progeny plant or seeds comprise the genetic material.

31. A seedless or soft-seeded fruit produced by the method of claim 23, wherein the seedless or soft-seeded fruit comprises the nucleic acid molecule inserted into the gene.

32. A seedless or soft-seeded fruit produced by the method of claim 16, wherein the seedless or soft-seeded fruit comprises the genetic material.

33. The method of claim 20, wherein the genetic material encodes an antisense RNA.

34. The method of claim 20, wherein the genetic material encodes a ribozyme.

35. The method of claim 20, wherein the genetic material encodes sense RNA.

36. The method of claim 20, wherein the genetic material encodes gene silencing RNA.

37. The plant of claim 25 wherein the genetic material encodes an antisense, a ribozyme, sense or gene silencing RNA.

38. The plant of claim 37, wherein the genetic material encodes an antisense RNA.

39. The plant of claim 37, wherein the genetic material encodes a ribozyme.

40. The plant of claim 37, wherein the genetic material encodes sense RNA.

41. The plant of claim 37, wherein the genetic material encodes gene silencing RNA.
